SEQUENCE LISTING (38 sequences)

SEQ ID NO: 1 - DNA, 30 bp, Artificial Sequence
  Description of Artificial Sequence Synthetic primer
tttttttttttttttttttttttttttttt

SEQ ID NO: 2 - DNA, 14 bp, Artificial Sequence
  Description of Artificial Sequence Synthetic primer
cacgagccgccagc

SEQ ID NO: 3 - DNA, 23 bp, Artificial Sequence
  Description of Artificial Sequence Synthetic primer
tctgcaacagatgacgcaataag

SEQ ID NO: 4 - DNA, 9 bp, Artificial Sequence
  Description of Artificial Sequence Synthetic probe
cgcctgccg

SEQ ID NO: 5 - DNA, 10 bp, Artificial Sequence
  Description of Artificial Sequence Synthetic probe
gcagaatgtc

SEQ ID NO: 6 - DNA, 20 bp, Artificial Sequence
  Description of Artificial Sequence Synthetic primer
gaagcagccatggtctaagc

SEQ ID NO: 7 - DNA, 24 bp, Artificial Sequence
  Description of Artificial Sequence Synthetic primer
ccttgtcgactgaagagcaatatg

SEQ ID NO: 8 - DNA, 9 bp, Artificial Sequence
  Description of Artificial Sequence Synthetic probe
cggcccagc

SEQ ID NO: 9 - DNA, 13 bp, Artificial Sequence
  Description of Artificial Sequence Synthetic probe
cacgatgatacat

SEQ ID NO: 10 - DNA, 17 bp, Artificial Sequence
  Description of Artificial Sequence Synthetic primer
ccggaagtgggtctcgt

SEQ ID NO: 11 - DNA, 23 bp, Artificial Sequence
  Description of Artificial Sequence Synthetic primer
ttcttcatagccttcccgatacc

SEQ ID NO: 12 - DNA, 9 bp, Artificial Sequence
  Description of Artificial Sequence Synthetic probe
tcgtgatgg

SEQ ID NO: 13 - DNA, 16 bp, Artificial Sequence
  Description of Artificial Sequence Synthetic probe
cggatgagaggtttca

SEQ ID NO: 14 - DNA, 15 bp, Artificial Sequence
  Description of Artificial Sequence Synthetic primer
atggcagcggacgga

SEQ ID NO: 15 - DNA, 18 bp, Artificial Sequence
  Description of Artificial Sequence Synthetic primer
ggctcaggtaggcaagga

SEQ ID NO: 16 - DNA, 9 bp, Artificial Sequence
  Description of Artificial Sequence Synthetic probe
ccacgtcga

SEQ ID NO: 17 - DNA, 18 bp, Artificial Sequence
  Description of Artificial Sequence Synthetic probe
atatcctgcaggtgatct

SEQ ID NO: 18 - DNA, 18 bp, Artificial Sequence
  Description of Artificial Sequence Synthetic primer
tgaaagctgccgctgaag

SEQ ID NO: 19 - DNA, 15 bp, Artificial Sequence
  Description of Artificial Sequence Synthetic primer
ccctgggctcgccat

SEQ ID NO: 20 - DNA, 9 bp, Artificial Sequence
  Description of Artificial Sequence Synthetic probe
cggacccgc

SEQ ID NO: 21 - DNA, 10 bp, Artificial Sequence
  Description of Artificial Sequence Synthetic probe
gatggagacc

SEQ ID NO: 22 - DNA, 19 bp, Artificial Sequence
  Description of Artificial Sequence Synthetic primer
gtgacaggctacgcaacag

SEQ ID NO: 23 - DNA, 22 bp, Artificial Sequence
  Description of Artificial Sequence Synthetic primer
tcctgagttttccttgctagct

SEQ ID NO: 24 - DNA, 9 bp, Artificial Sequence
  Description of Artificial Sequence Synthetic probe
agggcgcca

SEQ ID NO: 25 - DNA, 9 bp, Artificial Sequence
  Description of Artificial Sequence Synthetic probe
gatggctga

SEQ ID NO: 26 - DNA, 16 bp, Artificial Sequence
  Description of Artificial Sequence Synthetic primer
gtcgaccccgcttcca

SEQ ID NO: 27 - DNA, 20 bp, Artificial Sequence
  Description of Artificial Sequence Synthetic primer
ggtcatgccagtagccaatg

SEQ ID NO: 28 - DNA, 9 bp, Artificial Sequence
  Description of Artificial Sequence Synthetic probe
atgcttccc

SEQ ID NO: 29 - DNA, 16 bp, Artificial Sequence
  Description of Artificial Sequence Synthetic probe
tgtgcaagaggtttgc

SEQ ID NO: 30 - DNA, 14 bp, Artificial Sequence
  Description of Artificial Sequence Synthetic primer
gcagcgggcagacg

SEQ ID NO: 31 - DNA, 18 bp, Artificial Sequence
  Description of Artificial Sequence Synthetic primer
atggcggcagatcagtct

SEQ ID NO: 32 - DNA, 9 bp, Artificial Sequence
  Description of Artificial Sequence Synthetic probe
catcgctct

SEQ ID NO: 33 - DNA, 16 bp, Artificial Sequence
  Description of Artificial Sequence Synthetic probe
gagaacagacctggtg

SEQ ID NO: 34 - DNA, 16 bp, Artificial Sequence
  Description of Artificial Sequence Synthetic oligonucleotide
aaaaaaaaaaaaaaaa

SEQ ID NO: 35 - DNA, 11 bp, Artificial Sequence
  Description of Artificial Sequence Synthetic oligonucleotide
aaaaaaaaaaa

SEQ ID NO: 36 - DNA, 11 bp, Artificial Sequence
  Description of Artificial Sequence Synthetic oligonucleotide
ttttttttttt

SEQ ID NO: 37 - DNA, 28 bp, Artificial Sequence
  Description of Artificial Sequence Synthetic oligonucleotide
cattatcatggaaaggcctgcgttcaat

SEQ ID NO: 38 - DNA, 52 bp, Artificial Sequence
  Description of Artificial Sequence Synthetic oligonucleotide
aggtccctctgtgctcgggtccaagctgtcaggcttcctccccagctcatgg

(19) United States
(12) Patent Application Publication
(10) Pub. No.: US 2021/0277471 A1
(43) Pub. Date: Sep. 9, 2021

(54) CELL POPULATION ANALYSIS USING SINGLE NUCLEOTIDE POLYMORPHISMS FROM SINGLE CELL TRANSCRIPTOMES

(71) Applicants: 10X Genomics, Inc., Pleasanton, CA (US); Fred Hutchinson Cancer Research Center, Seattle, WA (US)

(72) Inventors: Xinying Zheng, San Jose, CA (US); Jason H. Bielas, Seattle, WA (US); Mark T. Gregory, Seattle, WA (US); Benjamin Hindson, Pleasanton, CA (US); Tarjei Sigurd Mikkelsen, Dublin, CA (US); Ryan Wilson, Redwood City, CA (US); Paul Ryvkin, San Jose, CA (US)

(21) Appl. No.: 17/029,756

(22) Filed: Sep. 23, 2020

Related U.S. Application Data
(63) Continuation of application No. 15/430,298, filed on Feb. 10, 2017, now abandoned.
(60) Provisional application No. 62/293,966, filed on Feb. 11, 2016.
(60) Provisional application No. 62/365,961, filed on Jul. 22, 2016.
(60) Provisional application No. 62/365,962, filed on Jul. 22, 2016.

(51) Int. Cl. C12Q 1/6881; C12N 15/10

(52) U.S. Cl. CPC C12Q 1/6881; G01N 2015/1006; C12N 15/1065; C12N 15/1093

(57) ABSTRACT

The disclosure provides methods and systems for producing single cell RNA sequencing data. Single nucleotide polymorphisms (SNPs) identified in such data can be used to distinguish subpopulations of cells within a mixed population.

### CROSS-REFERENCE TO RELATED APPLICATIONS

This application is a continuation of U.S. application Ser. No. 15/430,298, filed on Feb. 10, 2017, which claims priority to U.S. Provisional Patent Application No. 62/293,966 filed Feb. 11, 2016, U.S. Provisional Patent Application No. 62/365,961 filed Jul. 22, 2016, and U.S. Provisional Patent Application No. 62/365,962 filed Jul. 22, 2016 each of which applications is herein incorporated by reference in its entirety for all purposes.

The instant application contains a Sequence Listing which has been submitted electronically in ASCII format and is hereby incorporated by reference in its entirety. Said ASCII copy, created on Apr. 18, 2017, is named 43487-747_201_SL.txt and is 9,064 bytes in size.

### BACKGROUND

Significant advances in analyzing and characterizing biological and biochemical materials and systems, including but not limited to the characterization of transcriptomes of individuals cells, have led to unprecedented advances in understanding complex biological systems. Among these advances, technologies that target and characterize cells at a single-cell level have yielded some of the most groundbreaking results, including advances in the use and exploitation of genetic amplification technologies and nucleic acid sequencing technologies.

Knowledge of individual components of biological systems can be useful for understanding the systems themselves. Various cellular analysis techniques can be used to investigate these components. Cellular analysis techniques include ensemble measurements where averages are taken over a population. Ensemble measurements can be useful for homogeneous populations. For heterogeneous cell populations, however, cellular analysis of populations can result in misleading averages. For example, in the study of the transcriptome, or the set of messenger RNA molecules of a cell, ensemble measurements can overlook small changes in cells and/or the presence of a minor cell population or minor cell populations with properties different from the majority. Analysis of cell populations at a single-cell level, therefore, can be useful to observe and/or evaluate cellular heterogeneity at a single-cell level.

Single cell RNA-sequencing (scRNA-seq), for example, can be used to dissect transcriptomic heterogeneity that can often be masked in population-averaged measurements. Existing scRNA-seq methods face practical challenges when scaling to tens of thousands of cells (or greater) or when it may be necessary to capture as many cells as possible from a limited sample. Commercially-available, microfluidic-based approaches may be limited, for example, by low throughput. Plate-based methods can often require time-consuming fluorescence-activated cell sorting into many plates that are processed separately. Droplet-based techniques have enabled processing of tens of thousands of cells in a single experiment, but may require generation of custom microfluidic devices and reagents.

### SUMMARY

In view of the foregoing, the present disclosure provides methods, systems and compositions for single-cell analysis, including single-cell transcriptome analysis. In an aspect, the present disclosure provides a fully-integrated, droplet-based system that enables 3′ mRNA digital counting of up to tens of thousands of single cells. In some embodiments, approximately 50% of cells loaded into the system can be captured, and up to 8 samples can be processed in parallel. Reverse transcription (RT) can occur inside each droplet, and barcoded cDNAs can be amplified in bulk. In some embodiments, the resulting libraries undergo next-generation sequencing, for example, Illumina short-read sequencing. An analysis pipeline can then process the sequencing data and enable automated cell clustering analysis.

In an aspect, the present disclosure provides a method of distinguishing a minor cell population from a major cell population in a heterogeneous cell sample. The method comprises (a) partitioning a plurality of cells of a heterogeneous cell sample into a plurality of droplets, wherein upon partitioning, a given droplet of the plurality of droplets comprises a given cell of the plurality of cells and a given bead of a plurality of beads comprising a plurality of oligonucleotide barcodes, wherein the given cell comprises a first set of polynucleotides; (b) subjecting the first set of polynucleotides to nucleic acid amplification under conditions sufficient to generate a second set of polynucleotides, wherein a given polynucleotide of the second set of polynucleotides comprises (i) a segment having a sequence of a polynucleotide of the first set or a complement thereof and (ii) a segment having a sequence of a oligonucleotide barcode of the plurality of oligonucleotide barcodes or a complement thereof; (c) generating a library of polynucleotides from a pool of polynucleotides comprising a plurality of second sets of polynucleotides, including the second set of polynucleotides, from the plurality of droplets; (d) subjecting the library of polynucleotides to sequencing to yield sequencing reads, wherein barcode sequences of the plurality of oligonucleotide barcodes associate sequencing reads with individual cells of the plurality of cells of the heterogeneous cell sample; and (e) processing the sequencing reads associated with individual cells of the plurality of cells of the heterogeneous cell sample to generate (i) a first set of genetic aberrations corresponding to the minor cell population and (ii) a second set of genetic aberrations corresponding to the major cell population, which first and second set of genetic aberrations differentiate a cell of the minor cell population from a cell of the major cell population. In some embodiments, the method further comprises, subsequent to (a), releasing the first set of polynucleotides from the given cell into the given droplet.

In some embodiments, the given bead of the given droplet is a gel bead. In some embodiments, the given bead of the given droplet comprises at least 1,000,000 oligonucleotide barcodes. In some embodiments, each oligonucleotide barcode of the given bead of the given droplet comprises a barcode sequence identical to all other oligonucleotide barcodes of the given bead of the given droplet and a molecular identifier sequence not identical to all other oligonucleotide barcodes of the given bead of the given droplet. In some embodiments disclosed herein, the method further comprises applying a stimulus to the given droplet to release the oligonucleotide barcodes from the given bead into the given droplet.

In some embodiments, the first set of genetic aberrations and the second set of genetic aberrations comprise single nucleotide variants (SNVs). In some embodiments, each of the first and second set of genetic aberrations comprises at least 30 SNVs. In some embodiments, each of the first and second set of genetic aberrations comprises at least 40 SNVs. In some embodiments, each of the first and second set of genetic aberrations comprises at least 50 SNVs. In some embodiments, each of the first and second set of genetic aberrations comprises at least 100 SNVs. In any of the aforementioned embodiments, the first set of genetic aberrations and the second set of genetic aberrations do not intersect (do not share members).

In some embodiments, the major cell population comprises at least two cell types. In some embodiments, the minor cell population represents less than 50% of the heterogeneous cell sample. In some embodiments, the minor cell population represents greater than or equal to about 1% of the heterogeneous cell sample.

In some embodiments, the method further comprises determining a percentage of the heterogeneous cell sample represented by the major cell population. In some embodiments, the major cell population represents greater than about 50% of the heterogeneous cell sample. In some embodiments, the major cell population represents less than 100% of the heterogeneous cell sample.

In some embodiments, the method further comprises determining a percentage of the heterogeneous cell sample represented by the minor cell population. In some embodiments, the minor cell population represents less than about 50% of the heterogeneous cell sample. In some embodiments, the minor cell population represents at least 1% of the heterogeneous cell sample. In some embodiments, the minor cell population represents at least 2% of the heterogeneous cell sample. In some embodiments, the minor cell population represents at least 3% of the heterogeneous cell sample. In some embodiments, the minor cell population represents at least 4% of the heterogeneous cell sample. In some embodiments, the minor cell population represents at least 5% of the heterogeneous cell sample. In any of the aforementioned embodiments, the percentage of the heterogeneous cell sample represented by the minor cell population is determined at a sensitivity of at least about 95%. In any of the aforementioned embodiments, the percentage is determined at a sensitivity of at least about 97%. In any of the aforementioned embodiments, the percentage is determined at a sensitivity of at least about 98%.

In some embodiments disclosed herein, nucleic acid amplification reagents are co-partitioned in the given droplet. In some embodiments, the nucleic acid amplification reagents comprise a polymerase. In some embodiments, the nucleic acid amplification reagents comprise a template switching oligonucleotide.

In some embodiments, the heterogeneous cell sample comprises cells obtained from a biological sample. In some embodiments, the biological sample comprises bone marrow. In some embodiments, the biological sample comprising bone marrow is obtained from a subject undergoing or having undergone a bone marrow transplant. In any of the aforementioned embodiments, the heterogeneous cell sample comprises cells that have been cryopreserved.

In an aspect, the present disclosure provides a method of distinguishing a first cell population from a second cell population in a heterogeneous cell sample. The method comprises (a) partitioning a plurality of cells of a heterogeneous cell sample into a plurality of droplets, wherein upon partitioning, a given droplet of the plurality of droplets comprises a given cell of the plurality of cells and a given bead of a plurality of beads comprising a plurality of oligonucleotide barcodes, wherein the given cell comprises a first set of polynucleotides; (b) subjecting the first set of polynucleotides to nucleic acid amplification under conditions sufficient to generate a second set of polynucleotides, wherein a given polynucleotide of the second set of polynucleotides comprises (i) a segment having a sequence of a polynucleotide of the first set or a complement thereof and (ii) a segment having a sequence of a oligonucleotide barcode of the plurality of oligonucleotide barcodes or a complement thereof; (c) generating a library of polynucleotides from a pool of polynucleotides comprising a plurality of second sets of polynucleotides, including the second set of polynucleotides, from the plurality of droplets; (d) subjecting the library of polynucleotides to sequencing to yield sequencing reads, wherein barcode sequences of the plurality of oligonucleotide barcodes associate sequencing reads with individual cells of the plurality of cells of the heterogeneous cell sample; and (e) determining a percentage of the heterogeneous cell sample represented by the first cell population using a first set of genetic aberrations corresponding to the first cell population and a second set of genetic aberrations corresponding to the second cell population obtained from processing the sequencing reads associated with individual cells of the heterogeneous cell sample. In some embodiments, the method further comprises, subsequent to (a), releasing the first set of polynucleotides from the given cell into the given droplet.

In some embodiments, the given bead of the given droplet is a gel bead. In some embodiments, the given bead of the given droplet comprises at least 1,000,000 oligonucleotide barcodes. In some embodiments, each oligonucleotide barcode of the given bead of the given droplet comprises a barcode sequence identical to all other oligonucleotide barcodes of the given bead of the given droplet and a molecular identifier sequence not identical to all other oligonucleotide barcodes of the given bead of the given droplet. In some embodiments disclosed herein, the method further comprises applying a stimulus to the given droplet to release the oligonucleotide barcodes from the given bead into the given droplet.

In some embodiments, the first set of genetic aberrations and the second set of genetic aberrations comprise single nucleotide variants (SNVs). In some embodiments, each of the first and second set of genetic aberrations comprises at least 30 SNVs. In some embodiments, each of the first and second set of genetic aberrations comprises at least 40 SNVs. In some embodiments, each of the first and second set of genetic aberrations comprises at least 50 SNVs. In some embodiments, each of the first and second set of genetic aberrations comprises at least 100 SNVs. In some embodiments disclosed herein, the first set of genetic aberrations and the second set of genetic aberrations do not intersect (do not share members).

In some embodiments, the second cell population comprises at least two cell types. In some embodiments, the first cell population represents less than 50% of the heterogeneous cell sample. In some embodiments, the first cell population represents greater than or equal to about 1% of the heterogeneous cell sample.

In some embodiments disclosed herein, the method further comprises determining a percentage of the heterogeneous cell sample represented by the second cell population. In some embodiments, the second cell population represents greater than about 50% of the heterogeneous cell sample. In some embodiments, the second cell population represents less than 100% of the heterogeneous cell sample.

In some embodiments, the first cell population represents at least 1% of the heterogeneous cell sample. In some embodiments, first cell population represents at least 2% of the heterogeneous cell sample. In some embodiments, the first cell population represents at least 3% of the heterogeneous cell sample. In some embodiments, first cell population represents at least 4% of the heterogeneous cell sample. In some embodiments, the first cell population represents at least 5% of the heterogeneous cell sample. In any of the aforementioned embodiments, the percentage is determined at a sensitivity of at least about 95%. In any of the aforementioned embodiments, the percentage is determined at a sensitivity of at least about 97%. In any of the aforementioned embodiments, percentage is determined at a sensitivity of at least about 98%.

In some embodiments disclosed herein, nucleic acid amplification reagents are co-partitioned in the given droplet. In some embodiments, the nucleic acid amplification reagents comprise a polymerase. In some embodiments, the nucleic acid amplification reagents comprise a template switching oligonucleotide.

In some embodiments, the heterogeneous cell sample comprises cells obtained from a biological sample. In some embodiments, the biological sample comprises bone marrow. In some embodiments, the biological sample comprising bone marrow is obtained from a subject undergoing or having undergone a bone marrow transplant. In any of the aforementioned embodiments, the heterogeneous cell sample comprises cells that have been cryopreserved.

In an aspect, the present disclosure provides a method of determining a percentage of a cell population in a heterogeneous cell sample at a sensitivity of at least about 95%, wherein the cell population represents less than about 10% of the heterogeneous cell sample. The method comprises (a) partitioning a plurality of cells of a heterogeneous cell sample into a plurality of droplets, wherein upon partitioning, a given droplet of the plurality of droplets comprises a given cell of the plurality of cells and a given bead of a plurality of beads comprising a plurality of oligonucleotide barcodes, wherein the given cell comprises a first set of polynucleotides; (b) subjecting the first set of polynucleotides to nucleic acid amplification under conditions sufficient to generate a second set of polynucleotides, wherein a given polynucleotide of the second set of polynucleotides comprises (i) a segment having a sequence of a polynucleotide of the first set or a complement thereof and (ii) a segment having a sequence of a oligonucleotide barcode or a complement thereof; (c) generating a library of polynucleotides from a pool of polynucleotides comprising a plurality of second sets of polynucleotides, including the second set of polynucleotides, from the plurality of droplets; (d) subjecting the library of polynucleotides to sequencing to yield sequencing reads, wherein barcode sequences of the plurality oligonucleotide barcodes associate sequencing reads with individual cells of the plurality of cells of the heterogeneous cell sample; (e) determining, with a sensitivity of at least about 95%, a percentage of the heterogeneous cell sample represented by the cell population using a first set of genetic aberrations and a second set of genetic aberrations obtained from processing the sequencing reads associated with individual cells of the heterogeneous cell sample, wherein the cell population represents less than about 10% of the heterogeneous cell sample. In some embodiments, the method further comprises, subsequent to (a), releasing the first set of polynucleotides from the given cell into the given droplet.

In some embodiments, the given bead of the given droplet is a gel bead. In some embodiments, the given bead of the given droplet comprises at least 1,000,000 oligonucleotide barcodes. In some embodiments, each oligonucleotide barcode of the given bead of the given droplet comprises a barcode sequence identical to all other oligonucleotide barcodes of the given bead of the given droplet and a molecular identifier sequence not identical to all other oligonucleotide barcodes of the given bead of the given droplet. In some embodiments disclosed herein, the method further comprises applying a stimulus to the given droplet to release the oligonucleotide barcodes from the given bead into the given droplet.

In some embodiments, the first set of genetic aberrations and the second set of genetic aberrations comprise single nucleotide variants (SNVs). In some embodiments, each of the first and second set of genetic aberrations comprises at least 30 SNVs. In some embodiments, each of the first and second set of genetic aberrations comprises at least 40 SNVs. In some embodiments, each of the first and second set of genetic aberrations comprises at least 50 SNVs. In some embodiments, each of the first and second set of genetic aberrations comprises at least 100 SNVs. In some embodiments disclosed herein, the first set of genetic aberrations and the second set of genetic aberrations do not intersect (do not share members).

In some embodiments, the heterogeneous cell sample comprises at least two cell types. In some embodiments, the heterogeneous cell sample comprises at least three cell types. In some embodiments, the cell population represents greater than or equal to about 1% of the heterogeneous cell sample. In some embodiments, the cell population represents at least 1% of the heterogeneous cell sample. In some embodiments, the cell population represents at least 2% of the heterogeneous cell sample. In some embodiments, the cell population represents at least 3% of the heterogeneous cell sample. In some embodiments, the cell population represents at least 4% of the heterogeneous cell sample. In some embodiments, the cell population represents at least 5% of the heterogeneous cell sample. In any of the aforementioned embodiments, the percentage of the heterogeneous cell sample represented by the cell population is determined at a sensitivity of at least about 96%. In any of the aforementioned embodiments, the percentage is determined at a sensitivity of at least about 97%. In any of the aforementioned embodiments, the percentage is determined at a sensitivity of at least about 98%. In any of the aforementioned embodiments, the percentage is determined at a sensitivity of at least about 99%.

In some embodiments disclosed herein, nucleic acid amplification reagents are co-partitioned in the given droplet. In some embodiments, the nucleic acid amplification reagents comprise a polymerase. In some embodiments, the nucleic acid amplification reagents comprise a template switching oligonucleotide.

In some embodiments, the heterogeneous cell sample comprises cells obtained from a biological sample. In some embodiments, the biological sample comprises bone marrow. In some embodiments, the biological sample comprising bone marrow is obtained from a subject undergoing or having undergone a bone marrow transplant.

In any of the aforementioned embodiments, the heterogeneous cell sample comprises cells that have been cryopreserved.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates a microfluidic channel structure for partitioning individual or small groups of cells.

FIG. 2 schematically illustrates a microfluidic channel structure for co-partitioning cells and beads or microcapsules comprising additional reagents.

FIGS. 3A-3F schematically illustrates an example process for amplification and barcoding of cell's nucleic acids.

FIG. 4 provides a schematic illustration of use of barcoding of cell's nucleic acids in attributing sequence data to individual cells or groups of cells for use in their characterization.

FIG. 5 provides a schematic illustrating cells associated with labeled cell-binding ligands.

FIG. 6 provides a schematic illustration of an example workflow for performing RNA analysis using the methods described herein.

FIG. 7 provides a schematic illustration of an example barcoded oligonucleotide structure for use in analysis of ribonucleic (RNA) using the methods described herein.

FIG. 8 provides an image of individual cells co-partitioned along with individual barcode bearing beads

FIGS. 9A-E provides schematic illustration of example barcoded oligonucleotide structures for use in analysis of RNA and example operations for performing RNA analysis. FIGS. 9A-E disclose SEQ ID NO:34

FIG. 10 provides schematic illustration of example barcoded oligonucleotide structure for use in example analysis of RNA and use of a sequence for in vitro transcription. FIG. 10 discloses SEQ ID NO: 34

FIG. 11 provides schematic illustration of an example barcoded oligonucleotide structure for use in analysis of RNA and example operations for performing RNA analysis. FIG. 10 discloses SEQ ID NOS 35, 36, 35 and 36 respectively, in order of appearance

FIGS. 12A-B provides schematic illustration of example barcoded oligonucleotide structure for use in analysis of RNA.

FIGS. 13A-C provides illustrations of example yields from template switch reverse transcription and PCR in partitions.

FIGS. 14A-B provides illustrations of example yields from reverse transcription and cDNA amplification in partitions with various cell numbers.

FIG. 15 provides an illustration of example yields from cDNA synthesis and real-time quantitative PCR at various input cell concentrations and also the effect of varying primer concentration on yield at a fixed cell input concentration.

FIG. 16 provides an illustration of example yields from in vitro transcription.

FIG. 17 shows an example computer control system that is programmed or otherwise configured to implement methods provided herein.

FIG. 18 shows an alignment of 3′ UTRs of ACD gene (top panel: Jurkat:293T 1:1 mixing sample; middle panel: Jurkat sample; bottom panel: 293T sample). Library insert size is ˜400 nt on average.

FIG. 19 is an illustration of a SNP at position 1890 of ACD transcript. The reference allele is ‘T’. In the Jurkat sample (middle), the alignment shows an alternative allele of ‘C.’ In the mixed sample (top panel), there is approximately a 1:1 mix of ‘C’ and ‘T’ at the position 1890. FIG. 19 discloses SEQ ID NOS 37 and 38, respectively, in order of appearance

FIGS. 20A-20D illustrate the presence of species specific single nucleotide polymorphisms (SNPs). FIG. 20A shows the distribution of Jurkat-specific SNPs in a Jurkat sample. FIG. 20B shows the distribution of 293T-specific SNPs in a 293T sample. FIG. 20C shows the distribution of Jurkat-specific and 293T-specific SNPs in a Jurkat:293T mixing sample. FIG. 20D shows that Jurkat and 293T cells can be separated by a Jurkat-specific marker gene, CD3D.

FIGS. 21A-21F illustrate the workflow for 3′ profiling of RNAs from thousands of single cells simultaneously. FIG. 21A illustrates an scRNA-seq workflow using the methods and systems described herein. FIG. 21B illustrates schematically the formation of GEMs by combining cells and reagents in one channel of a microfluidic chip with gel beads from another channel and subsequent mixing with oil-surfactant solution at a microfluidic junction. Single-cell GEMs were collected in the GEM outlet. FIG. 21C shows the percentage of GEMs containing 0, 1, or >1 gel beads (N=0, N=1, or N>1). Results are from five independent runs from multiple chip and gel bead lots over >70 k GEMs for each run, n=5, mean±s.e.m. FIG. 21D illustrates schematically a barcoded oligonucleotide comprising Illumina adapters, barcode sequences, unique molecular identifier (UMI) sequence and oligo dTs, which can prime reverse transcription of polyadenylated RNAs. FIG. 21D disclose SEQ ID NO:1. FIG. 21E illustrates schematically a finished library molecule comprising Illumina adapters and sample indices, allowing pooling and sequencing of multiple libraries on a next-generation short read sequencer. FIG. 21E discloses SEQ ID NO: 1. FIG. 21F illustrates schematically pipeline workflow for sequencing data analysis. The bottom box is an output of the pipeline.

FIGS. 22A-22X demonstrate an application of methods and systems disclosed herein for analyzing cell lines and External RNA Controls Consortium (ERCC). FIG. 22A shows a scatter plot of human and mouse UMI counts detected in a mixture of 293T and 3T3 cells. Cell barcodes containing primarily mouse reads aligned with the vertical axis and are termed ‘Mouse-only’; cell barcodes with primarily human reads aligned along the horizontal axis and are termed ‘Human-only’; and cell barcodes with significant mouse and human reads are not aligned with either the horizontal or vertical axis and are termed ‘Human:Mouse’. FIG. 22B shows the inferred multiplet rate as a function of recovered cell number. FIG. 22C shows the expected (Poisson sampling) and observed (manual counting) number of cells per GEM. Ncell, number of cells in each GEM. FIGS. 22D and 22E show the median number of genes and UMI counts, respectively, detected per cell in a mixture of 293T and 3T3 cells at different raw reads per cell. Data from three independent experiments were included, mean±s.e.m. FIG. 22F shows UMI count distribution of 293T cells (left), and 3T3 cells (right) in the 293T and 3T3 cell mixing sample. FIG. 22G shows CV and CV² of UMIs from 293Ts and 3T3s of 4 independent experiments. FIGS. 22H and 22I show the distribution of normalized UMI counts vs. GC content and gene length in 293T cells, respectively. UMI counts were normalized by RNA content. FIGS. 22J and 22K show the distribution of normalized UMI counts vs. GC content and gene length in 3T3 cells. Only genes with at least 1 UMI count detected in at least 1 cell were used. UMI normalization was performed by first dividing UMI counts by the total UMI counts in each cell, followed by multiplication with the median of the total UMI counts across cells. If there are multiple transcripts for a gene, the maximum length of the transcripts was used. Mean of GC content was calculated for each gene. FIG. 22L shows a comparison of the mean observed UMI counts for each ERCC molecule and the expected number of ERCC molecules per GEM. A straight line was fitted to summarize the relationship. FIG. 22M shows the distribution of Pearson correlation coefficient between expected vs. observed UMI counts for all GEMs, mean=0.94, sd=0.005. FIG. 22N shows the expected ERCC molecules per GEM vs. observed UMI counts at ERCC2 dilution of 1:50. FIG. 22O shows the conversion efficiency of each ERCC molecule as a function of their transcript GC content. FIG. 22P shows the conversion efficiency of each ERCC molecule as a function of their transcript length. FIG. 22Q shows the conversion efficiency estimated from ddPCR assay of 8 genes. FIG. 22R shows CV² vs. mean UMI counts, where CV is the coefficient of variation, defined as the ratio of the standard deviation to the mean (on a log-log scale). The dashed line represents CV²=1/mean. FIG. 22S illustrates schematically secondary analysis—automatic (left) and custom (right)—performed in methods disclosed herein. FIG. 22T shows the results from principal component analysis performed on normalized scRNA-seq data of Jurkat and 293T cells mixed at four different ratios (100% 293T, 100% Jurkat, 50:50 293T:Jurkat and 1:99 293T and Jurkat). PCI and PC3 are plotted, and each cell is colored by the normalized expression of CD3D. FIG. 22U shows that the expected cell proportion is well correlated with observed cell proportion among 12 independent experiments. FIG. 22V shows principal component 1 vs. 3 of normalized scRNA-seq data, with each cell colored by normalized expression of XIST. FIG. 22W shows the distribution of filtered SNVs/cell in 293Ts. FIG. 22X provides plots showing 293T- and Jurkat-enriched SNVs. A 3.1% multiplet rate was inferred from the 50:50 293T:Jurkat sample.

FIGS. 23A-23Q illustrate subpopulation discovery from a large immune population. FIG. 23A shows the distribution of number of genes (left) and UMI counts (right) detected per 68 k PBMCs. FIG. 23B shows median number of genes (left) and UMI counts (right) detected per cell as a function of raw reads per cell. FIG. 23C shows total RNA (pg/cell) in PBMCs, 293Ts and 3T3s. (n=7 for PBMC, n=4 for 293T, n=4 for 3T3 cells, mean±s.e.m.). FIG. 23D shows normalized dispersion vs. mean UMI counts. Black dots represent top most variable genes used for PCA. FIG. 23E shows tSNE projection of 68 k PBMCs, where each cell is grouped into one of the 10 clusters (distinguished by their colours). Cluster number is indicated, with the percentage of cells in each cluster noted within parentheses. FIG. 23F shows within groups sum of squares vs. number of clusters for k-means clustering. FIG. 23G shows normalized expression (centered) on the top variable genes (rows) from each of 10 clusters (columns) in a heat map. Numbers at the top indicate cluster number in FIG. 23E, with connecting lines indicating the hierarchical relationship between clusters. Representative markers from each cluster are shown on the right, and an inferred cluster assignment is shown on the left. FIGS. 23H-23J and 23N-23P show tSNE projection of 68 k PBMCs, with each cell coloured based on their normalized expression of CD3D, CD8A, NKG7, FCER1A, CD16, and A100A8. UMI normalization was performed by first dividing UMI counts by the total UMI counts in each cell, followed by multiplication with the median of the total UMI counts across cells. Then, the natural log of the UMI counts was taken. Finally, each gene was normalized such that the mean signal for each gene was 0, and standard deviation was 1. FIGS. 23K-23M and 23Q show tSNE projection of 68 k PBMCs, coloured by normalized expression of CD79A, CD4, CCR10 and PF4 in each cell, respectively. UMI normalization was performed by first dividing UMI counts by the total UMI counts in each cell, followed by multiplication with the median of the total UMI counts across cells. Then, the natural log of UMI counts was taken. Finally, each gene was normalized such that the mean signal for each gene was 0, and the standard deviation was 1.

FIGS. 24A-24W further illustrate the ability to detect distinct populations in fresh 68 k PBMCs. FIGS. 24A-24J show FACS analysis of bead enriched sub-populations of PBMCs. FIG. 24K provides a heatmap displaying the correlation coefficient in pairwise comparison of 11 purified sub-populations of PBMCs. Correlation was calculated using their average expression profile and grouped by hierarchical clustering. FIGS. 24L-24U show tSNE projections for each purified population. In FIGS. 24L, 24R, 24T and 24U, each cell is colored by normalized expression of marker genes FTL, CLEC9A, CD8A, CD34 and CD27 respectively. UMI normalization was performed by first dividing UMI counts by the total UMI counts in each cell, followed by multiplication with the median of the total UMI counts across cells. Then, the natural log of the UMI counts was taken. Finally, each gene was normalized such that the mean signal for each gene was 0, and standard deviation was 1. When more than 1 population was detected in a sample, e.g., FIGS. 24L and 24T, only the population showing the correct marker expression was selected (marked by a dotted polygon). FIG. 24V shows tSNE projection of 68 k PBMCs, with each cell coloured based on their correlation-based assignment to a purified subpopulation of PBMCs. Subclusters within T cells are marked by dashed polygons. NK, natural killer cells; reg T, regulatory T cells. FIG. 24W shows Seurat's tSNE projection of 68 k PBMCs, coloured by the inferred cell type assignment from purified PBMCs.

FIGS. 25A-25C compare the differences between fresh and frozen PBMCs from Donor A. FIG. 25A shows a scatterplot of mean UMI counts per gene across all cells between fresh vs. matched frozen PBMCs. Red dots represent genes that show 2-fold upregulation in frozen PBMCs. FIG. 25B shows median genes (left) and UMI counts (right) detected per cell between fresh and frozen PBMCs (n=3). Black points correspond to fresh PBMCs, whereas grey points correspond to frozen PBMCs. Wilcoxon ranksum test was used to test whether the number of genes and UMI counts from fresh and frozen PBMCs were significantly different. FIG. 25C shows the proportion of major cell types detected in fresh and frozen PBMCs (n=3).

FIGS. 26A-26H illustrate SNV analysis of scRNA-seq data. FIG. 26A shows the distribution of filtered SNVs in each PBMC from Donor B. FIG. 26B shows the distribution of filtered SNVs in each PBMC from Donor C. FIG. 26C shows sensitivity versus percentage of minor population, where sensitivity is evaluated against the true labeling of in silico mixed PBMCs from Donors B and C. Red line indicates that the major population comes from Donor B PBMCs. Blue line indicates that the major population comes from Donor C PBMCs. FIG. 26D shows positive predictive value (PPV) versus percentage of minor population, where PPV is evaluated against the true labeling of in silico mixed PBMCs from Donors B and C. Red line indicates that the major population comes from Donor B PBMCs. Blue line indicates that the major population comes from Donor C PBMCs. FIG. 26E shows called mix fraction versus actual mix fraction in in silico mixing of PBMCs from Donors B and C. Fifty percent actual mix fraction is correctly called (not shown). FIG. 26F shows % minor populations that can be confidently detected (PPV and sensitivity >0.95) vs. base error rate. FIG. 26G shows tSNE projection of PBMCs from Donor B and Donor C in 50:50 PBMC B:C sample, where each cell is colored based on their clustering (k-means) assignment. FIG. 26H compares expression between 5 clusters of PBMCs from Donors B and C, with red indicating high similarity and blue indicating lower similarity. 100 cells were sampled from each cluster of PBMCs from Donors B and C, and their pairwise gene expression was compared against each other.

FIGS. 27A-27H shows the results from analysis of transplant samples. FIG. 27A shows median number of genes (left) and UMIs (right) detected per cell for pre-transplant, post-transplant and BMMCs from 2 healthy donors. FIG. 27B shows distribution of filtered SNV counts per cell in AML027 pre-transplant sample. FIG. 27C shows distribution of filtered SNV counts per cell in AML035 pre-transplant sample. FIG. 27D shows tSNE projection of scRNA-seq data from a healthy control, AML027 pre- and post-transplant samples (post-transplant sample is separated into host and donor) and AML035 pre- and post-transplant samples. tSNE projection was also performed on a second healthy control (not shown). Each cell is coloured by their classification, which is labelled next to the cell clusters. FIG. 27E shows tSNE projection of pooled 6 samples (2 healthy donors, 2 AML027 host and 2AML035), colored by k-means clustering assignment. FIG. 27F shows normalized expression (centered) of the top variable genes (rows) from each of 9 clusters (columns) in a heatmap. Numbers on the right side indicate cluster number in FIG. 27E, with connecting lines indicating the hierarchical relationship between clusters. Representative markers from each cluster are shown on the top. FIG. 27G shows tSNE projection of all cells, with each cell colored by normalized expression of HBA1, AZU1, IL8, CD34, GATA1, and CD71 respectively. UMI normalization was performed by first dividing UMI counts by the total UMI counts in each cell, followed by multiplication with the median of the total UMI counts across cells. The natural log of the UMI counts was then taken. Finally, each gene was normalized such that the mean signal for each gene was 0, and standard deviation was 1. FIG. 27H shows the proportion of subpopulations in each sample.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “barcode,” as used herein, generally refers to a label, or identifier, that can be part of an analyte to convey information about the analyte. A barcode can be a tag attached to an analyte (e.g., nucleic acid molecule) or a combination of the tag in addition to an endogenous characteristic of the analyte (e.g., size of the analyte or end sequence(s)). The barcode may be unique. Barcodes can have a variety of different formats, for example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads in real time.

The term “subject,” as used herein, can be used interchangeably with “patient” and generally refers to an animal such as a mammal including, but not limited to, non-primates such as, for example, a cow, pig, horse, cat, dog, rat and mouse; and primates such as, for example, a monkey or a human. A subject can be a healthy individual, an individual that has or is suspected of having a disease or a pre-disposition to the disease, an individual that is in need of therapy or suspected of needing therapy, or an individual who is undergoing a therapy or a treatment for a disease or medical condition. In various embodiments, a subject comprises a cell sample for which analysis, e.g., transcriptome analysis, is desired.

The term “genome,” as used herein, generally refers to an entirety of a subject's hereditary information. A genome can be encoded either in DNA or in RNA. A genome can comprise coding regions that code for proteins as well as non-coding regions. A genome can include the sequence of all chromosomes together in an organism. For example, the human genome has a total of 46 chromosomes. The sequence of all of these together may constitute a human genome.

The term “sequencing,” as used herein, generally refers to methods and technologies for determining the sequence of nucleotide bases in one or more polynucleotides. The polynucleotides can be, for example, deoxyribonucleic acid (DNA) or ribonucleic acid (RNA), including variants or derivatives thereof (e.g., single stranded DNA). Sequencing devices may provide a plurality of sequence reads corresponding to the genetic information of a subject (e.g., human), as generated by the device from a sample comprising polynucleotides.

The term “genetic aberration,” as used herein, generally refers to a genetic variant, such as a nucleic acid molecule comprising a polymorphism. An aberration can be a structural variant or copy number variant, which can be genomic variants that are larger than single nucleotide variants or short indels. An aberration can be an alteration or polymorphism in a nucleic acid sample or genome of a subject. Single nucleotide polymorphisms (SNPs) are a form of polymorphisms. Polymorphisms can include single nucleotide variations (SNVs), insertions, deletions, repeats, small insertions, small deletions, small repeats, structural variant junctions, variable length tandem repeats, and/or flanking sequences. Copy number variants (CNVs), transversions and other rearrangements are also forms of genetic variation. A genomic alternation may be a base change, insertion, deletion, repeat, copy number variation, or transversion.

The term “bead,” as used herein, generally refers to a particle. The bead may be a solid or semi-solid particle. The bead may comprise a gel bead. The bead may be formed of a polymeric material. In some cases, the bead can be magnetic.

The term “sample,” as used herein, generally refers to a biological sample of a subject. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swap. The sample may be a plasma or serum sample. The sample may comprise cells. The cells of a sample, in some cases, is a homogeneous cell population, or of the same kind. Alternatively, the cells of a sample can be a heterogeneous cell population, or of different kinds or diverse in content. In some cases, nucleic acids or polynucleotides can be obtained from cells of a sample. The sample may be a cell-free sample. A cell-free sample may include extracellular polynucleotides. Extracellular polynucleotides may be isolated from a bodily sample that may be selected from a group consisting of blood, plasma, serum, urine, saliva, mucosal excretions, sputum, stool and tears.

### I. Single Cell Analysis

Advanced nucleic acid sequencing technologies have resulted in various accomplishments in sequencing biological materials, including providing substantial sequence information on individual organisms, and relatively pure biological samples. However, sub-populations of cells in biological samples that may represent a minority of the overall make-up of the sample can be overlooked by techniques which measure average values from a population. Information derived from single-cells, such as individualized sequence information, can be of significant value.

In various applications, nucleic acid sequencing technologies derive the nucleic acid molecules (used interchangeably with ‘nucleic acids’) that they sequence from collections of cells derived from a tissue sample or other biological sample. Cells from these samples can be processed, en masse, to extract the genetic material that represents an average of the population of cells, which can then be processed into sequencing ready DNA libraries that are configured for a given sequencing technology. Although often discussed in terms of DNA or nucleic acids, the nucleic acids derivable from the cells include, but are not limited to, DNA and RNA, including, e.g., mRNA, total RNA, or the like, that may be processed to produce cDNA for sequencing. When analyzing expression levels, e.g., of mRNA, an ensemble approach can, in some cases, be predisposed to presenting potentially inaccurate data from cell populations that are heterogenous in terms of expression levels. In some cases, where expression is high in a small minority of the cells in an analyzed population, and absent in the majority of the cells of the population, an ensemble method may indicate low level expression for the entire population.

This original majority bias can be further magnified through additional downstream sample preparation methods, for example, methods of generating sequencing libraries. In particular, next generation sequencing technologies may rely upon the geometric amplification of nucleic acid fragments, such as the polymerase chain reaction (PCR), in order to produce a sufficient amount of nucleic acid for a sequencing library. However, such geometric amplification can be biased toward amplification of majority constituents in a sample, and may not preserve the starting ratios of such minority and majority components. By way of example, if a sample includes 95% DNA from a particular cell type in a sample, e.g., host tissue cells, and 5% DNA from another cell type, e.g., cancer cells, PCR based amplification can preferentially amplify the majority DNA in place of the minority DNA, both as a function of comparative exponential amplification (the repeated doubling of the higher concentration quickly outpaces that of the smaller fraction) and as a function of sequestration of amplification reagents and resources (as the larger fraction is amplified, it preferentially utilizes primers and other amplification reagents).

While some of these challenges can be addressed by utilizing different sequencing systems, such as single molecule systems that do not require amplification, the single molecule systems, as well as the ensemble sequencing methods of other next generation sequencing (NGS) systems, may have large input DNA requirements. For example, single molecule sequencing systems can have sample input DNA requirements of from 500 nanograms (ng) to upwards of 10 micrograms (μg). Likewise, other NGS systems can be optimized for starting amounts of sample DNA in the sample of from approximately 50 ng to about 1 μg.

### II. Compartmentalization and Characterization of Cells

Methods and systems provided herein can be used for characterizing nucleic acids at a single-cell level. In particular, the methods and systems described herein provide a droplet based system that enables 3′ mRNA digital counting of up to tens of thousands of single cells. In some embodiments, the methods described herein provide a droplet based system that enables 3′ mRNA digital counting of up to hundreds of thousands of single cells, up to millions of single cells, or more.

In an aspect, the methods and systems described herein enable single cell analysis utilizing compartmentalization or partitioning of individual cells into discrete compartments or partitions (used interchangeably). A whole cell can be isolated in a compartment, thereby, allowing that cell to remain separate from other cells of the sample. When desired, the nucleic acids from a whole cell can be released into the compartment, for example, by contacting the cell with a lysis agent or other stimulus. The released nucleic acids can remain in the compartment, separated from other cells of the sample and also the nucleic acids associated with other cells of the sample. Unique identifiers, e.g., barcodes, may be previously, subsequently or concurrently delivered to the compartments that hold single cells, in order to allow for the later attribution of, e.g., sequence information, to a particular cell. While in the partitions, unique identifiers, e.g., barcodes or barcode sequences, can be associated with the nucleic acid sequences of nucleic acids from the whole cell using various processes, including ligation and/or amplification techniques. These barcode sequences can be used to determine the origin of a nucleic acid and/or to identify various nucleic acid sequences as being associated with a particular cell. Such identification can then allow that analysis to be attributed back to the individual cell or small group of cells from which the nucleic acids were derived. This can be accomplished regardless of whether the cell population represents a 50/50 mix of cell types, a 90/10 mix of cell types, or virtually any ratio of cell types, as well as a complete heterogeneous mix of different cell types, or any mixture between these. Differing cell types may include cells or biologic organisms from different tissue types of an individual, from different individuals, from differing genera, species, strains, variants, or any combination of any or all of the foregoing. For example, differing cell types may include normal and tumor tissue from an individual, cells from a donor and a recipient (e.g., transplant), multiple different bacterial species, strains and/or variants from environmental, forensic, microbiome or other samples, or any of a variety of other mixtures of cell types.

In various embodiments, compartments comprise droplets of aqueous fluid within a non-aqueous continuous phase, e.g., an oil phase. In alternative embodiments, compartments can refer to containers or vessels (such as wells, microwells, tubes, through ports in nanoarray substrates, or other containers). These compartments may comprise, e.g., microcapsules or micro-vesicles that have an outer barrier surrounding an inner fluid center or core, or they may be a porous matrix that is capable of entraining and/or retaining materials within its matrix. A variety of different vessels are described in, for example, U.S. Patent Application Publication No. 20140155295, the full disclosure of which is incorporated herein by reference in its entirety for all purposes. Likewise, emulsion systems for creating stable droplets in non-aqueous or oil continuous phases are described in detail in, e.g., U.S. Patent Application Publication No. 20100105112, the full disclosure of which is incorporated herein by reference in its entirety for all purposes.

In the case of droplets in an emulsion, allocating individual cells to discrete compartments may generally be accomplished by introducing a flowing stream of cells in an aqueous fluid into a flowing stream of a non-aqueous fluid, such that droplets are generated at the junction of the two streams. By providing the aqueous cell-containing stream at a certain concentration level of cells, the level of occupancy of the resulting partitions in terms of numbers of cells can be controlled. In some cases, where single cell partitions are desired, it may be desirable to control the relative flow rates of the fluids such that, on average, the partitions contain less than one cell per partition, in order to ensure that those partitions which are occupied, are primarily singly occupied. The flow rate can also be altered to provide a higher percentage of partitions that are occupied, e.g., allowing for only a small percentage of unoccupied partitions. In some aspects, the flows and channel architectures are controlled as to ensure a desired number of singly occupied partitions, less than a certain level of unoccupied partitions and/or less than a certain level of multiply occupied partitions.

A droplet based system disclosed herein can capture any suitable percentage of a cell population to be analyzed into compartments, e.g., droplets. In some cases, it is desirable to capture the entire cell population into droplets. In other cases, capture of a percentage of the cell population is desired or sufficient for downstream analysis and assay. In some embodiments, at least about 5%, 10%, 15%, 20%, 25%, 30%, 35%, 40%, 45%, 50%, 55%, 60%, 65%, 70%, 75%, 80%, 85%, 90%, or 95% of the cells of a cell sample are captured in a droplet using a droplet based system provided herein. In some embodiments, at most about 5%, 10%, 15%, 20%, 25%, 30%, 35%, 40%, 45%, 50%, 55%, 60%, 65%, 70%, 75%, 80%, 85%, 90%, or 95% of the cells of a cell sample are captured in a droplet using a droplet based system provided herein. In some embodiments, approximately 5%, 10%, 15%, 20%, 25%, 30%, 35%, 40%, 45%, 50%, 55%, 60%, 65%, 70%, 75%, 80%, 85%, 90%, or 95% of the cells of a cell sample are captured in a droplet using a droplet based system provided herein. In some embodiments, between about 10% and about 95%, between about 15% and about 90%, between about 20% and about 85%, between about 25% and about 80%, between about 30% and about 75%, between about 35% and about 70%, between about 40% and about 65%, between about 45% and about 60%, or between about 50% and about 55% of cells of a cell sample are captured in a droplet using a droplet based system provided herein. In some embodiments, the percentage of cells captured into droplets can be optimized for a particular type of assay. In some embodiments, approximately 50% of cells of a cell sample loaded into a droplet based system are captured in a droplet.

In many cases, a substantial majority of occupied partitions (partitions containing one or more microcapsules) formed from methods and systems disclosed herein include no more than 1 cell per occupied partition. In some cases, fewer than 25% of the occupied partitions contain more than one cell, and in many cases, fewer than 20% of the occupied partitions have more than one cell, while in some cases, fewer than 10% or even fewer than 5% of the occupied partitions include more than one cell per partition.

Additionally or alternatively, in many cases, it is desirable to avoid the creation of excessive numbers of empty partitions. While this may be accomplished by providing sufficient numbers of cells into the partitioning zone, the Poissonian distribution would expectedly increase the number of partitions that would include multiple cells. In some embodiments, the flow of one or more of the cells, or other fluids directed into the partitioning zone are such that, in many cases, no more than 50% of the generated partitions, 25% of partitions, or 10% of partitions are unoccupied (e.g., including less than 1 cell). Further, in some aspects, these flows are controlled so as to present non-Poissonian distribution of single occupied partitions while providing lower levels of unoccupied partitions.

Although described in terms of providing substantially singly occupied partitions, above, in certain cases, it is desirable to provide multiply occupied partitions, e.g., containing two, three, four or more cells within a single partition. Accordingly, as noted above, the flow characteristics of the cell and/or bead containing fluids and partitioning fluids may be controlled to provide for such multiply occupied partitions. In particular, the flow parameters may be controlled to provide a desired occupancy rate at greater than 50% of the partitions, greater than 75%, and in some cases greater than 80%, 85%, 90%, 95%, or higher.

The partitions described herein can be characterized by having extremely small volumes, e.g., less than 10 microliters (μL), 5 μL, 1 μL, 900 nanoliters (nL), 500 nL, 100 nL, 50 nL, 1 nL, 900 picoliters (pL), 800 pL, 700 pL, 600 pL, 500 pL, 400 pL, 300 pL, 200 pL, 100 pL, 50 pL, 20 pL, 10 pL, or 1 pL. For example, in the case of droplet based partitions, the droplets may have overall volumes that are less than 1000 pL, 900 pL, 800 pL, 700 pL, 600 pL, 500 pL, 400 pL, 300 pL, 200 pL, 100 pL, 50 pL, 20 pL, 10 pL, or even less than 1 pL. Where co-partitioned with beads, it will be appreciated that the sample fluid volume, e.g., including co-partitioned cells, within the partitions may be less than 90% of the above described volumes, less than 80%, less than 70%, less than 60%, less than 50%, less than 40%, less than 30%, less than 20%, or even less than 10% the above described volumes.

Multiple samples can be processed in parallel using droplet based systems disclosed herein. In some embodiments, at least 2, 3, 4, 5, 6, 7, 8, 9, or 10 samples are processed in parallel. The multiple samples processed in parallel may comprise similar numbers of cells. In some cases, the multiple samples processed in parallel do not comprise similar numbers of cells.

A cell population for analysis can comprise any number of cells. In some embodiments, a cell sample loaded on a droplet based system of the disclosure comprises at least about 100, 1,000, 10,000, 20,000, 30,000, 40,000, 50,000, 60,000, 70,000, 80,000, 90,000, 100,000, 125,000, 150,000, 175,000, 200,000, 225,000, 250,000, 275,000, 300,000, 325,000, 350,000, 375,000, 400,000, 425,000, 450,000, 475,000, 500,000, 525,000, 550,000, 575,000, 600,000, 625,000, 650,000, 675,000, 700,000, 725,000, 750,000, 775,000, 800,000, 825,000, 850,000, 875,000, 900,000, 925,000, 950,000, 975,000, or 1,000,000 cells. In some embodiments, a cell sample loaded on a droplet based system of the disclosure comprises at most about 100, 1,000, 10,000, 20,000, 30,000, 40,000, 50,000, 60,000, 70,000, 80,000, 90,000, 100,000, 125,000, 150,000, 175,000, 200,000, 225,000, 250,000, 275,000, 300,000, 325,000, 350,000, 375,000, 400,000, 425,000, 450,000, 475,000, 500,000, 525,000, 550,000, 575,000, 600,000, 625,000, 650,000, 675,000, 700,000, 725,000, 750,000, 775,000, 800,000, 825,000, 850,000, 875,000, 900,000, 925,000, 950,000, 975,000, or 1,000,000 cells. In some embodiments, a cell sample loaded on a droplet based system of the disclosure comprises approximately 100, 1,000, 10,000, 20,000, 30,000, 40,000, 50,000, 60,000, 70,000, 80,000, 90,000, 100,000, 125,000, 150,000, 175,000, 200,000, 225,000, 250,000, 275,000, 300,000, 325,000, 350,000, 375,000, 400,000, 425,000, 450,000, 475,000, 500,000, 525,000, 550,000, 575,000, 600,000, 625,000, 650,000, 675,000, 700,000, 725,000, 750,000, 775,000, 800,000, 825,000, 850,000, 875,000, 900,000, 925,000, 950,000, 975,000, or 1,000,000 cells.

As is described elsewhere herein, partitioning species may generate a population of partitions. In such cases, any suitable number of partitions can be generated to generate the population of partitions. For example, in a method described herein, a population of partitions may be generated that comprises at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions or at least about 1,000,000,000 partitions. Moreover, the population of partitions may comprise both unoccupied partitions (e.g., empty partitions) and occupied partitions.

### III. Barcodes

Unique identifiers, e.g., barcodes, may be previously, subsequently or concurrently delivered to the partitions that hold the compartmentalized or partitioned cells. Barcodes, which comprise a barcode sequence, may be delivered, in some embodiments, on an oligonucleotide (referred to interchangeably as a “barcoded oligonucleotide” or “oligonucleotide barcode”), to a partition via any suitable mechanism.

In some embodiments, barcoded oligonucleotides are delivered to a partition via a microcapsule. In some cases, barcoded oligonucleotides are initially associated with the microcapsule and then released from the microcapsule upon application of a stimulus which allows the oligonucleotides to dissociate or to be released from the microcapsule.

A microcapsule, in some embodiments, comprises a bead. In some embodiments, a bead may be porous, non-porous, solid, semi-solid, semi-fluidic, or fluidic. In some embodiments, a bead may be dissolvable, disruptable, or degradable. In some cases, a bead may not be degradable. In some embodiments, the bead may be a gel bead. A gel bead can be a hydrogel bead. A gel bead can be formed from molecular precursors, such as a polymeric or monomeric species. A semi-solid bead can be a liposomal bead. Solid beads can comprise metals including iron oxide, gold, and silver. In some cases, the beads are silica beads. In some cases, the beads are rigid. In some cases, the beads are flexible and/or compressible.

The beads may contain molecular precursors (e.g., monomers or polymers), which may form a polymer network via polymerization of the precursors. In some cases, a precursor may be an already polymerized species capable of undergoing further polymerization via, for example, a chemical cross-linkage. In some cases, a precursor comprises one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers.

A bead may comprise natural and/or synthetic materials. For example, a polymer can be a natural polymer or a synthetic polymer. In some cases, a bead comprises both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and combinations (e.g., co-polymers) thereof. Beads may also be formed from materials other than polymers, including lipids, micelles, ceramics, glass-ceramics, material composites, metals, other inorganic materials, and others.

In some cases, a chemical cross-linker may be a precursor used to cross-link monomers during polymerization of the monomers and/or may be used to attach oligonucleotides (e.g., barcoded oligonucleotides) to the bead. In some cases, polymers may be further polymerized with a cross-linker species or other type of monomer to generate a further polymeric network. Non-limiting examples of chemical cross-linkers (also referred to as a “crosslinker” or a “crosslinker agent” herein) include cystamine, gluteraldehyde, dimethyl suberimidate, N-Hydroxysuccinimide crosslinker BS3, formaldehyde, carbodiimide (EDC), SMCC, Sulfo-SMCC, vinylsilane, N,N′diallyltartardiamide (DATD), N,N′-Bis(acryloyl)cystamine (BAC), or homologs thereof. In some cases, the crosslinker used in the present disclosure contains cystamine.

Crosslinking may be permanent or reversible, depending upon the particular crosslinker used. Reversible crosslinking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some embodiments, disulfide linkages can be formed between molecular precursor units (e.g., monomers, oligomers, or linear polymers) or precursors incorporated into a bead and oligonucleotides. Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some embodiments, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some embodiments, the bead may comprise covalent or ionic bonds between polymeric precursors (e.g., monomers, oligomers, linear polymers), oligonucleotides, primers, and other entities. In some cases, the covalent bonds comprise carbon-carbon bonds or thioether bonds.

In some cases, a bead may comprise an acrydite moiety, which in certain aspects may be used to attach one or more oligonucleotides (e.g., barcode sequence, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as an oligonucleotide (e.g., barcode sequence, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment is reversible, such that when the disulfide bond is broken (e.g., in the presence of a reducing agent), the attached species is released from the bead. In other cases, an acrydite moiety comprises a reactive hydroxyl group that may be used for attachment.

Functionalization of beads for attachment of oligonucleotides may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to an oligonucleotide, such as a primer (e.g., a primer for amplifying target nucleic acids, barcoded oligonucleotide, etc) that is desired to be incorporated into the bead. In some cases, the primer comprises a P5 sequence for attachment to a sequencing flow cell for Illumina sequencing. In some cases, the primer comprises a P7 sequence for attachment to a sequencing flow cell for Illumina sequencing. In some cases, the primer comprises a barcode sequence. In some cases, the primer further comprises a unique molecular identifier (UMI). In some cases, the primer comprises an R1 primer sequence for Illumina sequencing. In some cases, the primer comprises an R2 primer sequence for Illumina sequencing.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than about 10,000, 100,000, 1,000,000, 10,000,000, 100,000,000, 1,000,000,000, 10,000,000,000, or 100,000,000,000) of reducing agent may be used for reduction. Controlling the number of disulfide linkages that are reduced to free thiols may be useful in ensuring bead structural integrity during functionalization. In some cases, optically-active agents, such as fluorescent dyes may be may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

In some cases, an acrydite moiety linked to precursor, another species linked to a precursor, or a precursor itself comprises a labile bond, such as chemically, thermally, or photo-sensitive bonds e.g., disulfide bonds, UV sensitive bonds, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)).

Species that do not participate in polymerization may also be encapsulated in beads during bead generation (e.g., during polymerization of precursors). Such species may be entered into polymerization reaction mixtures such that generated beads comprise the species upon bead formation. In some cases, such species may be added to the beads after formation. Such species may include, for example, oligonucleotides, reagents for a nucleic acid amplification reaction (e.g., primers, polymerases, dNTPs, co-factors (e.g., ionic co-factors)) including those described herein, reagents for enzymatic reactions (e.g., enzymes, co-factors, substrates), or reagents for a nucleic acid modification reactions such as polymerization, ligation, or digestion. Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead.

Beads may be of uniform size or heterogeneous size. In some cases, the diameter of a bead may be about 1 μm, 5 μm, 10 μm, 20 μm, 30 μm, 40 μm, 50 μm, 60 μm, 70 μm, 80 μm, 90 μm, 100 μm, 250 μm, 500 μm, or 1 mm. In some cases, a bead may have a diameter of at least about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or more. In some cases, a bead may have a diameter of less than about 1 μm, 5 μm, 10 μm, 20 μm, 30 μm, 40 μm, 50 μm, 60 μm, 70 μm, 80 μm, 90 μm, 100 μm, 250 μm, 500 μm, or 1 mm. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads are provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, or even less than 5%.

Beads may be of any suitable shape. Examples of bead shapes include, but are not limited to, spherical, non-spherical, oval, oblong, amorphous, circular, cylindrical, and variations thereof.

In addition to, or as an alternative to the cleavable linkages between the beads and the associated molecules, e.g., barcode containing oligonucleotides, described above, the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead is degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a oligonucleotide, e.g., barcoded oligonucleotide) may result in release of the species from the bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., an oligonucleotide, a barcode sequence, a primer, etc) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent breaks the various disulfide bonds resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be desirable to avoid exposing such beads to the stimulus or stimuli that cause such degradation prior to the desired time, in order to avoid premature bead degradation and issues that arise from such degradation, including for example poor flow characteristics and aggregation. By way of example, where beads comprise reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than 1/10th, less than 1/50th, and even less than 1/100th of the lower ranges for such materials used in degrading the beads. For example, for DTT, the reducing agent free preparation will typically have less than 0.01 mM, 0.005 mM, 0.001 mM DTT, 0.0005 mM DTT, or even less than 0.0001 mM DTT. In many cases, the amount of DTT will be undetectable.

In some cases, a stimulus may be used to trigger degradation of the bead, which may result in the release of contents from the bead. Generally, a stimulus may cause degradation of the bead structure, such as degradation of the covalent bonds or other types of physical interaction. These stimuli may be useful in inducing a bead to degrade and/or to release its contents. Examples of stimuli that may be used include chemical stimuli, thermal stimuli, optical stimuli (e.g., light) and any combination thereof, as described more fully below.

Numerous chemical triggers may be used to trigger the degradation of beads. Examples of these chemical changes may include, but are not limited to pH-mediated changes to the integrity of a component within the bead, degradation of a component of a bead via cleavage of cross-linked bonds, and depolymerization of a component of a bead.

In some embodiments, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

The methods, compositions, devices, and kits of this disclosure may be used with any suitable agent to degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, or 10 mM. The reducing agent may be present at a concentration of at least about 0.1 mM, 0.5 mM, 1 mM, 5 mM, 10 mM, or greater. The reducing agent may be present at concentration of at most about 0.1 mM, 0.5 mM, 1 mM, 5 mM, or 10 mM.

Any suitable number of nucleic acid molecules (e.g., primer, e.g., barcoded oligonucleotide) can be associated with a bead such that, upon release from the bead, the nucleic acid molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer is limited by the process of producing oligonucleotide bearing beads.

Additionally, in many cases, the multiple beads within a single partition may comprise different reagents associated therewith. In such cases, it may be advantageous to introduce different beads into a common channel or droplet generation junction, from different bead sources, i.e., containing different associated reagents, through different channel inlets into such common channel or droplet generation junction. In such cases, the flow and frequency of the different beads into the channel or junction may be controlled to provide for the desired ratio of microcapsules from each source, while ensuring the desired pairing or combination of such beads into a partition with the desired number of cells.

### IV. Droplet Based Systems

In certain cases, microfluidic channel networks are particularly suited for generating partitions as described herein. Alternative mechanisms may also be employed in the partitioning of individual cells, including porous membranes through which aqueous mixtures of cells are extruded into non-aqueous fluids. Such systems are generally available from, e.g., Nanomi, Inc.

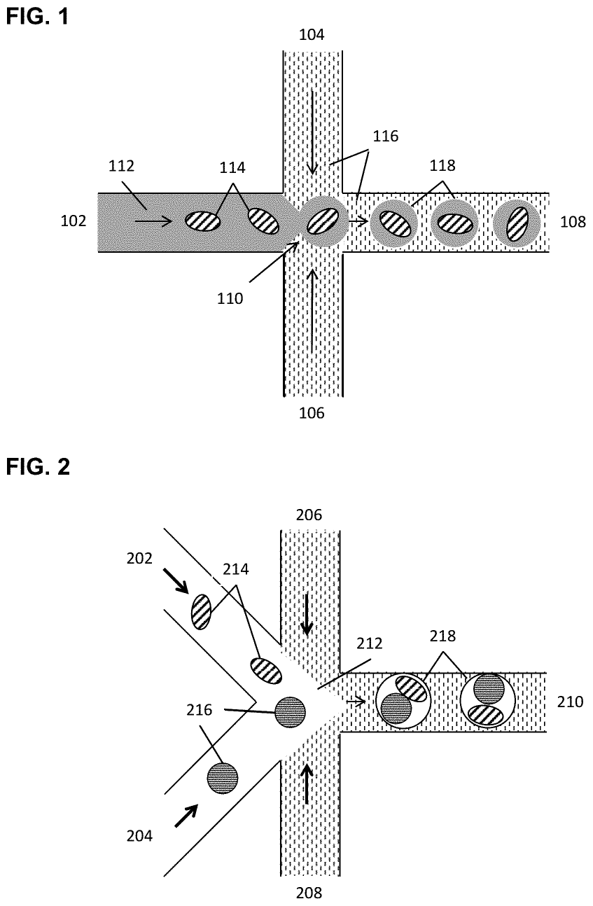

An example of a simplified microfluidic channel structure for partitioning individual cells is illustrated in FIG. 1. As described elsewhere herein, in some cases, the majority of occupied partitions include no more than one cell per occupied partition and, in some cases, some of the generated partitions are unoccupied. In some cases, though, some of the occupied partitions may include more than one cell. In some cases, the partitioning process may be controlled such that fewer than 25% of the occupied partitions contain more than one cell, and in many cases, fewer than 20% of the occupied partitions have more than one cell, while in some cases, fewer than 10% or even fewer than 5% of the occupied partitions include more than one cell per partition. As shown, the channel structure can include channel segments 102, 104, 106 and 108 communicating at a channel junction 110. In operation, a first aqueous fluid 112 that includes suspended cells 114, may be transported along channel segment 102 into junction 110, while a second fluid 116 that is immiscible with the aqueous fluid 112 is delivered to the junction 110 from channel segments 104 and 106 to create discrete droplets 118 of the aqueous fluid including individual cells 114, flowing into channel segment 108.

In some aspects, this second fluid 116 comprises an oil, such as a fluorinated oil, that includes a fluorosurfactant for stabilizing the resulting droplets, e.g., inhibiting subsequent coalescence of the resulting droplets. Examples of particularly useful partitioning fluids and fluorosurfactants are described for example, in U.S. Patent Application Publication No. 20100105112, the full disclosure of which is hereby incorporated herein by reference in its entirety for all purposes.

In other aspects, in addition to or as an alternative to droplet based partitioning, cells may be encapsulated within a microcapsule that comprises an outer shell or layer or porous matrix in which is entrained one or more individual cells or small groups of cells, and may include other reagents. Encapsulation of cells may be carried out by a variety of processes. In general, such processes combine an aqueous fluid containing the cells to be analyzed with a polymeric precursor material that may be capable of being formed into a gel or other solid or semi-solid matrix upon application of a particular stimulus to the polymer precursor. Such stimuli include, e.g., thermal stimuli (either heating or cooling), photo-stimuli (e.g., through photo-curing), chemical stimuli (e.g., through crosslinking, polymerization initiation of the precursor (e.g., through added initiators), or the like.

Preparation of microcapsules comprising cells may be carried out by a variety of methods. For example, air knife droplet or aerosol generators may be used to dispense droplets of precursor fluids into gelling solutions in order to form microcapsules that include individual cells or small groups of cells. Likewise, membrane based encapsulation systems, such as those available from, e.g., Nanomi, Inc., may be used to generate microcapsules as described herein. In some aspects, microfluidic systems like that shown in FIG. 1 may be readily used in encapsulating cells as described herein. In particular, and with reference to FIG. 1, the aqueous fluid comprising the cells and the polymer precursor material is flowed into channel junction 110, where it is partitioned into droplets 118 comprising the individual cells 114, through the flow of non-aqueous fluid 116. In the case of encapsulation methods, non-aqueous fluid 116 may also include an initiator to cause polymerization and/or crosslinking of the polymer precursor to form the microcapsule that includes the entrained cells. Examples of particularly useful polymer precursor/initiator pairs include those described in U.S. Patent Application Publication No. 20140378345, the full disclosure of which is hereby incorporated herein by reference in their entireties for all purposes.

For example, in the case where the polymer precursor material comprises a linear polymer material, e.g., a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams in channel segments 104 and 106, which initiates the copolymerization of the acrylamide and BAC into a cross-linked polymer network or, hydrogel.

Upon contact of the second fluid stream 116 with the first fluid stream 112 at junction 110 in the formation of droplets, the TEMED may diffuse from the second fluid 116 into the aqueous first fluid 112 comprising the linear polyacrylamide, which will activate the crosslinking of the polyacrylamide within the droplets, resulting in the formation of the gel, e.g., hydrogel, microcapsules 118, as solid or semi-solid beads or particles entraining the cells 114. Although described in terms of polyacrylamide encapsulation, other ‘activatable’ encapsulation compositions may also be employed in the context of the methods and compositions described herein. For example, formation of alginate droplets followed by exposure to divalent metal ions, e.g., Ca2+, can be used as an encapsulation process using the described processes. Likewise, agarose droplets may also be transformed into capsules through temperature based gelling, e.g., upon cooling, or the like. As will be appreciated, in some cases, encapsulated cells can be selectively releasable from the microcapsule, e.g., through passage of time, or upon application of a particular stimulus, that degrades the microcapsule sufficiently to allow the cell, or its contents to be released from the microcapsule, e.g., into an additional partition, such as a droplet. For example, in the case of the polyacrylamide polymer described above, degradation of the microcapsule may be accomplished through the introduction of an appropriate reducing agent, such as DTT or the like, to cleave disulfide bonds that cross link the polymer matrix. See, e.g., U.S. Patent Application Publication No. 20140378345, the full disclosures of which are hereby incorporated herein by reference in their entirety for all purposes.

As will be appreciated, encapsulated cells or cell populations can provide certain potential advantages of being storable, and more portable than droplet based partitioned cells. Furthermore, in some cases, it may be desirable to allow cells to be analyzed to incubate for a select period of time, in order to characterize changes in such cells over time, either in the presence or absence of different stimuli. In such cases, encapsulation of individual cells may allow for longer incubation than simple partitioning in emulsion droplets, although in some cases, droplet partitioned cells may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. As alluded to above, the encapsulation of cells may constitute the partitioning of the cells into which other reagents are co-partitioned. Alternatively, encapsulated cells may be readily deposited into other partitions, e.g., droplets, as described above.

In accordance with certain aspects, the cells may be partitioned along with lysis reagents in order to release the contents of the cells within the partition. In such cases, the lysis agents can be contacted with the cell suspension concurrently with, or immediately prior to the introduction of the cells into the partitioning junction/droplet generation zone, e.g., through an additional channel or channels upstream of channel junction 110. Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the cells to cause the release of the cell's contents into the partitions. For example, in some cases, surfactant based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Similarly, lysis methods that employ other methods may be used, such as electroporation, thermal, acoustic or mechanical cellular disruption may also be used in certain cases, e.g., non-emulsion based partitioning such as encapsulation of cells that may be in addition to or in place of droplet partitioning, where any pore size of the encapsulate is sufficiently small to retain nucleic acid fragments of a desired size, following cellular disruption.

In addition to the lysis agents co-partitioned with the cells described above, other reagents can also be co-partitioned with the cells, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated cells, the cells may be exposed to an appropriate stimulus to release the cells or their contents from a co-partitioned microcapsule. For example, in some cases, a chemical stimulus may be co-partitioned along with an encapsulated cell to allow for the degradation of the microcapsule and release of the cell or its contents into the larger partition. In some cases, this stimulus may be the same as the stimulus described elsewhere herein for release of oligonucleotides from their respective bead or partition. In alternative aspects, this may be a different and non-overlapping stimulus, in order to allow an encapsulated cell to be released into a partition at a different time from the release of oligonucleotides into the same partition.

Additional reagents may also be co-partitioned with the cells, such as endonucleases to fragment the cell's DNA, DNA polymerase enzymes and dNTPs used to amplify the cell's nucleic acid fragments and to attach the barcode oligonucleotides to the amplified fragments. Additional reagents may also include reverse transcriptase enzymes, including enzymes with terminal transferase activity, primers and oligonucleotides, and switch oligonucleotides (also referred to herein as “switch oligos”) which can be used for template switching. In some cases, template switching can be used to increase the length of a cDNA. In one example of template switching, cDNA can be generated from reverse transcription of a template, e.g., cellular mRNA, where a reverse transcriptase with terminal transferase activity can add additional nucleotides, e.g., polyC, to the cDNA that are not encoded by the template, such, as at an end of the cDNA. Switch oligos can include sequences complementary to the additional nucleotides, e.g. polyG. The additional nucleotides (e.g., polyC) on the cDNA can hybridize to the sequences complementary to the additional nucleotides (e.g., polyG) on the switch oligo, whereby the switch oligo can be used by the reverse transcriptase as template to further extend the cDNA. Switch oligos may comprise deoxyribonucleic acids, ribonucleic acids, modified nucleic acids including locked nucleic acids (LNA), or any combination.

In some cases, the length of a switch oligo may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 nucleotides or longer.

In some cases, the length of a switch oligo may be at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the nucleic acids contained therein may be further processed within the partitions. In accordance with the methods and systems described herein, the nucleic acid contents of individual cells are generally provided with unique identifiers such that, upon characterization of those nucleic acids they may be attributed as having been derived from the same cell or cells. The ability to attribute characteristics to individual cells or groups of cells is provided by the assignment of unique identifiers specifically to an individual cell or groups of cells, which is another advantageous aspect of the methods and systems described herein. In particular, unique identifiers, e.g., in the form of nucleic acid barcodes are assigned or associated with individual cells or populations of cells, in order to tag or label the cell's components (and as a result, its characteristics) with the unique identifiers. These unique identifiers are then used to attribute the cell's components and characteristics to an individual cell or group of cells. In some aspects, this is carried out by co-partitioning the individual cells or groups of cells with the unique identifiers. In some aspects, the unique identifiers are provided in the form of oligonucleotides that comprise nucleic acid barcode sequences that may be attached to or otherwise associated with the nucleic acid contents of individual cells, or to other components of the cells, and particularly to fragments of those nucleic acids. The oligonucleotides are partitioned such that as between oligonucleotides in a given partition, the nucleic acid barcode sequences contained therein are the same, but as between different partitions, the oligonucleotides can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from 6 to about 20 or more nucleotides within the sequence of the oligonucleotides. In some cases, the length of a barcode sequence may be 6, 7, 8, 9, 10, 11, 12, 13, 14, 15, 16, 17, 18, 19, 20 nucleotides or longer. In some cases, the length of a barcode sequence may be at least 6, 7, 8, 9, 10, 11, 12, 13, 14, 15, 16, 17, 18, 19, 20 nucleotides or longer. In some cases, the length of a barcode sequence may be at most 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be 4, 5, 6, 7, 8, 9, 10, 11, 12, 13, 14, 15, 16 nucleotides or longer. In some cases, the barcode subsequence may be at least 4, 5, 6, 7, 8, 9, 10, 11, 12, 13, 14, 15, 16 nucleotides or longer. In some cases, the barcode subsequence may be at most 4, 5, 6, 7, 8, 9, 10, 11, 12, 13, 14, 15, 16 nucleotides or shorter.

The co-partitioned oligonucleotides can also comprise other functional sequences useful in the processing of the nucleic acids from the co-partitioned cells. These sequences include, e.g., targeted or random/universal amplification primer sequences for amplifying the genomic DNA from the individual cells within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Again, co-partitioning of oligonucleotides and associated barcodes and other functional sequences, along with sample materials is described in, for example, U.S. Patent Application Publication No. 20140378345 and U.S. Patent Application Publication No. 20140227684, the full disclosures of which are incorporated herein by reference in their entireties for all purposes. As will be appreciated other mechanisms of co-partitioning oligonucleotides may also be employed, including, e.g., coalescence of two or more droplets, where one droplet contains oligonucleotides, or microdispensing of oligonucleotides into partitions, e.g., droplets within microfluidic systems.

Briefly, in one example, beads are provided that each include large numbers of the above described oligonucleotides releasably attached to the beads, where all of the oligonucleotides attached to a particular bead will include the same nucleic acid barcode sequence, but where a large number of diverse barcode sequences are represented across the population of beads used. In particularly useful examples, gel beads are used as a solid support and delivery vehicle for the oligonucleotides into the partitions, as they are capable of carrying large numbers of oligonucleotide molecules, and may be configured to release those oligonucleotides upon exposure to a particular stimulus, as described elsewhere herein. In some cases, the population of beads will provide a diverse barcode sequence library that includes at least 1,000 different barcode sequences, at least 5,000 different barcode sequences, at least 10,000 different barcode sequences, at least at least 50,000 different barcode sequences, at least 100,000 different barcode sequences, at least 1,000,000 different barcode sequences, at least 5,000,000 different barcode sequences, or at least 10,000,000 different barcode sequences. Additionally, each bead can be provided with large numbers of oligonucleotide molecules attached. In particular, the number of molecules of oligonucleotides including the barcode sequence on an individual bead can be at least 1,000 oligonucleotide molecules, at least 5,000 oligonucleotide molecules, at least 10,000 oligonucleotide molecules, at least 50,000 oligonucleotide molecules, at least 100,000 oligonucleotide molecules, at least 500,000 oligonucleotides, at least 1,000,000 oligonucleotide molecules, at least 5,000,000 oligonucleotide molecules, at least 10,000,000 oligonucleotide molecules, at least 50,000,000 oligonucleotide molecules, at least 100,000,000 oligonucleotide molecules, and in some cases at least 1 billion oligonucleotide molecules.

Moreover, when the population of beads is partitioned, the resulting population of partitions can also include a diverse barcode library that includes at least 1,000 different barcode sequences, at least 5,000 different barcode sequences, at least 10,000 different barcode sequences, at least at least 50,000 different barcode sequences, at least 100,000 different barcode sequences, at least 1,000,000 different barcode sequences, at least 5,000,000 different barcode sequences, or at least 10,000,000 different barcode sequences. Additionally, each partition of the population can include at least 1,000 oligonucleotide molecules, at least 5,000 oligonucleotide molecules, at least 10,000 oligonucleotide molecules, at least 50,000 oligonucleotide molecules, at least 100,000 oligonucleotide molecules, at least 500,000 oligonucleotides, at least 1,000,000 oligonucleotide molecules, at least 5,000,000 oligonucleotide molecules, at least 10,000,000 oligonucleotide molecules, at least 50,000,000 oligonucleotide molecules, at least 100,000,000 oligonucleotide molecules, and in some cases at least 1 billion oligonucleotide molecules.

In some cases, it may be desirable to incorporate multiple different barcodes within a given partition, either attached to a single or multiple beads within the partition. For example, in some cases, a mixed, but known barcode sequences set may provide greater assurance of identification in the subsequent processing, e.g., by providing a stronger address or attribution of the barcodes to a given partition, as a duplicate or independent confirmation of the output from a given partition.

The oligonucleotides are releasable from the beads upon the application of a particular stimulus to the beads. In some cases, the stimulus may be a photo-stimulus, e.g., through cleavage of a photo-labile linkage that releases the oligonucleotides. In other cases, a thermal stimulus may be used, where elevation of the temperature of the beads environment will result in cleavage of a linkage or other release of the oligonucleotides form the beads. In still other cases, a chemical stimulus is used that cleaves a linkage of the oligonucleotides to the beads, or otherwise results in release of the oligonucleotides from the beads. Examples of this type of system are described in U.S. Patent Application Publication No. 20140155295 and U.S. Patent Application Publication No. 20140378345, the full disclosures of which are hereby incorporated herein by reference in their entireties for all purposes. In one case, such compositions include the polyacrylamide matrices described above for encapsulation of cells, and may be degraded for release of the attached oligonucleotides through exposure to a reducing agent, such as DTT.

In accordance with the methods and systems described herein, the beads including the attached oligonucleotides are co-partitioned with the individual cells, such that a single bead and a single cell are contained within an individual partition. As noted above, while single cell/single bead occupancy is the most desired state, it will be appreciated that multiply occupied partitions (either in terms of cells, beads or both), or unoccupied partitions (either in terms of cells, beads or both) will often be present. An example of a microfluidic channel structure for co-partitioning cells and beads comprising barcode oligonucleotides is schematically illustrated in FIG. 2. As described elsewhere herein, in some aspects, a substantial percentage of the overall occupied partitions will include both a bead and a cell and, in some cases, some of the partitions that are generated will be unoccupied. In some cases, some of the partitions may have beads and cells that are not partitioned 1:1. In some cases, it may be desirable to provide multiply occupied partitions, e.g., containing two, three, four or more cells and/or beads within a single partition. As shown, channel segments 202, 204, 206, 208 and 210 are provided in fluid communication at channel junction 212. An aqueous stream comprising the individual cells 214, is flowed through channel segment 202 toward channel junction 212. As described above, these cells may be suspended within an aqueous fluid, or may have been pre-encapsulated, prior to the partitioning process.

Concurrently, an aqueous stream comprising the barcode carrying beads 216, is flowed through channel segment 204 toward channel junction 212. A non-aqueous partitioning fluid 216 is introduced into channel junction 212 from each of side channels 206 and 208, and the combined streams are flowed into outlet channel 210. Within channel junction 212, the two combined aqueous streams from channel segments 202 and 204 are combined, and partitioned into droplets 218, that include co-partitioned cells 214 and beads 216. As noted previously, by controlling the flow characteristics of each of the fluids combining at channel junction 212, as well as controlling the geometry of the channel junction, one can optimize the combination and partitioning to achieve a desired occupancy level of beads, cells or both, within the partitions 218 that are generated.

In some cases, lysis agents, e.g., cell lysis enzymes, may be introduced into the partition with the bead stream, e.g., flowing through channel segment 204, such that lysis of the cell only commences at or after the time of partitioning. Additional reagents may also be added to the partition in this configuration, such as endonucleases to fragment the cell's DNA, DNA polymerase enzyme and dNTPs used to amplify the cell's nucleic acid fragments and to attach the barcode oligonucleotides to the amplified fragments. As noted above, in many cases, a chemical stimulus, such as DTT, may be used to release the barcodes from their respective beads into the partition. In such cases, it may be particularly desirable to provide the chemical stimulus along with the cell-containing stream in channel segment 202, such that release of the barcodes only occurs after the two streams have been combined, e.g., within the partitions 218. Where the cells are encapsulated, however, introduction of a common chemical stimulus, e.g., that both releases the oligonucleotides form their beads, and releases cells from their microcapsules may generally be provided from a separate additional side channel (not shown) upstream of or connected to channel junction 212.

As will be appreciated, a number of other reagents may be co-partitioned along with the cells, beads, lysis agents and chemical stimuli, including, for example, protective reagents, like proteinase K, chelators, nucleic acid extension, replication, transcription or amplification reagents such as polymerases, reverse transcriptases, transposases which can be used for transposon based methods (e.g., Nextera), nucleoside triphosphates or NTP analogues, primer sequences and additional cofactors such as divalent metal ions used in such reactions, ligation reaction reagents, such as ligase enzymes and ligation sequences, dyes, labels, or other tagging reagents.

The channel networks, e.g., as described herein, can be fluidly coupled to appropriate fluidic components. For example, the inlet channel segments, e.g., channel segments 202, 204, 206 and 208 are fluidly coupled to appropriate sources of the materials they are to deliver to channel junction 212. For example, channel segment 202 will be fluidly coupled to a source of an aqueous suspension of cells 214 to be analyzed, while channel segment 204 would be fluidly coupled to a source of an aqueous suspension of beads 216. Channel segments 206 and 208 would then be fluidly connected to one or more sources of the non-aqueous fluid. These sources may include any of a variety of different fluidic components, from simple reservoirs defined in or connected to a body structure of a microfluidic device, to fluid conduits that deliver fluids from off-device sources, manifolds, or the like. Likewise, the outlet channel segment 210 may be fluidly coupled to a receiving vessel or conduit for the partitioned cells. Again, this may be a reservoir defined in the body of a microfluidic device, or it may be a fluidic conduit for delivering the partitioned cells to a subsequent process operation, instrument or component.

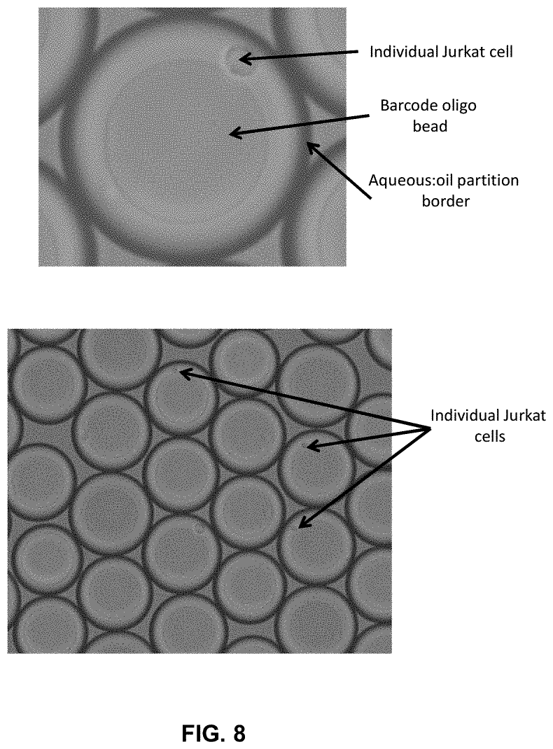

FIG. 8 shows images of individual Jurkat cells co-partitioned along with barcode oligonucleotide containing beads in aqueous droplets in an aqueous in oil emulsion. As illustrated, individual cells may be readily co-partitioned with individual beads. As will be appreciated, optimization of individual cell loading may be carried out by a number of methods, including by providing dilutions of cell populations into the microfluidic system in order to achieve the desired cell loading per partition as described elsewhere herein.

In operation, once lysed, the nucleic acid contents of the individual cells are then available for further processing within the partitions, including, e.g., fragmentation, amplification and barcoding, as well as attachment of other functional sequences. As noted above, fragmentation may be accomplished through the co-partitioning of shearing enzymes, such as endonucleases, in order to fragment the nucleic acids into smaller fragments. These endonucleases may include restriction endonucleases, including type II and type IIs restriction endonucleases as well as other nucleic acid cleaving enzymes, such as nicking endonucleases, and the like. In some cases, fragmentation may not be desired, and full length nucleic acids may be retained within the partitions, or in the case of encapsulated cells or cell contents, fragmentation may be carried out prior to partitioning, e.g., through enzymatic methods, e.g., those described herein, or through mechanical methods, e.g., mechanical, acoustic or other shearing.

Once co-partitioned, and the cells are lysed to release their nucleic acids, the oligonucleotides disposed upon the bead may be used to barcode and amplify fragments of those nucleic acids. A particularly elegant process for use of these barcode oligonucleotides in amplifying and barcoding fragments of sample nucleic acids is described in detail in U.S. Patent Application Publication No. 20140378345. Briefly, in one aspect, the oligonucleotides present on the beads that are co-partitioned with the cells, are released from their beads into the partition with the cell's nucleic acids. The oligonucleotides can include, along with the barcode sequence, a primer sequence at its 5′end. This primer sequence may be a random oligonucleotide sequence intended to randomly prime numerous different regions on the cell's nucleic acids, or it may be a specific primer sequence targeted to prime upstream of a specific targeted region of the cell's genome.

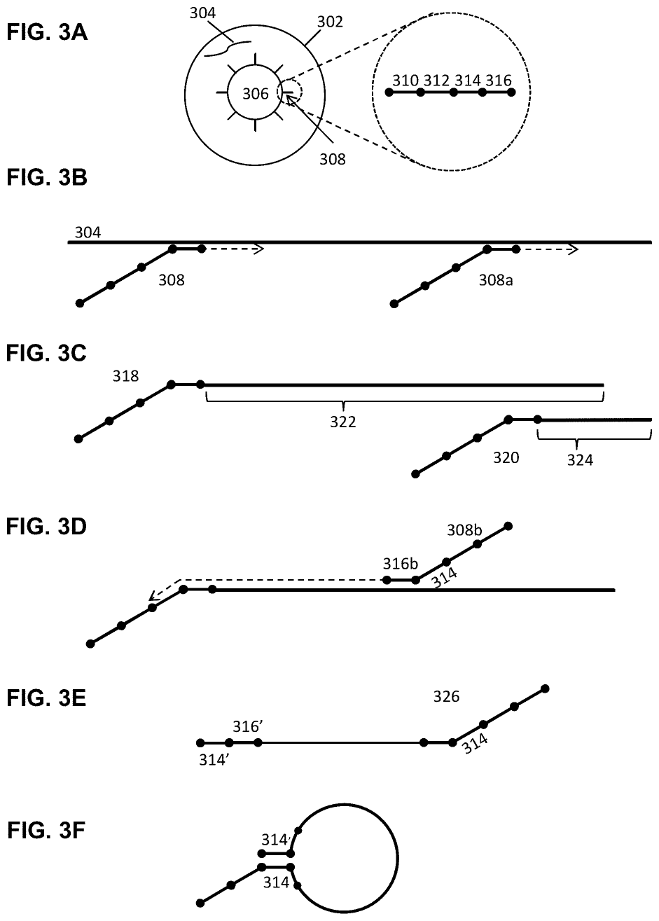

Once released, the primer portion of the oligonucleotide can anneal to a complementary region of the cell's nucleic acid. Extension reaction reagents, e.g., DNA polymerase, nucleoside triphosphates, co-factors (e.g., Mg2+ or Mn2+), that are also co-partitioned with the cells and beads, then extend the primer sequence using the cell's nucleic acid as a template, to produce a complementary fragment to the strand of the cell's nucleic acid to which the primer annealed, which complementary fragment includes the oligonucleotide and its associated barcode sequence. Annealing and extension of multiple primers to different portions of the cell's nucleic acids will result in a large pool of overlapping complementary fragments of the nucleic acid, each possessing its own barcode sequence indicative of the partition in which it was created. In some cases, these complementary fragments may themselves be used as a template primed by the oligonucleotides present in the partition to produce a complement of the complement that again, includes the barcode sequence. In some cases, this replication process is configured such that when the first complement is duplicated, it produces two complementary sequences at or near its termini, to allow formation of a hairpin structure or partial hairpin structure, the reduces the ability of the molecule to be the basis for producing further iterative copies. As described herein, the cell's nucleic acids may include any desired nucleic acids within the cell including, for example, the cell's DNA, e.g., genomic DNA, RNA, e.g., messenger RNA, and the like. For example, in some cases, the methods and systems described herein are used in characterizing expressed mRNA, including, e.g., the presence and quantification of such mRNA, and may include RNA sequencing processes as the characterization process. Alternatively or additionally, the reagents partitioned along with the cells may include reagents for the conversion of mRNA into cDNA, e.g., reverse transcriptase enzymes and reagents, to facilitate sequencing processes where DNA sequencing is employed. In some cases, where the nucleic acids to be characterized comprise RNA, e.g., mRNA, schematic illustration of one example of this is shown in FIG. 3.

As shown, oligonucleotides that include a barcode sequence are co-partitioned in, e.g., a droplet 302 in an emulsion, along with a sample nucleic acid 304. As noted elsewhere herein, the oligonucleotides 308 may be provided on a bead 306 that is co-partitioned with the sample nucleic acid 304, which oligonucleotides are releasable from the bead 306, as shown in panel A. The oligonucleotides 308 include a barcode sequence 312, in addition to one or more functional sequences, e.g., sequences 310, 314 and 316. For example, oligonucleotide 308 is shown as comprising barcode sequence 312, as well as sequence 310 that may function as an attachment or immobilization sequence for a given sequencing system, e.g., a P5 sequence used for attachment in flow cells of an Illumina Hiseq® or Miseq® system. As shown, the oligonucleotides also include a primer sequence 316, which may include a random or targeted N-mer for priming replication of portions of the sample nucleic acid 304. Also included within oligonucleotide 308 is a sequence 314 which may provide a sequencing priming region, such as a “read1” or R1 priming region, that is used to prime polymerase mediated, template directed sequencing by synthesis reactions in sequencing systems. As will be appreciated, the functional sequences may be selected to be compatible with a variety of different sequencing systems, e.g., 454 Sequencing, Ion Torrent Proton or PGM, Illumina X10, etc., and the requirements thereof. In many cases, the barcode sequence 312, immobilization sequence 310 and R1 sequence 314 may be common to all of the oligonucleotides attached to a given bead. The primer sequence 316 may vary for random N-mer primers, or may be common to the oligonucleotides on a given bead for certain targeted applications.

As will be appreciated, in some cases, the functional sequences may include primer sequences useful for RNA-seq applications. For example, in some cases, the oligonucleotides may include poly-T primers for priming reverse transcription of RNA for RNA-seq. In still other cases, oligonucleotides in a given partition, e.g., included on an individual bead, may include multiple types of primer sequences in addition to the common barcode sequences, such as both DNA-sequencing and RNA sequencing primers, e.g., poly-T primer sequences included within the oligonucleotides coupled to the bead. In such cases, a single partitioned cell may be both subjected to DNA and RNA sequencing processes.

Based upon the presence of primer sequence 316, the oligonucleotides can prime the sample nucleic acid as shown in panel B, which allows for extension of the oligonucleotides 308 and 308a using polymerase enzymes and other extension reagents also co-partitioned with the bead 306 and sample nucleic acid 304. As shown in panel C, following extension of the oligonucleotides that, for random N-mer primers, would anneal to multiple different regions of the sample nucleic acid 304; multiple overlapping complements or fragments of the nucleic acid are created, e.g., fragments 318 and 320. Although including sequence portions that are complementary to portions of sample nucleic acid, e.g., sequences 322 and 324, these constructs are generally referred to herein as comprising fragments of the sample nucleic acid 304, having the attached barcode sequences.

The barcoded nucleic acid fragments may then be subjected to characterization, e.g., through sequence analysis, or they may be further amplified in the process, as shown in panel D. For example, additional oligonucleotides, e.g., oligonucleotide 308b, also released from bead 306, may prime the fragments 318 and 320. This shown in for fragment 318. In particular, again, based upon the presence of the random N-mer primer 316b in oligonucleotide 308b (which in many cases can be different from other random N-mers in a given partition, e.g., primer sequence 316), the oligonucleotide anneals with the fragment 318, and is extended to create a complement 326 to at least a portion of fragment 318 which includes sequence 328, that comprises a duplicate of a portion of the sample nucleic acid sequence. Extension of the oligonucleotide 308b continues until it has replicated through the oligonucleotide portion 308 of fragment 318. As noted elsewhere herein, and as illustrated in panel D, the oligonucleotides may be configured to prompt a stop in the replication by the polymerase at a desired point, e.g., after replicating through sequences 316 and 314 of oligonucleotide 308 that is included within fragment 318. As described herein, this may be accomplished by different methods, including, for example, the incorporation of different nucleotides and/or nucleotide analogues that are not capable of being processed by the polymerase enzyme used. For example, this may include the inclusion of uracil containing nucleotides within the sequence region 312 to prevent a non-uracil tolerant polymerase to cease replication of that region. As a result a fragment 326 is created that includes the full-length oligonucleotide 308b at one end, including the barcode sequence 312, the attachment sequence 310, the R1 primer region 314, and the random N-mer sequence 316b. At the other end of the sequence may be included the complement 316′ to the random N-mer of the first oligonucleotide 308, as well as a complement to all or a portion of the R1 sequence, shown as sequence 314′. The R1 sequence 314 and its complement 314′ are then able to hybridize together to form a partial hairpin structure 328. As will be appreciated because the random N-mers differ among different oligonucleotides, these sequences and their complements would not be expected to participate in hairpin formation, e.g., sequence 316′, which is the complement to random N-mer 316, would not be expected to be complementary to random N-mer sequence 316b. This would not be the case for other applications, e.g., targeted primers, where the N-mers would be common among oligonucleotides within a given partition.

By forming these partial hairpin structures, it allows for the removal of first level duplicates of the sample sequence from further replication, e.g., preventing iterative copying of copies. The partial hairpin structure also provides a useful structure for subsequent processing of the created fragments, e.g., fragment 326.

In general, the amplification of the cell's nucleic acids is carried out until the barcoded overlapping fragments within the partition constitute at least 1× coverage of the particular portion or all of the cell's genome, at least 2×, at least 3×, at least 4×, at least 5×, at least 10×, at least 20×, at least 40× or more coverage of the genome or its relevant portion of interest. Once the barcoded fragments are produced, they may be directly sequenced on an appropriate sequencing system, e.g., an Illumina Hiseq®, Miseq® or X10 system, or they may be subjected to additional processing, such as further amplification, attachment of other functional sequences, e.g., second sequencing primers, for reverse reads, sample index sequences, and the like.

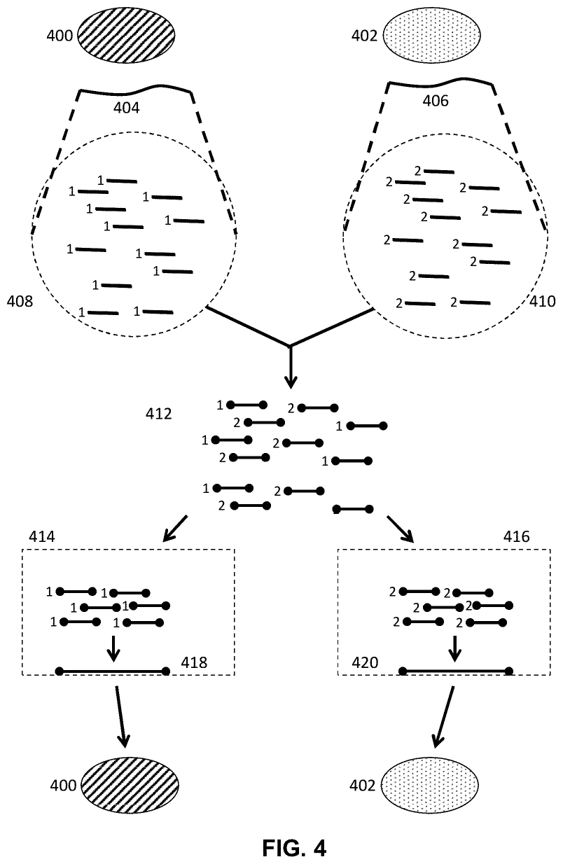

All of the fragments from multiple different partitions may then be pooled for sequencing on high throughput sequencers as described herein, where the pooled fragments comprise a large number of fragments derived from the nucleic acids of different cells or small cell populations, but where the fragments from the nucleic acids of a given cell will share the same barcode sequence. In particular, because each fragment is coded as to its partition of origin, and consequently its single cell or small population of cells, the sequence of that fragment may be attributed back to that cell or those cells based upon the presence of the barcode, which will also aid in applying the various sequence fragments from multiple partitions to assembly of individual genomes for different cells. This is schematically illustrated in FIG. 4. As shown in one example, a first nucleic acid 404 from a first cell 400, and a second nucleic acid 406 from a second cell 402 are each partitioned along with their own sets of barcode oligonucleotides as described above. The nucleic acids may comprise a chromosome, entire genome or other large nucleic acid from the cells.

Within each partition, each cell's nucleic acids 404 and 406 is then processed to separately provide overlapping set of second fragments of the first fragment(s), e.g., second fragment sets 408 and 410. This processing also provides the second fragments with a barcode sequence that is the same for each of the second fragments derived from a particular first fragment. As shown, the barcode sequence for second fragment set 408 is denoted by “1” while the barcode sequence for fragment set 410 is denoted by “2”. A diverse library of barcodes may be used to differentially barcode large numbers of different fragment sets. However, it is not necessary for every second fragment set from a different first fragment to be barcoded with different barcode sequences. In fact, in many cases, multiple different first fragments may be processed concurrently to include the same barcode sequence. Diverse barcode libraries are described in detail elsewhere herein.

The barcoded fragments, e.g., from fragment sets 408 and 410, may then be pooled for sequencing using, for example, sequence by synthesis technologies available from Illumina or Ion Torrent division of Thermo-Fisher, Inc. Once sequenced, the sequence reads 412 can be attributed to their respective fragment set, e.g., as shown in aggregated reads 414 and 416, at least in part based upon the included barcodes, and in some cases, in part based upon the sequence of the fragment itself. The attributed sequence reads for each fragment set are then assembled to provide the assembled sequence for each cell's nucleic acids, e.g., sequences 418 and 420, which in turn, may be attributed to individual cells, e.g., cells 400 and 402.

While described in terms of analyzing the genetic material present within cells, the methods and systems described herein may have much broader applicability, including the ability to characterize other aspects of individual cells or cell populations, by allowing for the allocation of reagents to individual cells, and providing for the attributable analysis or characterization of those cells in response to those reagents. These methods and systems are particularly valuable in being able to characterize cells for, e.g., research, diagnostic, pathogen identification, and many other purposes. By way of example, a wide range of different cell surface features, e.g., cell surface proteins like cluster of differentiation or CD proteins, have significant diagnostic relevance in characterization of diseases like cancer.

In one particularly useful application, the methods and systems described herein may be used to characterize cell features, such as cell surface features, e.g., proteins, receptors, etc. In particular, the methods described herein may be used to attach reporter molecules to these cell features, that when partitioned as described above, may be barcoded and analyzed, e.g., using DNA sequencing technologies, to ascertain the presence, and in some cases, relative abundance or quantity of such cell features within an individual cell or population of cells.

In a particular example, a library of potential cell binding ligands, e.g., antibodies, antibody fragments, cell surface receptor binding molecules, or the like, maybe provided associated with a first set of nucleic acid reporter molecules, e.g., where a different reporter oligonucleotide sequence is associated with a specific ligand, and therefore capable of binding to a specific cell surface feature. In some aspects, different members of the library may be characterized by the presence of a different oligonucleotide sequence label, e.g., an antibody to a first type of cell surface protein or receptor would have associated with it a first known reporter oligonucleotide sequence, while an antibody to a second receptor protein would have a different known reporter oligonucleotide sequence associated with it. Prior to co-partitioning, the cells would be incubated with the library of ligands, that may represent antibodies to a broad panel of different cell surface features, e.g., receptors, proteins, etc., and which include their associated reporter oligonucleotides. Unbound ligands are washed from the cells, and the cells are then co-partitioned along with the barcode oligonucleotides described above. As a result, the partitions will include the cell or cells, as well as the bound ligands and their known, associated reporter oligonucleotides.

Without the need for lysing the cells within the partitions, one could then subject the reporter oligonucleotides to the barcoding operations described above for cellular nucleic acids, to produce barcoded, reporter oligonucleotides, where the presence of the reporter oligonucleotides can be indicative of the presence of the particular cell surface feature, and the barcode sequence will allow the attribution of the range of different cell surface features to a given individual cell or population of cells based upon the barcode sequence that was co-partitioned with that cell or population of cells. As a result, one may generate a cell-by-cell profile of the cell surface features within a broader population of cells. This aspect of the methods and systems described herein, is described in greater detail below.

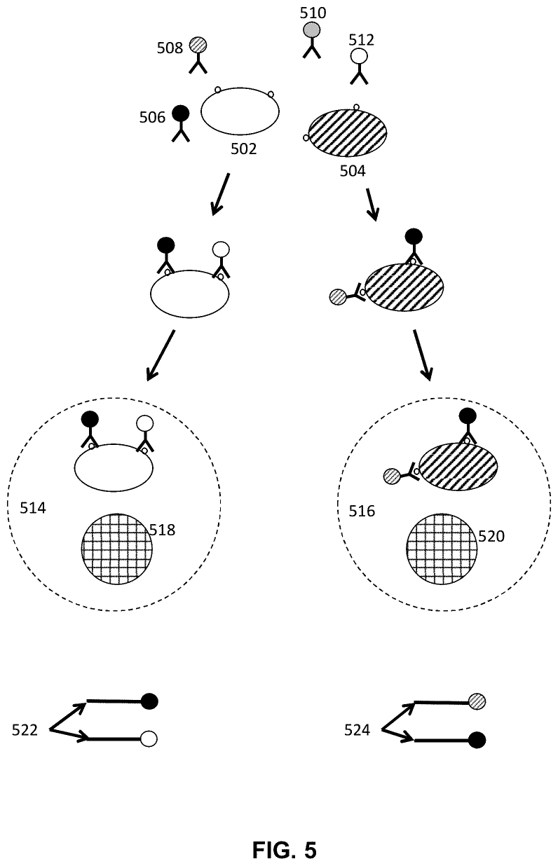

This example is schematically illustrated in FIG. 5. As shown, a population of cells, represented by cells 502 and 504 are incubated with a library of cell surface associated reagents, e.g., antibodies, cell surface binding proteins, ligands or the like, where each different type of binding group includes an associated nucleic acid reporter molecule associated with it, shown as ligands and associated reporter molecules 506, 508, 510 and 512 (with the reporter molecules being indicated by the differently shaded circles). Where the cell expresses the surface features that are bound by the library, the ligands and their associated reporter molecules can become associated or coupled with the cell surface. Individual cells are then partitioned into separate partitions, e.g., droplets 514 and 516, along with their associated ligand/reporter molecules, as well as an individual barcode oligonucleotide bead as described elsewhere herein, e.g., beads 522 and 524, respectively. As with other examples described herein, the barcoded oligonucleotides are released from the beads and used to attach the barcode sequence the reporter molecules present within each partition with a barcode that is common to a given partition, but which varies widely among different partitions. For example, as shown in FIG. 5, the reporter molecules that associate with cell 502 in partition 514 are barcoded with barcode sequence 518, while the reporter molecules associated with cell 504 in partition 516 are barcoded with barcode 520. As a result, one is provided with a library of oligonucleotides that reflects the surface ligands of the cell, as reflected by the reporter molecule, but which is substantially attributable to an individual cell by virtue of a common barcode sequence, allowing a single cell level profiling of the surface characteristics of the cell. As will be appreciated, this process is not limited to cell surface receptors but may be used to identify the presence of a wide variety of specific cell structures, chemistries or other characteristics.

V. Detection of Subpopulations of Cells within a Heterogeneous Cell Population

The single cell processing and analysis methods and systems described herein can be utilized for various applications, including analysis of specific individual cells, analysis of different cell types within populations of differing cell types, analysis and characterization of large populations of cells for environmental, human health, epidemiological, forensic, or any of a wide variety of different applications. Sequence variation in transcriptome data obtained from a cell population using the systems and methods disclosed herein can be used to identify distinct subpopulations of cells with a heterogeneous cell sample.

In an aspect, the present disclosure provides a method of distinguishing a minor cell population from a major cell population in a heterogeneous cell sample. The method comprises: (a) partitioning a plurality of cells of a heterogeneous cell sample into a plurality of droplets, wherein upon partitioning, a given droplet of the plurality of droplets comprises a given cell of the plurality of cells and a given bead of a plurality of beads comprising a plurality of oligonucleotide barcodes, wherein the given cell comprises a first set of polynucleotides; (b) subjecting the first set of polynucleotides to nucleic acid amplification under conditions sufficient to generate a second set of polynucleotides, wherein a given polynucleotide of the second set of polynucleotides comprises (i) a segment having a sequence of a polynucleotide of the first set or a complement thereof and (ii) a segment having a sequence of a oligonucleotide barcode of the plurality of oligonucleotide barcodes or a complement thereof; (c) generating a library of polynucleotides from a pool of polynucleotides comprising a plurality of second sets of polynucleotides, including the second set of polynucleotides, from the plurality of droplets; (d) subjecting the library of polynucleotides to sequencing to yield sequencing reads, wherein barcode sequences of the plurality of oligonucleotide barcodes associate sequencing reads with individual cells of the plurality of cells of the heterogeneous cell sample; and (e) processing the sequencing reads associated with individual cells of the plurality of cells of the heterogeneous cell sample to generate (i) a first set of genetic aberrations corresponding to the minor cell population and (ii) a second set of genetic aberrations corresponding to the major cell population, which first and second set of genetic aberrations differentiate a cell of the minor cell population from a cell of the major cell population. The method, in some cases, further comprises releasing the first set of polynucleotides from the given cell into the given droplet subsequent to (a). In some embodiments, nucleic acid amplification reagents are co-partitioned in the given droplet. Such reagents include, but are not limited to, enzymes such as polymerases and reverse transcriptases, primers and oligonucleotides such as amplification primers and template switching oligonucleotides, dNTPs, co-factors, etc.

In some embodiments, the given bead of the given droplet is a gel bead. The given bead of the given droplet can comprise at least 1,000,000 oligonucleotide barcodes. In some embodiments, each oligonucleotide barcode of the given bead of the given droplet comprises a barcode sequence identical to all other oligonucleotide barcodes of the given bead of the given droplet and a molecular identifier sequence (e.g., a unique molecular identifier, UMI) not identical to all other oligonucleotide barcodes of the given bead of the given droplet. The barcode sequence of an oligonucleotide barcode, as previously described, can be used for later attribution of, e.g., sequence information, to a particular cell. In addition to a barcode sequence and a molecular identifier sequence, the oligonucleotide barcodes can further comprise primer binding sequences (e.g., amplification, sequencing, etc), sample index sequences, regions which function as a primer for base extension reactions, and other sequences for downstream sample processing. In some embodiments, the method further comprises applying a stimulus to the given droplet to release the oligonucleotide barcodes from the given bead into the given droplet. This stimulus can be, for example, a chemical stimulus, optical stimulus such as light, or thermal stimulus such as an increase in temperature.

Where desired, the method further comprises determining a percentage of the heterogeneous cell sample represented by the minor cell population and/or the major cell population. The percentage of the heterogeneous cell sample represented by the minor cell population can be determined at a sensitivity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The percentage of the heterogeneous cell sample represented by the major cell population can be determined at a sensitivity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

A heterogeneous cell sample can comprise at least two cell types, and in some cases more than two types (e.g., at least 3, 4, 5, 6, 7, 8, 9, 10 or more). In cases where the heterogeneous cell sample comprises greater than two types of cells, the minor cell population can refer to the population to be analyzed and the major cell population comprises the remainder of the cells in the heterogeneous cell population. In various embodiments, the minor cell population represents at least about 1% of the heterogeneous cell sample. In some cases, the minor cell population represents about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49% of the heterogeneous cell sample. In some cases, the minor cell population represents at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49% of the heterogeneous cell sample. In various embodiments, the minor cell population represents less than about 50% of the heterogeneous cell sample. The major cell population, in some cases, represents greater than about 50% of the heterogeneous cell sample. In some cases, the major cell population represents about 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the heterogeneous cell sample. The major cell population, in some cases, represents less than about 100% of the heterogeneous cell sample.

In some embodiments, the heterogeneous cell sample comprises cells obtained from a biological sample. In some cases, the biological sample comprises bone marrow or any portion or derivative thereof. The bone marrow can be obtained from a subject undergoing or having undergone a bone marrow transplant. In some cases, the heterogeneous cell sample comprises cells that have been cryopreserved.

In some embodiments, the first set of genetic aberrations and the second set of genetic aberrations are associated or suspected of being (individually) associated with a minor cell population and a major cell population, that is the first set of genetic aberrations is suspected of being uniquely associated with a minor cell population and the second set of genetic aberrations is suspected of being uniquely associated with a major cell population. The first and second sets of genetic aberrations can be used to differentiate a cell of the minor cell population from a cell of the major cell population. Examples of genetic aberrations include, but are not limited to, polymorphisms such as single nucleotide variations (SNVs), insertions, deletions, repeats, small insertions, small deletions, small repeats, structural variant junctions, variable length tandem repeats, and/or flanking sequences. In some embodiments, the first and second sets of genetic aberrations comprise a single type of aberration. The first and second sets of genetic aberrations can comprise single nucleotide variants (SNVs). Each of the first and second set of genetic aberrations can comprise at least 30, 40, 50, 60, 70, 80, 90, 100, 110, 120, 130, 140, 150, 200, 250, 500, 750, 1,000 SNVs or more. In various embodiments, the first set of genetic aberrations and the second set of genetic aberrations do not intersect (e.g., do not share members). In some embodiments, the first and second sets of genetic aberrations comprise multiple types of aberrations.

In an aspect, the disclosure provides a method of distinguishing a first cell population from a second cell population in a heterogeneous cell sample. The method comprises: (a) partitioning a plurality of cells of a heterogeneous cell sample into a plurality of droplets, wherein upon partitioning, a given droplet of the plurality of droplets comprises a given cell of the plurality of cells and a given bead of a plurality of beads comprising a plurality of oligonucleotide barcodes, wherein the given cell comprises a first set of polynucleotides; (b) subjecting the first set of polynucleotides to nucleic acid amplification under conditions sufficient to generate a second set of polynucleotides, wherein a given polynucleotide of the second set of polynucleotides comprises (i) a segment having a sequence of a polynucleotide of the first set or a complement thereof and (ii) a segment having a sequence of a oligonucleotide barcode of the plurality of oligonucleotide barcodes or a complement thereof; (c) generating a library of polynucleotides from a pool of polynucleotides comprising a plurality of second sets of polynucleotides, including the second set of polynucleotides, from the plurality of droplets; (d) subjecting the library of polynucleotides to sequencing to yield sequencing reads, wherein barcode sequences of the plurality of oligonucleotide barcodes associate sequencing reads with individual cells of the plurality of cells of the heterogeneous cell sample; and (e) determining a percentage of the heterogeneous cell sample represented by the first cell population using a first set of genetic aberrations corresponding to the first cell population and a second set of genetic aberrations corresponding to the second cell population obtained from processing the sequencing reads associated with individual cells of the heterogeneous cell sample. In some embodiments, the percentage of the heterogeneous cell sample represented by the first cell population can be determined at a sensitivity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 96%, 98%, or 99%. In some embodiments, the method further comprises determining a percentage of the heterogeneous cell population represented by the second cell population. The percentage of the heterogeneous cell sample represented by the second cell population can be determined at a sensitivity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 96%, 98%, or 99%.

The method, in some cases, further comprises releasing the first set of polynucleotides from the given cell into the given droplet subsequent to (a). In some embodiments, nucleic acid amplification reagents are co-partitioned in the given droplet. Such reagents include, but are not limited to, enzymes such as polymerases and reverse transcriptases, primers and oligonucleotides such as amplification primers and template switching oligonucleotides, dNTPs, co-factors, etc.

In some embodiments, the given bead of the given droplet is a gel bead. The given bead of the given droplet can comprise at least 1,000,000 oligonucleotide barcodes. In some embodiments, each oligonucleotide barcode of the given bead of the given droplet comprises a barcode sequence identical to all other oligonucleotide barcodes of the given bead of the given droplet and a molecular identifier sequence (e.g., a unique molecular identifier, UMI) not identical to all other oligonucleotide barcodes of the given bead of the given droplet. The barcode sequence of an oligonucleotide barcode, as previously described, can be used for later attribution of, e.g., sequence information, to a particular cell. In addition to a barcode sequence and a molecular identifier sequence, the oligonucleotide barcodes can further comprise primer binding sequences (e.g., amplification, sequencing, etc), sample index sequences, regions which function as a primer for base extension reactions, and other sequences for downstream sample processing. In some embodiments, the method further comprises applying a stimulus to the given droplet to release the oligonucleotide barcodes from the given bead into the given droplet. This stimulus can be, for example, a chemical stimulus, optical stimulus such as light, or thermal stimulus such as an increase in temperature.

A heterogeneous cell sample can comprise at least two cells, and in some cases more than two types (e.g., at least 3, 4, 5, 6, 7, 8, 9, 10 or more). In cases where the heterogeneous cell sample comprises more than two types of cells, the first cell population can refer to the population to be analyzed and the second cell population comprises the remainder of the cells in the heterogeneous cell sample. In various embodiments, the first cell population represents at least about 1% of the heterogeneous cell sample. In some cases, the first cell population represents about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49% of the heterogeneous cell sample. In some cases, the first cell population represents at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 49% of the heterogeneous cell sample. In various embodiments, the first cell population represents less than about 50% of the heterogeneous cell sample. The second cell population, in some cases, represents greater than about 50% of the heterogeneous cell sample. In some cases, the second cell population represents about 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the heterogeneous cell sample. The second cell population, in some cases, represents less than about 100% of the heterogeneous cell sample.

In some embodiments, the heterogeneous cell sample comprises cells obtained from a biological sample. In some cases, the biological sample comprises bone marrow or any portion or derivative thereof. The bone marrow can be obtained from a subject undergoing or having undergone a bone marrow transplant. In some cases, the heterogeneous cell sample comprises cells that have been cryopreserved.

In some embodiments, the first set of genetic aberrations and the second set of genetic aberrations are associated or suspected of being (individually) associated with a first cell population and a second cell population, that is the first set of genetic aberrations is suspected of being uniquely associated with a first cell population and the second set of genetic aberrations is suspected of being uniquely associated with a second cell population. The first and second sets of genetic aberrations can be used to differentiate a cell of the first cell population from a cell of the second cell population. Examples of genetic aberrations include, but are not limited to, polymorphisms such as single nucleotide variations (SNVs), insertions, deletions, repeats, small insertions, small deletions, small repeats, structural variant junctions, variable length tandem repeats, and/or flanking sequences. In some embodiments, the first and second sets of genetic aberrations comprise a single type of aberration. The first and second sets of genetic aberrations can comprise single nucleotide variants (SNVs). Each of the first and second set of genetic aberrations can comprise at least 30, 40, 50, 60, 70, 80, 90, 100, 110, 120, 130, 140, 150, 200, 250, 500, 750, 1,000 SNVs or more. In various embodiments, the first set of genetic aberrations and the second set of genetic aberrations do not intersect (e.g., do not share members). In some embodiments, the first and second sets of genetic aberrations comprise multiple types of aberrations.

In an aspect, the disclosure provides a method of determining a percentage of a cell population in a heterogeneous cell sample at a sensitivity of at least about 95%, wherein the cell population represents less than about 10% of the heterogeneous cell sample, comprising: (a) partitioning a plurality of cells of a heterogeneous cell sample into a plurality of droplets, wherein upon partitioning, a given droplet of the plurality of droplets comprises a given cell of the plurality of cells and a given bead of a plurality of beads comprising a plurality of oligonucleotide barcodes, wherein the given cell comprises a first set of polynucleotides; (b) subjecting the first set of polynucleotides to nucleic acid amplification under conditions sufficient to generate a second set of polynucleotides, wherein a given polynucleotide of the second set of polynucleotides comprises (i) a segment having a sequence of a polynucleotide of the first set or a complement thereof and (ii) a segment having a sequence of a oligonucleotide barcode or a complement thereof; (c) generating a library of polynucleotides from a pool of polynucleotides comprising a plurality of second sets of polynucleotides, including the second set of polynucleotides, from the plurality of droplets; (d) subjecting the library of polynucleotides to sequencing to yield sequencing reads, wherein barcode sequences of the plurality oligonucleotide barcodes associate sequencing reads with individual cells of the plurality of cells of the heterogeneous cell sample; (e) determining, with a sensitivity of at least about 95%, a percentage of the heterogeneous cell sample represented by the cell population using a first set of genetic aberrations and a second set of genetic aberrations obtained from processing the sequencing reads associated with individual cells of the heterogeneous cell sample, wherein the cell population represents less than about 10% of the heterogeneous cell sample.

The method, in some cases, further comprises releasing the first set of polynucleotides from the given cell into the given droplet subsequent to (a). In some embodiments, nucleic acid amplification reagents are co-partitioned in the given droplet. Such reagents include, but are not limited to, enzymes such as polymerases and reverse transcriptases, primers and oligonucleotides such as amplification primers and template switching oligonucleotides, dNTPs, co-factors, etc.

In various embodiments of the aspects described herein, the given bead of the given droplet is a gel bead. The given bead of the given droplet can comprise at least 1,000,000 oligonucleotide barcodes. In some embodiments, each oligonucleotide barcode of the given bead of the given droplet comprises a barcode sequence identical to all other oligonucleotide barcodes of the given bead of the given droplet and a molecular identifier sequence (e.g., a unique molecular identifier, UMI) not identical to all other oligonucleotide barcodes of the given bead of the given droplet. The barcode sequence of an oligonucleotide barcode, as previously described, can be used for later attribution of, e.g., sequence information, to a particular cell. In addition to a barcode sequence and a molecular identifier sequence, the oligonucleotide barcodes can further comprise primer binding sequences (e.g., amplification, sequencing, etc), sample index sequences, regions which function as a primer for base extension reactions, and other sequences for downstream sample processing. In some embodiments, the method further comprises applying a stimulus to the given droplet to release the oligonucleotide barcodes from the given bead into the given droplet. This stimulus can be, for example, a chemical stimulus, optical stimulus such as light, or thermal stimulus such as an increase in temperature.

A heterogeneous cell sample can comprise at least two cells, and in some cases more than two types (e.g., at least 3, 4, 5, 6, 7, 8, 9, 10 or more). In cases where the heterogeneous cell sample comprises more than two types of cells, the cell population to be analyzed represents a percentage of the total heterogeneous cell population. In various embodiments, the cell population to be analyzed represents at least about 1% of the heterogeneous cell sample. In some cases, the cell population represents about 1%, 2%, 3%, 4%, 5%, 6%, 7%, 8%, 9%, or 10%. The percentage of the heterogeneous cell sample represented by the cell population can be determined at a sensitivity of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 96%, 98%, or 99%.

In some embodiments, the heterogeneous cell sample comprises cells obtained from a biological sample. In some cases, the biological sample comprises bone marrow or any portion or derivative thereof. The bone marrow can be obtained from a subject undergoing or having undergone a bone marrow transplant. In some cases, the heterogeneous cell sample comprises cells that have been cryopreserved.

In some embodiments, one of the first set of genetic aberrations and the second set of genetic aberrations is associated or suspected of being associated with the cell population to be analyzed. The first and second sets of genetic aberrations can be used to differentiate a cell of the cell population from other cell types of heterogeneous cell sample. Examples of genetic aberrations include, but are not limited to, polymorphisms such as single nucleotide variations (SNVs), insertions, deletions, repeats, small insertions, small deletions, small repeats, structural variant junctions, variable length tandem repeats, and/or flanking sequences. In some embodiments, the first and second sets of genetic aberrations comprise a single type of aberration. The first and second sets of genetic aberrations can comprise single nucleotide variants (SNVs). Each of the first and second set of genetic aberrations can comprise at least 30, 40, 50, 60, 70, 80, 90, 100, 110, 120, 130, 140, 150, 200, 250, 500, 750, 1,000 SNVs or more. In various embodiments, the first set of genetic aberrations and the second set of genetic aberrations do not intersect (e.g., do not share members). In some embodiments, the first and second sets of genetic aberrations comprise multiple types of aberrations.

In various embodiments of the aspects disclosed herein, a heterogeneous cell sample can be obtained from any of various sources. A heterogeneous cell sample may be directly obtained from or derived from blood and other liquid samples of biological origin, solid tissue samples such as a biopsy specimen or tissue cultures or cells derived therefrom, and the progeny thereof. A heterogeneous cell sample can include those which have been manipulated in any way after their procurement, such as by treatment with reagents, solubilization, or enrichment for certain components, such as proteins or polynucleotides, or embedding in a semi-solid or solid matrix for sectioning purposes. Biological sample includes clinical samples, such as cells in culture, cell supernatants, cell lysates, serum, plasma, biological fluid, and tissue samples. The source of the biological sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. In some embodiments, the biological sample is obtained from a primary or metastatic tumor. The biological sample may contain compounds which are not naturally intermixed with the tissue in nature such as preservatives, anticoagulants, buffers, fixatives, nutrients, antibiotics, or the like. Cells can be obtained from sources such as prostate, breast, skin, muscle, facia, brain, endometrium, lung, head and neck, pancreas, small intestine, blood, liver, testes, ovaries, colon, skin, stomach, esophagus, spleen, lymph node, bone marrow, kidney, placenta, or fetus. Samples can comprise peripheral blood, lymph fluid, ascites, serous fluid, pleural effusion, sputum, bronchial wash, bronchioalveolar lavage fluid (BALF), cerebrospinal fluid, semen, amniotic fluid, lacrimal fluid, stool, or urine.

The single cell analysis processes described herein is used to characterize cancer cells. In particular, conventional analytical techniques, including the ensemble sequencing processes alluded to above, are not highly adept at picking small variations in genomic make-up of cancer cells, particularly where those exist in a sea of normal tissue cells. Further, even as between tumor cells, wide variations can exist and can be masked by the ensemble approaches to sequencing (See, e.g., Patel, et al., Single-cell RNA-seq highlights intratumoral heterogeneity in primary glioblastoma, Science DOI: 10.1126/science.1254257 (Published online Jun. 12, 2014). Cancer cells may be derived from solid tumors (e.g., via biopsies or from surgical procedures), hematological malignancies, cell lines, or obtained as circulating tumor cells, and subjected to the partitioning processes described above. Upon analysis, one can identify individual cell sequences as deriving from a single cell or small group of cells, and distinguish those over normal tissue cell sequences. Further, as described in co-pending U.S. Patent Application Publication No. 20150376700 the full disclosure of which is hereby incorporated herein by reference in its entirety for all purposes, one may also obtain phased sequence information from each cell, allowing clearer characterization of the haplotype variants within a cancer cell. The single cell analysis approach is particularly useful for systems and methods involving low quantities of input nucleic acids, as described in co-pending U.S. Patent Application Publication No. 20150376605, the full disclosure of which is hereby incorporated herein by reference in its entirety for all purposes.

As with cancer cell analysis, the analysis and diagnosis of fetal health or abnormality through the analysis of fetal cells is a difficult task using conventional techniques. In particular, in the absence of relatively invasive procedures, such as amniocentesis obtaining fetal cell samples can employ harvesting those cells from the maternal circulation (e.g., via venipuncture or blood draw). As will be appreciated, such circulating fetal cells make up an extremely small fraction of the overall cellular population of that circulation. As a result complex analyses are performed in order to characterize what of the obtained data is likely derived from fetal cells as opposed to maternal cells. By employing the single cell characterization methods and systems described herein, however, one can attribute genetic make up to individual cells, and categorize those cells as maternal or fetal based upon their respective genetic make-up. Further, the genetic sequence of fetal cells may be used to identify any of a number of genetic disorders, including, e.g., aneuploidy such as Down syndrome, Edwards syndrome, and Patau syndrome.

In some embodiments, the single cell analysis processes described herein is used to study and/or evaluate graft vs. host disease in transplantation studies, where cells from a donor are mixed with cells of a recipient. Transplant rejection can occur when transplanted tissue is rejected by the recipient's immune system, which destroys the transplanted tissue. For example, transplantation of hematopoietic stem cells (hematopoietic stem cell transplantation, HSCT), which are multipotent stem cells usually derived from bone marrow, peripheral blood, or umbilical cord blood, is often performed for patients with certain cancers of the blood or bone marrow. In these cases, the recipient's immune system is usually destroyed with radiation or chemotherapy before the transplantation so as to reduce the likelihood of rejection by the immune system. However, HSCT remains a dangerous procedure with many possible complications. The single cell analysis processes described herein can be useful in assaying bone marrow derived cells, for example, in evaluating and monitoring the coexistence of recipient's and donor's hematopoietic systems after allogeneic marrow transplantation (e.g., chimerism or mixed chimerism). Such analysis can be useful for discovering new insights into the disease state of the recipient before and after transplant that are not readily achievable with traditional PCR such as digital PCR, FACS-based analysis and other methods.

The ability to characterize individual cells from larger diverse populations of cells is also of significant value in both environmental testing as well as in forensic analysis, where samples may, by their nature, be made up of diverse populations of cells and other material that “contaminate” the sample, relative to the cells for which the sample is being tested, e.g., environmental indicator organisms, toxic organisms, and the like for, e.g., environmental and food safety testing, victim and/or perpetrator cells in forensic analysis for sexual assault, and other violent crimes, and the like.

Additional useful applications of the above described single cell sequencing and characterization processes are in the field of neuroscience research and diagnosis. In particular, neural cells can include long interspersed nuclear elements (LINEs), or ‘jumping’ genes that can move around the genome, which cause each neuron to differ from its neighbor cells. Research has shown that the number of LINEs in human brain exceeds that of other tissues, e.g., heart and liver tissue, with between 80 and 300 unique insertions (See, e.g., Coufal, N. G. et al. Nature 460, 1127-1131 (2009)). These differences have been postulated as being related to a person's susceptibility to neuro-logical disorders (see, e.g., Muotri, A. R. et al. Nature 468, 443-446 (2010)), or provide the brain with a diversity with which to respond to challenges. As such, the methods described herein may be used in the sequencing and characterization of individual neural cells.

Using the methods and systems described herein, RNA transcripts present in individual cells, populations of cells, or subsets of populations of cells can be isolated and analyzed for transcriptome analysis. In particular, in some cases, the barcode oligonucleotides may be configured to prime, replicate and consequently yield barcoded fragments of RNA from individual cells. For example, in some cases, the barcode oligonucleotides may include mRNA specific priming sequences, e.g., poly-T primer segments that allow priming and replication of mRNA in a reverse transcription reaction or other targeted priming sequences. Alternatively or additionally, random RNA priming may be carried out using random N-mer primer segments of the barcode oligonucleotides.

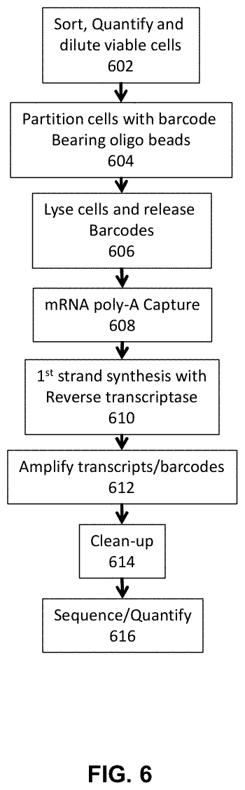

FIG. 6 provides a schematic of one example method for RNA expression analysis in individual cells using the methods described herein. As shown, at operation 602 a cell containing sample is sorted for viable cells, which are quantified and diluted for subsequent partitioning. At operation 604, the individual cells separately co-partitioned with gel beads bearing the barcoding oligonucleotides as described herein. The cells are lysed and the barcoded oligonucleotides released into the partitions at operation 606, where they interact with and hybridize to the mRNA at operation 608, e.g., by virtue of a poly-T primer sequence, which is complementary to the poly-A tail of the mRNA. Using the poly-T barcode oligonucleotide as a priming sequence, a reverse transcription reaction is carried out at operation 610 to synthesize a cDNA transcript of the mRNA that includes the barcode sequence. The barcoded cDNA transcripts are then subjected to additional amplification at operation 612, e.g., using a PCR process, purification at operation 614, before they are placed on a nucleic acid sequencing system for determination of the cDNA sequence and its associated barcode sequence(s). In some cases, as shown, operations 602 through 608 can occur while the reagents remain in their original droplet or partition, while operations 612 through 616 can occur in bulk (e.g., outside of the partition). In the case where a partition is a droplet in an emulsion, the emulsion can be broken and the contents of the droplet pooled in order to complete operations 612 through 616. In some cases, barcode oligonucleotides may be digested with exonucleases after the emulsion is broken. Exonuclease activity can be inhibited by ethylenediaminetetraacetic acid (EDTA) following primer digestion. In some cases, operation 610 may be performed either within the partitions based upon co-partitioning of the reverse transcription mixture, e.g., reverse transcriptase and associated reagents, or it may be performed in bulk.

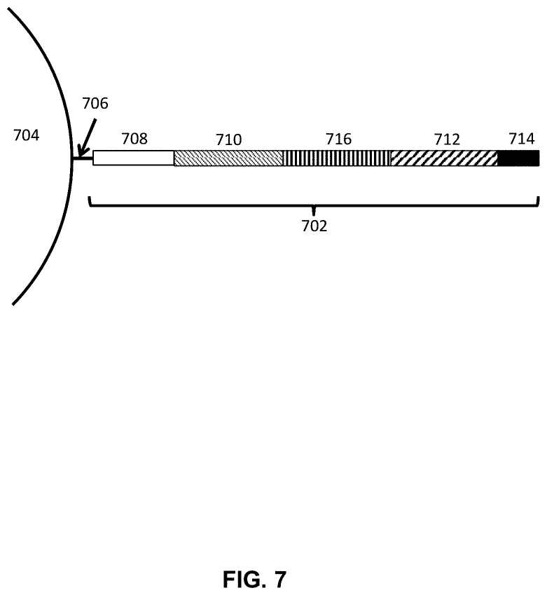

As noted elsewhere herein, the structure of the barcode oligonucleotides may include a number of sequence elements in addition to the oligonucleotide barcode sequence. One example of a barcode oligonucleotide for use in RNA analysis as described above is shown in FIG. 7. As shown, the overall oligonucleotide 702 is coupled to a bead 704 by a releasable linkage 706, such as a disulfide linker. The oligonucleotide may include functional sequences that are used in subsequent processing, such as functional sequence 708, which may include one or more of a sequencer specific flow cell attachment sequence, e.g., a P5 sequence for Illumina sequencing systems, as well as sequencing primer sequences, e.g., a R1 primer for Illumina sequencing systems. A barcode sequence 710 is included within the structure for use in barcoding the sample RNA. An mRNA specific priming sequence, such as poly-T sequence 712 is also included in the oligonucleotide structure. An anchoring sequence segment 714 may be included to ensure that the poly-T sequence hybridizes at the sequence end of the mRNA. This anchoring sequence can include a random short sequence of nucleotides, e.g., 1-mer, 2-mer, 3-mer or longer sequence, which will ensure that the poly-T segment is more likely to hybridize at the sequence end of the poly-A tail of the mRNA. An additional sequence segment 716 may be provided within the oligonucleotide sequence. In some cases, this additional sequence provides a unique molecular sequence segment, e.g., as a random sequence (e.g., such as a random N-mer sequence) that varies across individual oligonucleotides coupled to a single bead, whereas barcode sequence 710 can be constant among oligonucleotides tethered to an individual bead. This unique sequence serves to provide a unique identifier of the starting mRNA molecule that was captured, in order to allow quantitation of the number of original expressed RNA. As will be appreciated, although shown as a single oligonucleotide tethered to the surface of a bead, individual bead can include tens to hundreds of thousands or even millions of individual oligonucleotide molecules, where, as noted, the barcode segment can be constant or relatively constant for a given bead, but where the variable or unique sequence segment will vary across an individual bead. This unique molecular sequence segment may include from 5 to about 8 or more nucleotides within the sequence of the oligonucleotides. In some cases, the unique molecular sequence segment can be 2, 3, 4, 5, 6, 7, 8, 9, 10, 11, 12, 13, 14, 15, 16, 17, 18, 19 or 20 nucleotides in length or longer. In some cases, the unique molecular sequence segment can be at least 2, 3, 4, 5, 6, 7, 8, 9, 10, 11, 12, 13, 14, 15, 16, 17, 18, 19 or 20 nucleotides in length or longer. In some cases, the unique molecular sequence segment can be at most 2, 3, 4, 5, 6, 7, 8, 9, 10, 11, 12, 13, 14, 15, 16, 17, 18, 19 or 20 nucleotides in length or shorter.

In operation, and with reference to FIGS. 6 and 7, a cell is co-partitioned along with a barcode bearing bead and lysed while the barcoded oligonucleotides are released from the bead. The poly-T portion of the released barcode oligonucleotide then hybridizes to the poly-A tail of the mRNA. The poly-T segment then primes the reverse transcription of the mRNA to produce a cDNA transcript of the mRNA, but which includes each of the sequence segments 708-716 of the barcode oligonucleotide. Again, because the oligonucleotide 702 includes an anchoring sequence 714, it will more likely hybridize to and prime reverse transcription at the sequence end of the poly-A tail of the mRNA. Within any given partition, all of the cDNA transcripts of the individual mRNA molecules will include a common barcode sequence segment 710. However, by including the unique random N-mer sequence, the transcripts made from different mRNA molecules within a given partition will vary at this unique sequence. This provides a quantitation feature that can be identifiable even following any subsequent amplification of the contents of a given partition, e.g., the number of unique segments associated with a common barcode can be indicative of the quantity of mRNA originating from a single partition, and thus, a single cell. As noted above, the transcripts are then amplified, cleaned up and sequenced to identify the sequence of the cDNA transcript of the mRNA, as well as to sequence the barcode segment and the unique sequence segment.

As noted elsewhere herein, while a poly-T primer sequence is described, other targeted or random priming sequences may also be used in priming the reverse transcription reaction. Likewise, although described as releasing the barcoded oligonucleotides into the partition along with the contents of the lysed cells, it will be appreciated that in some cases, the gel bead bound oligonucleotides may be used to hybridize ad capture the mRNA on the solid phase of the gel beads, in order to facilitate the separation of the RNA from other cell contents.

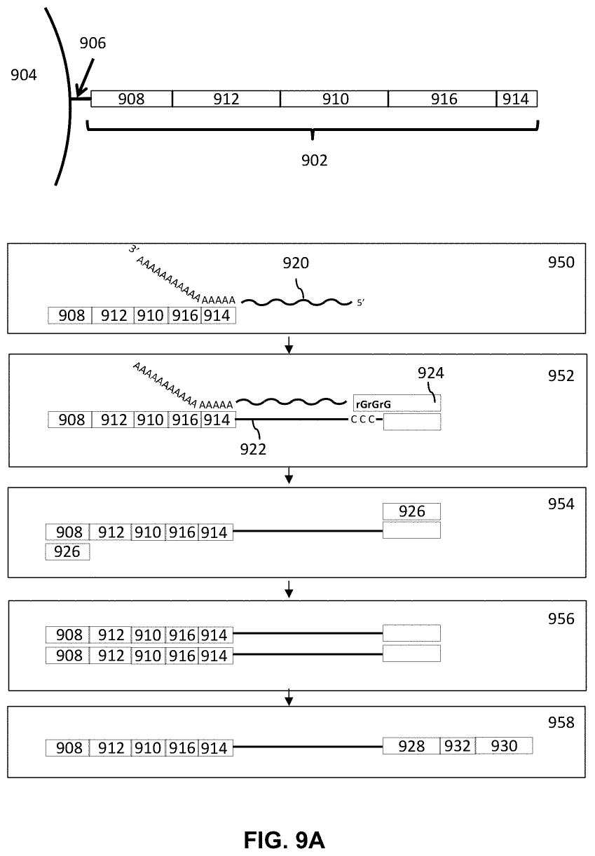

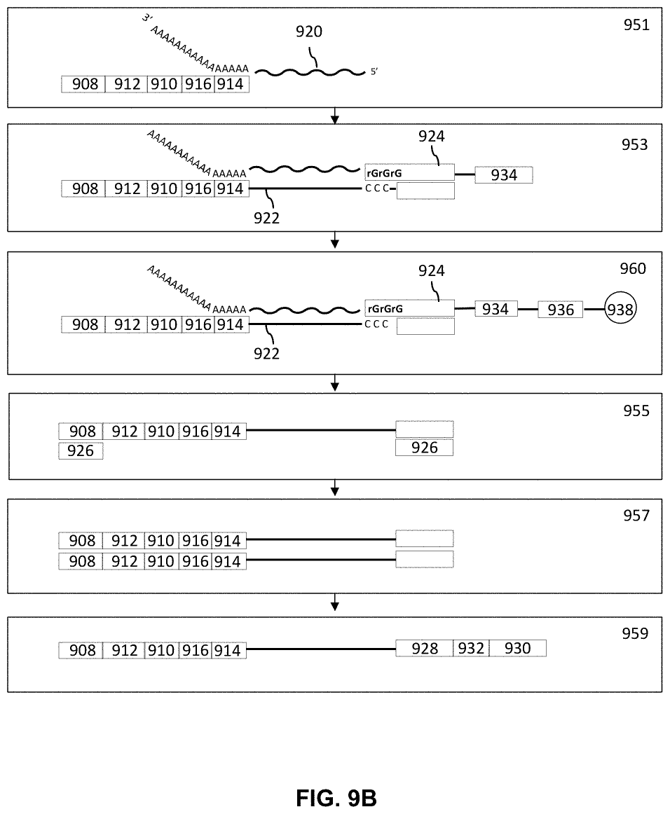

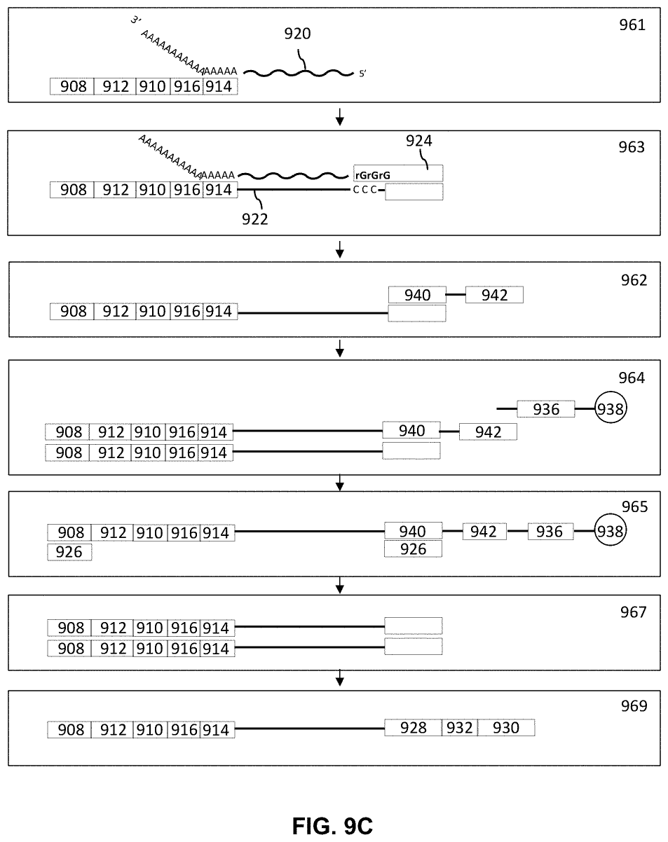

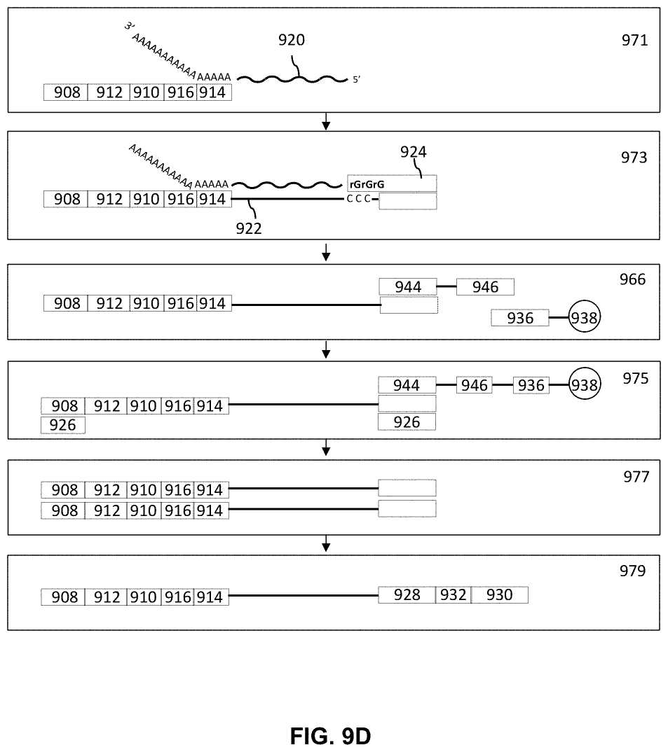

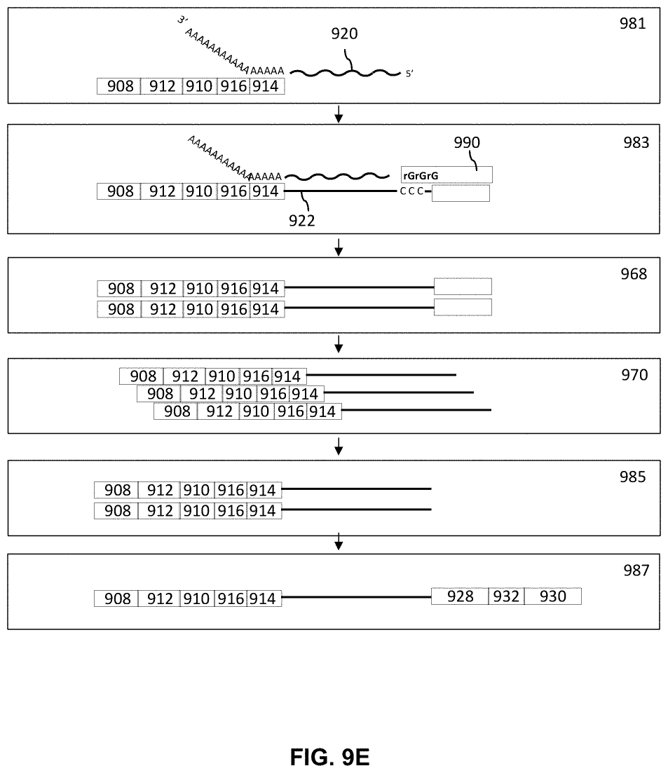

An additional example of a barcode oligonucleotide for use in RNA analysis, including messenger RNA (mRNA, including mRNA obtained from a cell) analysis, is shown in FIG. 9A. As shown, the overall oligonucleotide 902 can be coupled to a bead 904 by a releasable linkage 906, such as a disulfide linker. The oligonucleotide may include functional sequences that are used in subsequent processing, such as functional sequence 908, which may include a sequencer specific flow cell attachment sequence, e.g., a P5 sequence for Illumina sequencing systems, as well as functional sequence 910, which may include sequencing primer sequences, e.g., a R1 primer binding site for Illumina sequencing systems. A barcode sequence 912 is included within the structure for use in barcoding the sample RNA. An RNA specific (e.g., mRNA specific) priming sequence, such as poly-T sequence 914 is also included in the oligonucleotide structure. An anchoring sequence segment (not shown) may be included to ensure that the poly-T sequence hybridizes at the sequence end of the mRNA. An additional sequence segment 916 may be provided within the oligonucleotide sequence. This additional sequence can provide a unique molecular sequence segment, e.g., as a random N-mer sequence that varies across individual oligonucleotides coupled to a single bead, whereas barcode sequence 912 can be constant among oligonucleotides tethered to an individual bead. As described elsewhere herein, this unique sequence can serve to provide a unique identifier of the starting mRNA molecule that was captured, in order to allow quantitation of the number of original expressed RNA, e.g., mRNA counting. As will be appreciated, although shown as a single oligonucleotide tethered to the surface of a bead, individual beads can include tens to hundreds of thousands or even millions of individual oligonucleotide molecules, where, as noted, the barcode segment can be constant or relatively constant for a given bead, but where the variable or unique sequence segment will vary across an individual bead.

In an example method of cellular RNA (e.g., mRNA) analysis and in reference to FIG. 9A, a cell is co-partitioned along with a barcode bearing bead, switch oligo 924, and other reagents such as reverse transcriptase, a reducing agent and dNTPs into a partition (e.g., a droplet in an emulsion). In operation 950, the cell is lysed while the barcoded oligonucleotides 902 are released from the bead (e.g., via the action of the reducing agent) and the poly-T segment 914 of the released barcode oligonucleotide then hybridizes to the poly-A tail of mRNA 920 that is released from the cell. Next, in operation 952 the poly-T segment 914 is extended in a reverse transcription reaction using the mRNA as a template to produce a cDNA transcript 922 complementary to the mRNA and also includes each of the sequence segments 908, 912, 910, 916 and 914 of the barcode oligonucleotide. Terminal transferase activity of the reverse transcriptase can add additional bases to the cDNA transcript (e.g., polyC). The switch oligo 924 may then hybridize with the additional bases added to the cDNA transcript and facilitate template switching. A sequence complementary to the switch oligo sequence can then be incorporated into the cDNA transcript 922 via extension of the cDNA transcript 922 using the switch oligo 924 as a template. Within any given partition, all of the cDNA transcripts of the individual mRNA molecules will include a common barcode sequence segment 912. However, by including the unique random N-mer sequence 916, the transcripts made from different mRNA molecules within a given partition will vary at this unique sequence. As described elsewhere herein, this provides a quantitation feature that can be identifiable even following any subsequent amplification of the contents of a given partition, e.g., the number of unique segments associated with a common barcode can be indicative of the quantity of mRNA originating from a single partition, and thus, a single cell. Following operation 952, the cDNA transcript 922 is then amplified with primers 926 (e.g., PCR primers) in operation 954. Next, the amplified product is then purified (e.g., via solid phase reversible immobilization (SPRI)) in operation 956. At operation 958, the amplified product is then sheared, ligated to additional functional sequences, and further amplified (e.g., via PCR). The functional sequences may include a sequencer specific flow cell attachment sequence 930, e.g., a P7 sequence for Illumina sequencing systems, as well as functional sequence 928, which may include a sequencing primer binding site, e.g., for a R2 primer for Illumina sequencing systems, as well as functional sequence 932, which may include a sample index, e.g., an i7 sample index sequence for Illumina sequencing systems. In some cases, operations 950 and 952 can occur in the partition, while operations 954, 956 and 958 can occur in bulk solution (e.g., in a pooled mixture outside of the partition). In the case where a partition is a droplet in an emulsion, the emulsion can be broken and the contents of the droplet pooled in order to complete operations 954, 956 and 958. In some cases, operation 954 may be completed in the partition. In some cases, barcode oligonucleotides may be digested with exonucleases after the emulsion is broken. Exonuclease activity can be inhibited by ethylenediaminetetraacetic acid (EDTA) following primer digestion. Although described in terms of specific sequence references used for certain sequencing systems, e.g., Illumina systems, it will be understood that the reference to these sequences is for illustration purposes only, and the methods described herein may be configured for use with other sequencing systems incorporating specific priming, attachment, index, and other operational sequences used in those systems, e.g., systems available from Ion Torrent, Oxford Nanopore, Genia, Pacific Biosciences, Complete Genomics, and the like.

In an alternative example of a barcode oligonucleotide for use in RNA (e.g., cellular RNA) analysis as shown in FIG. 9A, functional sequence 908 may be a P7 sequence and functional sequence 910 may be a R2 primer binding site. Moreover, the functional sequence 930 may be a P5 sequence, functional sequence 928 may be a R1 primer binding site, and functional sequence 932 may be an i5 sample index sequence for Illumina sequencing systems. The configuration of the constructs generated by such a barcode oligonucleotide can help minimize (or avoid) sequencing of the poly-T sequence during sequencing.

Shown in FIG. 9B is another example method for RNA analysis, including cellular mRNA analysis. In this method, the switch oligo 924 is co-partitioned with the individual cell and barcoded bead along with reagents such as reverse transcriptase, a reducing agent and dNTPs into a partition (e.g., a droplet in an emulsion). The switch oligo 924 may be labeled with an additional tag 934, e.g. biotin. In operation 951, the cell is lysed while the barcoded oligonucleotides 902 (e.g., as shown in FIG. 9A) are released from the bead (e.g., via the action of the reducing agent). In some cases, sequence 908 is a P7 sequence and sequence 910 is a R2 primer binding site. In other cases, sequence 908 is a P5 sequence and sequence 910 is a R1 primer binding site. Next, the poly-T segment 914 of the released barcode oligonucleotide hybridizes to the poly-A tail of mRNA 920 that is released from the cell. In operation 953, the poly-T segment 914 is then extended in a reverse transcription reaction using the mRNA as a template to produce a cDNA transcript 922 complementary to the mRNA and also includes each of the sequence segments 908, 912, 910, 916 and 914 of the barcode oligonucleotide. Terminal transferase activity of the reverse transcriptase can add additional bases to the cDNA transcript (e.g., polyC). The switch oligo 924 may then hybridize with the cDNA transcript and facilitate template switching. A sequence complementary to the switch oligo sequence can then be incorporated into the cDNA transcript 922 via extension of the cDNA transcript 922 using the switch oligo 924 as a template. Next, an isolation operation 960 can be used to isolate the cDNA transcript 922 from the reagents and oligonucleotides in the partition. The additional tag 934, e.g. biotin, can be contacted with an interacting tag 936, e.g., streptavidin, which may be attached to a magnetic bead 938. At operation 960 the cDNA can be isolated with a pull-down operation (e.g., via magnetic separation, centrifugation) before amplification (e.g., via PCR) in operation 955, followed by purification (e.g., via solid phase reversible immobilization (SPRI)) in operation 957 and further processing (shearing, ligation of sequences 928, 932 and 930 and subsequent amplification (e.g., via PCR)) in operation 959. In some cases where sequence 908 is a P7 sequence and sequence 910 is a R2 primer binding site, sequence 930 is a P5 sequence and sequence 928 is a R1 primer binding site and sequence 932 is an i5 sample index sequence. In some cases where sequence 908 is a P5 sequence and sequence 910 is a R1 primer binding site, sequence 930 is a P7 sequence and sequence 928 is a R2 primer binding site and sequence 932 is an i7 sample index sequence. In some cases, as shown, operations 951 and 953 can occur in the partition, while operations 960, 955, 957 and 959 can occur in bulk solution (e.g., in a pooled mixture outside of the partition). In the case where a partition is a droplet in an emulsion, the emulsion can be broken and the contents of the droplet pooled in order to complete operation 960. The operations 955, 957, and 959 can then be carried out following operation 960 after the transcripts are pooled for processing.

Shown in FIG. 9C is another example method for RNA analysis, including cellular mRNA analysis. In this method, the switch oligo 924 is co-partitioned with the individual cell and barcoded bead along with reagents such as reverse transcriptase, a reducing agent and dNTPs in a partition (e.g., a droplet in an emulsion). In operation 961, the cell is lysed while the barcoded oligonucleotides 902 (e.g., as shown in FIG. 9A) are released from the bead (e.g., via the action of the reducing agent). In some cases, sequence 908 is a P7 sequence and sequence 910 is a R2 primer binding site. In other cases, sequence 908 is a P5 sequence and sequence 910 is a R1 primer binding site. Next, the poly-T segment 914 of the released barcode oligonucleotide then hybridizes to the poly-A tail of mRNA 920 that is released from the cell. Next, in operation 963 the poly-T segment 914 is then extended in a reverse transcription reaction using the mRNA as a template to produce a cDNA transcript 922 complementary to the mRNA and also includes each of the sequence segments 908, 912, 910, 916 and 914 of the barcode oligonucleotide. Terminal transferase activity of the reverse transcriptase can add additional bases to the cDNA transcript (e.g., polyC). The switch oligo 924 may then hybridize with the cDNA transcript and facilitate template switching. A sequence complementary to the switch oligo sequence can then be incorporated into the cDNA transcript 922 via extension of the cDNA transcript 922 using the switch oligo 924 as a template. Following operation 961 and operation 963, mRNA 920 and cDNA transcript 922 are denatured in operation 962. At operation 964, a second strand is extended from a primer 940 having an additional tag 942, e.g. biotin, and hybridized to the cDNA transcript 922. Also in operation 964, the biotin labeled second strand can be contacted with an interacting tag 936, e.g. streptavidin, which may be attached to a magnetic bead 938. The cDNA can be isolated with a pull-down operation (e.g., via magnetic separation, centrifugation) before amplification (e.g., via polymerase chain reaction (PCR)) in operation 965, followed by purification (e.g., via solid phase reversible immobilization (SPRI)) in operation 967 and further processing (shearing, ligation of sequences 928, 932 and 930 and subsequent amplification (e.g., via PCR)) in operation 969. In some cases where sequence 908 is a P7 sequence and sequence 910 is a R2 primer binding site, sequence 930 is a P5 sequence and sequence 928 is a R1 primer binding site and sequence 932 is an i5 sample index sequence. In some cases where sequence 908 is a P5 sequence and sequence 910 is a R1 primer binding site, sequence 930 is a P7 sequence and sequence 928 is a R2 primer binding site and sequence 932 is an i7 sample index sequence. In some cases, operations 961 and 963 can occur in the partition, while operations 962, 964, 965, 967, and 969 can occur in bulk (e.g., outside the partition). In the case where a partition is a droplet in an emulsion, the emulsion can be broken and the contents of the droplet pooled in order to complete operations 962, 964, 965, 967 and 969.

Shown in FIG. 9D is another example method for RNA analysis, including cellular mRNA analysis. In this method, the switch oligo 924 is co-partitioned with the individual cell and barcoded bead along with reagents such as reverse transcriptase, a reducing agent and dNTPs. In operation 971, the cell is lysed while the barcoded oligonucleotides 902 (e.g., as shown in FIG. 9A) are released from the bead (e.g., via the action of the reducing agent). In some cases, sequence 908 is a P7 sequence and sequence 910 is a R2 primer binding site. In other cases, sequence 908 is a P5 sequence and sequence 910 is a R1 primer binding site. Next the poly-T segment 914 of the released barcode oligonucleotide then hybridizes to the poly-A tail of mRNA 920 that is released from the cell. Next in operation 973, the poly-T segment 914 is then extended in a reverse transcription reaction using the mRNA as a template to produce a cDNA transcript 922 complementary to the mRNA and also includes each of the sequence segments 908, 912, 910, 916 and 914 of the barcode oligonucleotide. Terminal transferase activity of the reverse transcriptase can add additional bases to the cDNA transcript (e.g., polyC). The switch oligo 924 may then hybridize with the cDNA transcript and facilitate template switching. A sequence complementary to the switch oligo sequence can then be incorporated into the cDNA transcript 922 via extension of the cDNA transcript 922 using the switch oligo 924 as a template. In operation 966, the mRNA 920, cDNA transcript 922 and switch oligo 924 can be denatured, and the cDNA transcript 922 can be hybridized with a capture oligonucleotide 944 labeled with an additional tag 946, e.g. biotin. In this operation, the biotin-labeled capture oligonucleotide 944, which is hybridized to the cDNA transcript, can be contacted with an interacting tag 936, e.g. streptavidin, which may be attached to a magnetic bead 938. Following separation from other species (e.g., excess barcoded oligonucleotides) using a pull-down operation (e.g., via magnetic separation, centrifugation), the cDNA transcript can be amplified (e.g., via PCR) with primers 926 at operation 975, followed by purification (e.g., via solid phase reversible immobilization (SPRI)) in operation 977 and further processing (shearing, ligation of sequences 928, 932 and 930 and subsequent amplification (e.g., via PCR)) in operation 979. In some cases where sequence 908 is a P7 sequence and sequence 910 is a R2 primer binding site, sequence 930 is a P5 sequence and sequence 928 is a R1 primer binding site and sequence 932 is an i5 sample index sequence. In other cases where sequence 908 is a P5 sequence and sequence 910 is a R1 primer binding site, sequence 930 is a P7 sequence and sequence 928 is a R2 primer binding site and sequence 932 is an i7 sample index sequence. In some cases, operations 971 and 973 can occur in the partition, while operations 966, 975, 977 (purification), and 979 can occur in bulk (e.g., outside the partition). In the case where a partition is a droplet in an emulsion, the emulsion can be broken and the contents of the droplet pooled in order to complete operations 966, 975, 977 and 979.

Shown in FIG. 9E is another example method for RNA analysis, including cellular RNA analysis. In this method, an individual cell is co-partitioned along with a barcode bearing bead, a switch oligo 990, and other reagents such as reverse transcriptase, a reducing agent and dNTPs into a partition (e.g., a droplet in an emulsion). In operation 981, the cell is lysed while the barcoded oligonucleotides (e.g., 902 as shown in FIG. 9A) are released from the bead (e.g., via the action of the reducing agent). In some cases, sequence 908 is a P7 sequence and sequence 910 is a R2 primer binding site. In other cases, sequence 908 is a P5 sequence and sequence 910 is a R1 primer binding site. Next, the poly-T segment of the released barcode oligonucleotide then hybridizes to the poly-A tail of mRNA 920 released from the cell. Next at operation 983, the poly-T segment is then extended in a reverse transcription reaction to produce a cDNA transcript 922 complementary to the mRNA and also includes each of the sequence segments 908, 912, 910, 916 and 914 of the barcode oligonucleotide. Terminal transferase activity of the reverse transcriptase can add additional bases to the cDNA transcript (e.g., polyC). The switch oligo 990 may then hybridize with the cDNA transcript and facilitate template switching. A sequence complementary to the switch oligo sequence and including a T7 promoter sequence, can be incorporated into the cDNA transcript 922. At operation 968, a second strand is synthesized and at operation 970 the T7 promoter sequence can be used by T7 polymerase to produce RNA transcripts in in vitro transcription. At operation 985 the RNA transcripts can be purified (e.g., via solid phase reversible immobilization (SPRI)), reverse transcribed to form DNA transcripts, and a second strand can be synthesized for each of the DNA transcripts. In some cases, prior to purification, the RNA transcripts can be contacted with a DNase (e.g., DNAase I) to break down residual DNA. At operation 987 the DNA transcripts are then fragmented and ligated to additional functional sequences, such as sequences 928, 932 and 930 and, in some cases, further amplified (e.g., via PCR). In some cases where sequence 908 is a P7 sequence and sequence 910 is a R2 primer binding site, sequence 930 is a P5 sequence and sequence 928 is a R1 primer binding site and sequence 932 is an i5 sample index sequence. In some cases where sequence 908 is a P5 sequence and sequence 910 is a R1 primer binding site, sequence 930 is a P7 sequence and sequence 928 is a R2 primer binding site and sequence 932 is an i7 sample index sequence. In some cases, prior to removing a portion of the DNA transcripts, the DNA transcripts can be contacted with an RNase to break down residual RNA. In some cases, operations 981 and 983 can occur in the partition, while operations 968, 970, 985 and 987 can occur in bulk (e.g., outside the partition). In the case where a partition is a droplet in an emulsion, the emulsion can be broken and the contents of the droplet pooled in order to complete operations 968, 970, 985 and 987.

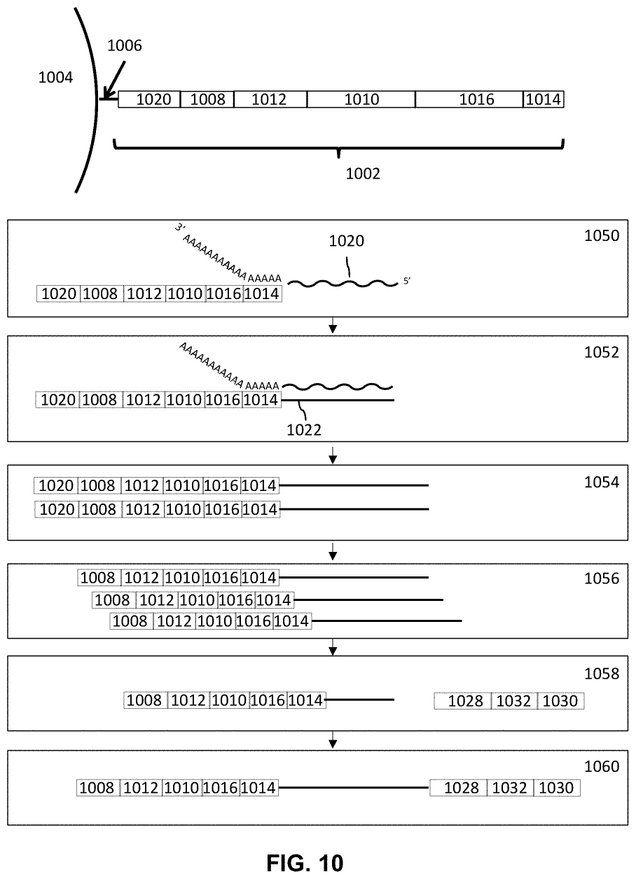

Another example of a barcode oligonucleotide for use in RNA analysis, including messenger RNA (mRNA, including mRNA obtained from a cell) analysis is shown in FIG. 10. As shown, the overall oligonucleotide 1002 is coupled to a bead 1004 by a releasable linkage 1006, such as a disulfide linker. The oligonucleotide may include functional sequences that are used in subsequent processing, such as functional sequence 1008, which may include a sequencer specific flow cell attachment sequence, e.g., a P7 sequence, as well as functional sequence 1010, which may include sequencing primer sequences, e.g., a R2 primer binding site. A barcode sequence 1012 is included within the structure for use in barcoding the sample RNA. An RNA specific (e.g., mRNA specific) priming sequence, such as poly-T sequence 1014 may be included in the oligonucleotide structure. An anchoring sequence segment (not shown) may be included to ensure that the poly-T sequence hybridizes at the sequence end of the mRNA. An additional sequence segment 1016 may be provided within the oligonucleotide sequence. This additional sequence can provide a unique molecular sequence segment, as described elsewhere herein. An additional functional sequence 1020 may be included for in vitro transcription, e.g., a T7 RNA polymerase promoter sequence. As will be appreciated, although shown as a single oligonucleotide tethered to the surface of a bead, individual beads can include tens to hundreds of thousands or even millions of individual oligonucleotide molecules, where, as noted, the barcode segment can be constant or relatively constant for a given bead, but where the variable or unique sequence segment will vary across an individual bead.

In an example method of cellular RNA analysis and in reference to FIG. 10, a cell is co-partitioned along with a barcode bearing bead, and other reagents such as reverse transcriptase, reducing agent and dNTPs into a partition (e.g., a droplet in an emulsion). In operation 1050, the cell is lysed while the barcoded oligonucleotides 1002 are released (e.g., via the action of the reducing agent) from the bead, and the poly-T segment 1014 of the released barcode oligonucleotide then hybridizes to the poly-A tail of mRNA 1020. Next at operation 1052, the poly-T segment is then extended in a reverse transcription reaction using the mRNA as template to produce a cDNA transcript 1022 of the mRNA and also includes each of the sequence segments 1020, 1008, 1012, 1010, 1016, and 1014 of the barcode oligonucleotide. Within any given partition, all of the cDNA transcripts of the individual mRNA molecules will include a common barcode sequence segment 1012. However, by including the unique random N-mer sequence, the transcripts made from different mRNA molecules within a given partition will vary at this unique sequence. As described elsewhere herein, this provides a quantitation feature that can be identifiable even following any subsequent amplification of the contents of a given partition, e.g., the number of unique segments associated with a common barcode can be indicative of the quantity of mRNA originating from a single partition, and thus, a single cell. At operation 1054 a second strand is synthesized and at operation 1056 the T7 promoter sequence can be used by T7 polymerase to produce RNA transcripts in in vitro transcription. At operation 1058 the transcripts are fragmented (e.g., sheared), ligated to additional functional sequences, and reverse transcribed. The functional sequences may include a sequencer specific flow cell attachment sequence 1030, e.g., a P5 sequence, as well as functional sequence 1028, which may include sequencing primers, e.g., a R1 primer binding sequence, as well as functional sequence 1032, which may include a sample index, e.g., an i5 sample index sequence. At operation 1060 the RNA transcripts can be reverse transcribed to DNA, the DNA amplified (e.g., via PCR), and sequenced to identify the sequence of the cDNA transcript of the mRNA, as well as to sequence the barcode segment and the unique sequence segment. In some cases, operations 1050 and 1052 can occur in the partition, while operations 1054, 1056, 1058 and 1060 can occur in bulk (e.g., outside the partition). In the case where a partition is a droplet in an emulsion, the emulsion can be broken and the contents of the droplet pooled in order to complete operations 1054, 1056, 1058 and 1060.

In an alternative example of a barcode oligonucleotide for use in RNA (e.g., cellular RNA) analysis as shown in FIG. 10, functional sequence 1008 may be a P5 sequence and functional sequence 1010 may be a R1 primer binding site. Moreover, the functional sequence 1030 may be a P7 sequence, functional sequence 1028 may be a R2 primer binding site, and functional sequence 1032 may be an i7 sample index sequence.

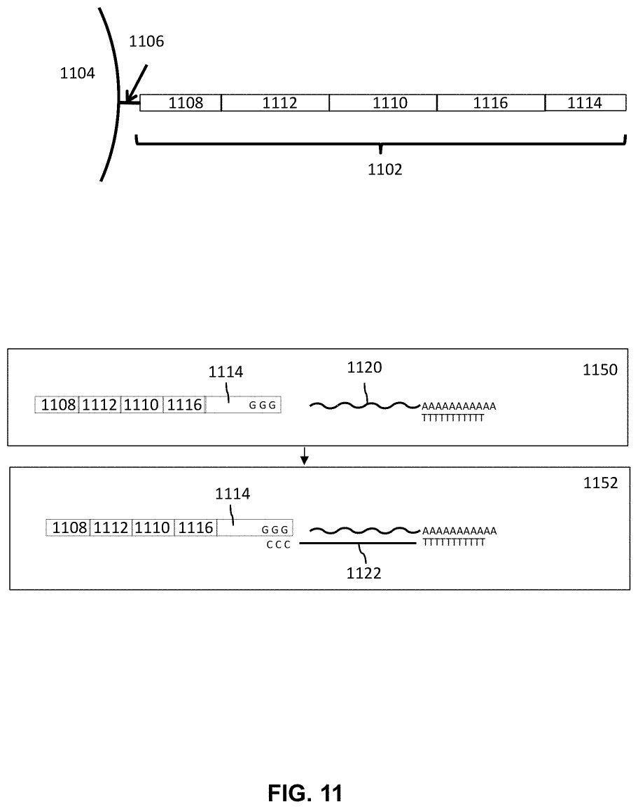

An additional example of a barcode oligonucleotide for use in RNA analysis, including messenger RNA (mRNA, including mRNA obtained from a cell) analysis is shown in FIG. 11. As shown, the overall oligonucleotide 1102 is coupled to a bead 1104 by a releasable linkage 1106, such as a disulfide linker. The oligonucleotide may include functional sequences that are used in subsequent processing, such as functional sequence 1108, which may include a sequencer specific flow cell attachment sequence, e.g., a P5 sequence, as well as functional sequence 1110, which may include sequencing primer sequences, e.g., a R1 primer binding site. In some cases, sequence 1108 is a P7 sequence and sequence 1110 is a R2 primer binding site. A barcode sequence 1112 is included within the structure for use in barcoding the sample RNA. An additional sequence segment 1116 may be provided within the oligonucleotide sequence. In some cases, this additional sequence can provide a unique molecular sequence segment, as described elsewhere herein. An additional sequence 1114 may be included to facilitate template switching, e.g., polyG. As will be appreciated, although shown as a single oligonucleotide tethered to the surface of a bead, individual beads can include tens to hundreds of thousands or even millions of individual oligonucleotide molecules, where, as noted, the barcode segment can be constant or relatively constant for a given bead, but where the variable or unique sequence segment will vary across an individual bead.

In an example method of cellular mRNA analysis and in reference to FIG. 11, a cell is co-partitioned along with a barcode bearing bead, poly-T sequence, and other reagents such as reverse transcriptase, a reducing agent and dNTPs into a partition (e.g., a droplet in an emulsion). In operation 1150, the cell is lysed while the barcoded oligonucleotides are released from the bead (e.g., via the action of the reducing agent) and the poly-T sequence hybridizes to the poly-A tail of mRNA 1120 released from the cell. Next, in operation 1152, the poly-T sequence is then extended in a reverse transcription reaction using the mRNA as a template to produce a cDNA transcript 1122 complementary to the mRNA. Terminal transferase activity of the reverse transcriptase can add additional bases to the cDNA transcript (e.g., polyC). The additional bases added to the cDNA transcript, e.g., polyC, can then to hybridize with 1114 of the barcoded oligonucleotide. This can facilitate template switching and a sequence complementary to the barcode oligonucleotide can be incorporated into the cDNA transcript. The transcripts can be further processed (e.g., amplified, portions removed, additional sequences added, etc.) and characterized as described elsewhere herein, e.g., by sequencing. The configuration of the constructs generated by such a method can help minimize (or avoid) sequencing of the poly-T sequence during sequencing.

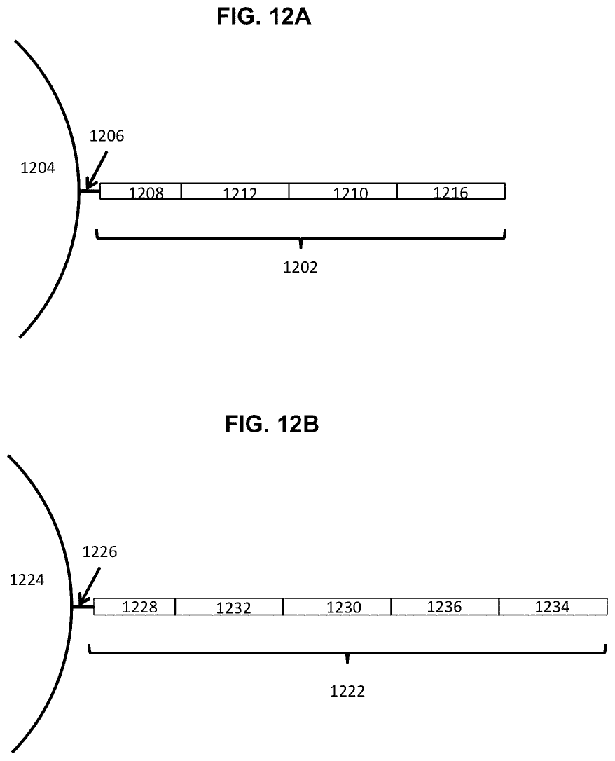

An additional example of a barcode oligonucleotide for use in RNA analysis, including cellular RNA analysis is shown in FIG. 12A. As shown, the overall oligonucleotide 1202 is coupled to a bead 1204 by a releasable linkage 1206, such as a disulfide linker. The oligonucleotide may include functional sequences that are used in subsequent processing, such as functional sequence 1208, which may include a sequencer specific flow cell attachment sequence, e.g., a P5 sequence, as well as functional sequence 1210, which may include sequencing primer sequences, e.g., a R1 primer binding site. In some cases, sequence 1208 is a P7 sequence and sequence 1210 is a R2 primer binding site. A barcode sequence 1212 is included within the structure for use in barcoding the sample RNA. An additional sequence segment 1216 may be provided within the oligonucleotide sequence. In some cases, this additional sequence can provide a unique molecular sequence segment, as described elsewhere herein. As will be appreciated, although shown as a single oligonucleotide tethered to the surface of a bead, individual beads can include tens to hundreds of thousands or even millions of individual oligonucleotide molecules, where, as noted, the barcode segment can be constant or relatively constant for a given bead, but where the variable or unique sequence segment will vary across an individual bead. In an example method of cellular RNA analysis using this barcode, a cell is co-partitioned along with a barcode bearing bead and other reagents such as RNA ligase and a reducing agent into a partition (e.g. a droplet in an emulsion). The cell is lysed while the barcoded oligonucleotides are released (e.g., via the action of the reducing agent) from the bead. The barcoded oligonucleotides can then be ligated to the 5′ end of mRNA transcripts while in the partitions by RNA ligase. Subsequent operations may include purification (e.g., via solid phase reversible immobilization (SPRI)) and further processing (shearing, ligation of functional sequences, and subsequent amplification (e.g., via PCR)), and these operations may occur in bulk (e.g., outside the partition). In the case where a partition is a droplet in an emulsion, the emulsion can be broken and the contents of the droplet pooled for the additional operations.

An additional example of a barcode oligonucleotide for use in RNA analysis, including cellular RNA analysis is shown in FIG. 12B. As shown, the overall oligonucleotide 1222 is coupled to a bead 1224 by a releasable linkage 1226, such as a disulfide linker. The oligonucleotide may include functional sequences that are used in subsequent processing, such as functional sequence 1228, which may include a sequencer specific flow cell attachment sequence, e.g., a P5 sequence, as well as functional sequence 1230, which may include sequencing primer sequences, e.g., a R1 primer binding site. In some cases, sequence 1228 is a P7 sequence and sequence 1230 is a R2 primer binding site. A barcode sequence 1232 is included within the structure for use in barcoding the sample RNA. A priming sequence 1234 (e.g., a random priming sequence) can also be included in the oligonucleotide structure, e.g., a random hexamer. An additional sequence segment 1236 may be provided within the oligonucleotide sequence. In some cases, this additional sequence provides a unique molecular sequence segment, as described elsewhere herein. As will be appreciated, although shown as a single oligonucleotide tethered to the surface of a bead, individual beads can include tens to hundreds of thousands or even millions of individual oligonucleotide molecules, where, as noted, the barcode segment can be constant or relatively constant for a given bead, but where the variable or unique sequence segment will vary across an individual bead. In an example method of cellular mRNA analysis using the barcode oligonucleotide of FIG. 12B, a cell is co-partitioned along with a barcode bearing bead and additional reagents such as reverse transcriptase, a reducing agent and dNTPs into a partition (e.g., a droplet in an emulsion). The cell is lysed while the barcoded oligonucleotides are released from the bead (e.g., via the action of the reducing agent). In some cases, sequence 1228 is a P7 sequence and sequence 1230 is a R2 primer binding site. In other cases, sequence 1228 is a P5 sequence and sequence 1230 is a R1 primer binding site. The priming sequence 1234 of random hexamers can randomly hybridize cellular mRNA. The random hexamer sequence can then be extended in a reverse transcription reaction using mRNA from the cell as a template to produce a cDNA transcript complementary to the mRNA and also includes each of the sequence segments 1228, 1232, 1230, 1236, and 1234 of the barcode oligonucleotide. Subsequent operations may include purification (e.g., via solid phase reversible immobilization (SPRI)), further processing (shearing, ligation of functional sequences, and subsequent amplification (e.g., via PCR)), and these operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA and cDNA from cells. Alternatively, rRNA removal agents may be applied during additional processing operations. The configuration of the constructs generated by such a method can help minimize (or avoid) sequencing of the poly-T sequence during sequencing.

The single cell analysis methods described herein may also be useful in the analysis of the whole transcriptome. Referring back to the barcode of FIG. 12B, the priming sequence 1234 may be a random N-mer. In some cases, sequence 1228 is a P7 sequence and sequence 1230 is a R2 primer binding site. In other cases, sequence 1228 is a P5 sequence and sequence 1230 is a R1 primer binding site. In an example method of whole transcriptome analysis using this barcode, the individual cell is co-partitioned along with a barcode bearing bead, poly-T sequence, and other reagents such as reverse transcriptase, polymerase, a reducing agent and dNTPs into a partition (e.g., droplet in an emulsion). In an operation of this method, the cell is lysed while the barcoded oligonucleotides are released from the bead (e.g., via the action of the reducing agent) and the poly-T sequence hybridizes to the poly-A tail of cellular mRNA. In a reverse transcription reaction using the mRNA as template, cDNA transcripts of cellular mRNA can be produced. The RNA can then be degraded with an RNase. The priming sequence 1234 in the barcoded oligonucleotide can then randomly hybridize to the cDNA transcripts. The oligonucleotides can be extended using polymerase enzymes and other extension reagents co-partitioned with the bead and cell similar to as shown in FIG. 3 to generate amplification products (e.g., barcoded fragments), similar to the example amplification product shown in FIG. 3 (panel F). The barcoded nucleic acid fragments may, in some cases subjected to further processing (e.g., amplification, addition of additional sequences, clean up processes, etc. as described elsewhere herein) characterized, e.g., through sequence analysis. In this operation, sequencing signals can come from full length RNA.

Although operations with various barcode designs have been discussed individually, individual beads can include barcode oligonucleotides of various designs for simultaneous use.

In addition to characterizing individual cells or cell sub-populations from larger populations, the processes and systems described herein may also be used to characterize individual cells as a way to provide an overall profile of a cellular, or other organismal population. A variety of applications require the evaluation of the presence and quantification of different cell or organism types within a population of cells, including, for example, microbiome analysis and characterization, environmental testing, food safety testing, epidemiological analysis, e.g., in tracing contamination or the like. In particular, the analysis processes described above may be used to individually characterize, sequence and/or identify large numbers of individual cells within a population. This characterization may then be used to assemble an overall profile of the originating population, which can provide important prognostic and diagnostic information.

For example, shifts in human microbiomes, including, e.g., gut, buccal, epidermal microbiomes, etc., have been identified as being both diagnostic and prognostic of different conditions or general states of health. Using the single cell analysis methods and systems described herein, one can again, characterize, sequence and identify individual cells in an overall population, and identify shifts within that population that may be indicative of diagnostic ally relevant factors. By way of example, sequencing of bacterial 16S ribosomal RNA genes has been used as a highly accurate method for taxonomic classification of bacteria. Using the targeted amplification and sequencing processes described above can provide identification of individual cells within a population of cells. One may further quantify the numbers of different cells within a population to identify current states or shifts in states over time. See, e.g., Morgan et al, PLoS Comput. Biol., Ch. 12, December 2012, 8(12):e1002808, and Ram et al., Syst. Biol. Reprod. Med., June 2011, 57(3):162-170, each of which is incorporated herein by reference in its entirety for all purposes. Likewise, identification and diagnosis of infection or potential infection may also benefit from the single cell analyses described herein, e.g., to identify microbial species present in large mixes of other cells or other biological material, cells and/or nucleic acids, including the environments described above, as well as any other diagnostically relevant environments, e.g., cerebrospinal fluid, blood, fecal or intestinal samples, or the like.

The foregoing analyses may also be particularly useful in the characterization of potential drug resistance of different cells, e.g., cancer cells, bacterial pathogens, etc., through the analysis of distribution and profiling of different resistance markers/mutations across cell populations in a given sample. Additionally, characterization of shifts in these markers/mutations across populations of cells over time can provide valuable insight into the progression, alteration, prevention, and treatment of a variety of diseases characterized by such drug resistance issues.

Although described in terms of cells, it will be appreciated that any of a variety of individual biological organisms, or components of organisms are encompassed within this description, including, for example, cells, viruses, organelles, cellular inclusions, vesicles, or the like. Additionally, where referring to cells, it will be appreciated that such reference includes any type of cell, including without limitation prokaryotic cells, eukaryotic cells, bacterial, fungal, plant, mammalian, or other animal cell types, mycoplasmas, normal tissue cells, tumor cells, or any other cell type, whether derived from single cell or multicellular organisms.

Similarly, analysis of different environmental samples to profile the microbial organisms, viruses, or other biological contaminants that are present within such samples, can provide important information about disease epidemiology, and potentially aid in forecasting disease outbreaks, epidemics an pandemics.

As described above, the methods, systems and compositions described herein may also be used for analysis and characterization of other aspects of individual cells or populations of cells. In one example process, a sample is provided that contains cells that are to be analyzed and characterized as to their cell surface proteins. Also provided is a library of antibodies, antibody fragments, or other molecules having a binding affinity to the cell surface proteins or antigens (or other cell features) for which the cell is to be characterized (also referred to herein as cell surface feature binding groups). For ease of discussion, these affinity groups are referred to herein as binding groups. The binding groups can include a reporter molecule that is indicative of the cell surface feature to which the binding group binds. In particular, a binding group type that is specific to one type of cell surface feature will comprise a first reporter molecule, while a binding group type that is specific to a different cell surface feature will have a different reporter molecule associated with it. In some aspects, these reporter molecules will comprise oligonucleotide sequences. Oligonucleotide based reporter molecules provide advantages of being able to generate significant diversity in terms of sequence, while also being readily attachable to most biomolecules, e.g., antibodies, etc., as well as being readily detected, e.g., using sequencing or array technologies. In the example process, the binding groups include oligonucleotides attached to them. Thus, a first binding group type, e.g., antibodies to a first type of cell surface feature, will have associated with it a reporter oligonucleotide that has a first nucleotide sequence. Different binding group types, e.g., antibodies having binding affinity for other, different cell surface features, will have associated therewith reporter oligonucleotides that comprise different nucleotide sequences, e.g., having a partially or completely different nucleotide sequence. In some cases, for each type of cell surface feature binding group, e.g., antibody or antibody fragment, the reporter oligonucleotide sequence may be known and readily identifiable as being associated with the known cell surface feature binding group. These oligonucleotides may be directly coupled to the binding group, or they may be attached to a bead, molecular lattice, e.g., a linear, globular, cross-slinked, or other polymer, or other framework that is attached or otherwise associated with the binding group, which allows attachment of multiple reporter oligonucleotides to a single binding group.

In the case of multiple reporter molecules coupled to a single binding group, such reporter molecules can comprise the same sequence, or a particular binding group will include a known set of reporter oligonucleotide sequences. As between different binding groups, e.g., specific for different cell surface features, the reporter molecules can be different and attributable to the particular binding group.

Attachment of the reporter groups to the binding groups may be achieved through any of a variety of direct or indirect, covalent or non-covalent associations or attachments. For example, in the case of oligonucleotide reporter groups associated with antibody based binding groups, such oligonucleotides may be covalently attached to a portion of an antibody or antibody fragment using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available (See, e.g., Fang, et al., Fluoride-Cleavable Biotinylation Phosphoramidite for 5′-end-Labeling and Affinity Purification of Synthetic Oligonucleotides, Nucleic Acids Res. Jan. 15, 2003; 31(2):708-715, DNA 3′ End Biotinylation Kit, available from Thermo Scientific, the full disclosures of which are incorporated herein by reference in their entirety for all purposes). Likewise, protein and peptide biotinylation techniques have been developed and are readily available (See, e.g., U.S. Pat. No. 6,265,552, the full disclosures of which are incorporated herein by reference in their entirety for all purposes).

The reporter oligonucleotides may be provided having any of a range of different lengths, depending upon the diversity of reporter molecules desired or a given analysis, the sequence detection scheme employed, and the like. In some cases, these reporter sequences can be greater than about 5 nucleotides in length, greater than about 10 nucleotides in length, greater than about 20, 30, 40, 50, 60, 70, 80, 90, 100, 120, 150 or even 200 nucleotides in length. In some cases, these reporter nucleotides may be less than about 250 nucleotides in length, less than about 200, 180, 150, 120 100, 90, 80, 70, 60, 50, 40, or even 30 nucleotides in length. In many cases, the reporter oligonucleotides may be selected to provide barcoded products that are already sized, and otherwise configured to be analyzed on a sequencing system. For example, these sequences may be provided at a length that ideally creates sequenceable products of a desired length for particular sequencing systems. Likewise, these reporter oligonucleotides may include additional sequence elements, in addition to the reporter sequence, such as sequencer attachment sequences, sequencing primer sequences, amplification primer sequences, or the complements to any of these.

In operation, a cell-containing sample is incubated with the binding molecules and their associated reporter oligonucleotides, for any of the cell surface features desired to be analyzed. Following incubation, the cells are washed to remove unbound binding groups. Following washing, the cells are partitioned into separate partitions, e.g., droplets, along with the barcode carrying beads described above, where each partition includes a limited number of cells, e.g., in some cases, a single cell. Upon releasing the barcodes from the beads, they will prime the amplification and barcoding of the reporter oligonucleotides. As noted above, the barcoded replicates of the reporter molecules may additionally include functional sequences, such as primer sequences, attachment sequences or the like.

The barcoded reporter oligonucleotides are then subjected to sequence analysis to identify which reporter oligonucleotides bound to the cells within the partitions. Further, by also sequencing the associated barcode sequence, one can identify that a given cell surface feature likely came from the same cell as other, different cell surface features, whose reporter sequences include the same barcode sequence, i.e., they were derived from the same partition.

Based upon the reporter molecules that emanate from an individual partition based upon the presence of the barcode sequence, one may then create a cell surface profile of individual cells from a population of cells. Profiles of individual cells or populations of cells may be compared to profiles from other cells, e.g., ‘normal’ cells, to identify variations in cell surface features, which may provide diagnostically relevant information. In particular, these profiles may be particularly useful in the diagnosis of a variety of disorders that are characterized by variations in cell surface receptors, such as cancer and other disorders.

### VI. Devices and Systems

Also provided herein are the microfluidic devices used for partitioning the cells as described above. Such microfluidic devices can comprise channel networks for carrying out the partitioning process like those set forth in FIGS. 1 and 2. Examples of particularly useful microfluidic devices are described in U.S. Provisional Patent Application No. 61/977,804, filed Apr. 4, 2014, and incorporated herein by reference in its entirety for all purposes. Briefly, these microfluidic devices can comprise channel networks, such as those described herein, for partitioning cells into separate partitions, and co-partitioning such cells with oligonucleotide barcode library members, e.g., disposed on beads. These channel networks can be disposed within a solid body, e.g., a glass, semiconductor or polymer body structure in which the channels are defined, where those channels communicate at their termini with reservoirs for receiving the various input fluids, and for the ultimate deposition of the partitioned cells, etc., from the output of the channel networks. By way of example, and with reference to FIG. 2, a reservoir fluidly coupled to channel 202 may be provided with an aqueous suspension of cells 214, while a reservoir coupled to channel 204 may be provided with an aqueous suspension of beads 216 carrying the oligonucleotides. Channel segments 206 and 208 may be provided with a non-aqueous solution, e.g., an oil, into which the aqueous fluids are partitioned as droplets at the channel junction 212. Finally, an outlet reservoir may be fluidly coupled to channel 210 into which the partitioned cells and beads can be delivered and from which they may be harvested. As will be appreciated, while described as reservoirs, it will be appreciated that the channel segments may be coupled to any of a variety of different fluid sources or receiving components, including tubing, manifolds, or fluidic components of other systems.

Also provided are systems that control flow of these fluids through the channel networks e.g., through applied pressure differentials, centrifugal force, electrokinetic pumping, capillary or gravity flow, or the like.

### VII. Kits

Also provided herein are kits for analyzing individual cells or small populations of cells. The kits may include one, two, three, four, five or more, up to all of partitioning fluids, including both aqueous buffers and non-aqueous partitioning fluids or oils, nucleic acid barcode libraries that are releasably associated with beads, as described herein, microfluidic devices, reagents for disrupting cells amplifying nucleic acids, and providing additional functional sequences on fragments of cellular nucleic acids or replicates thereof, as well as instructions for using any of the foregoing in the methods described herein.

### VIII. Computer Control Systems

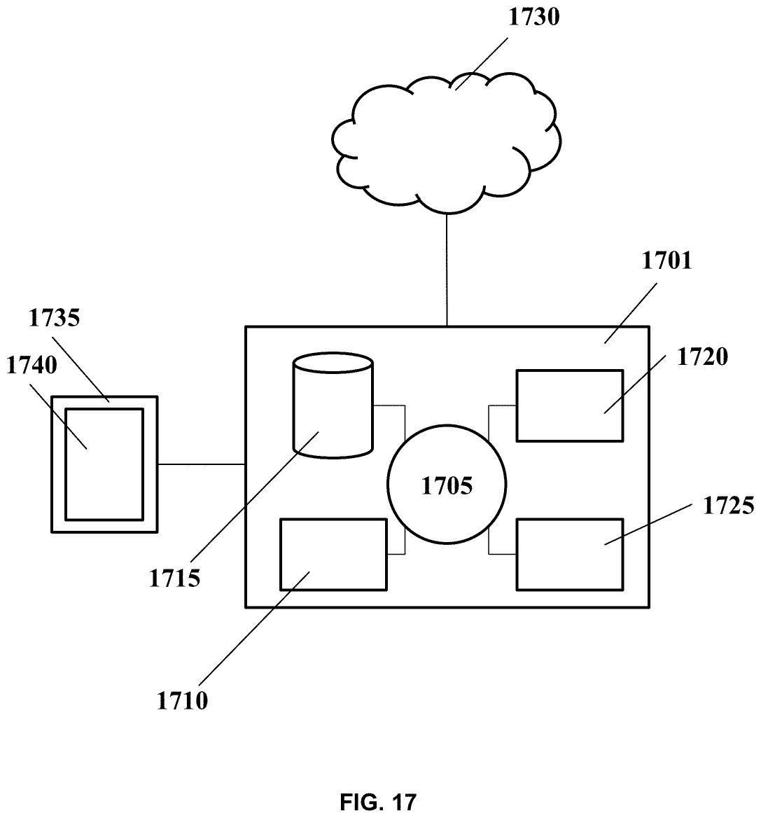

The present disclosure provides computer control systems that are programmed to implement methods of the disclosure. FIG. 17 shows a computer system 1701 that is programmed or otherwise configured to implement methods of the disclosure including nucleic acid sequencing methods, interpretation of nucleic acid sequencing data and analysis of cellular nucleic acids, such as RNA (e.g., mRNA), and characterization of cells from sequencing data. The computer system 1701 can be an electronic device of a user or a computer system that is remotely located with respect to the electronic device. The electronic device can be a mobile electronic device.

The computer system 1701 includes a central processing unit (CPU, also “processor” and “computer processor” herein) 1705, which can be a single core or multi core processor, or a plurality of processors for parallel processing. The computer system 1701 also includes memory or memory location 1710 (e.g., random-access memory, read-only memory, flash memory), electronic storage unit 1715 (e.g., hard disk), communication interface 1720 (e.g., network adapter) for communicating with one or more other systems, and peripheral devices 1725, such as cache, other memory, data storage and/or electronic display adapters. The memory 1710, storage unit 1715, interface 1720 and peripheral devices 1725 are in communication with the CPU 1705 through a communication bus (solid lines), such as a motherboard. The storage unit 1715 can be a data storage unit (or data repository) for storing data. The computer system 1701 can be operatively coupled to a computer network (“network”) 1730 with the aid of the communication interface 1720. The network 1730 can be the Internet, an internet and/or extranet, or an intranet and/or extranet that is in communication with the Internet. The network 1730 in some cases is a telecommunication and/or data network. The network 1730 can include one or more computer servers, which can enable distributed computing, such as cloud computing. The network 1730, in some cases with the aid of the computer system 1701, can implement a peer-to-peer network, which may enable devices coupled to the computer system 1701 to behave as a client or a server.

The CPU 1705 can execute a sequence of machine-readable instructions, which can be embodied in a program or software. The instructions may be stored in a memory location, such as the memory 1710. The instructions can be directed to the CPU 1705, which can subsequently program or otherwise configure the CPU 1705 to implement methods of the present disclosure. Examples of operations performed by the CPU 1705 can include fetch, decode, execute, and writeback.

The CPU 1705 can be part of a circuit, such as an integrated circuit. One or more other components of the system 1701 can be included in the circuit. In some cases, the circuit is an application specific integrated circuit (ASIC).

The storage unit 1715 can store files, such as drivers, libraries and saved programs. The storage unit 1715 can store user data, e.g., user preferences and user programs. The computer system 1701 in some cases can include one or more additional data storage units that are external to the computer system 1701, such as located on a remote server that is in communication with the computer system 1701 through an intranet or the Internet.

The computer system 1701 can communicate with one or more remote computer systems through the network 1730. For instance, the computer system 1701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1701 via the network 1730.

Methods as described herein can be implemented by way of machine (e.g., computer processor) executable code stored on an electronic storage location of the computer system 1701, such as, for example, on the memory 1710 or electronic storage unit 1715. The machine executable or machine readable code can be provided in the form of software. During use, the code can be executed by the processor 1705. In some cases, the code can be retrieved from the storage unit 1715 and stored on the memory 1710 for ready access by the processor 1705. In some situations, the electronic storage unit 1715 can be precluded, and machine-executable instructions are stored on memory 1710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1701 can include or be in communication with an electronic display 1735 that comprises a user interface (UI) 1740 for providing, for example, results of nucleic acid sequencing, analysis of nucleic acid sequencing data, characterization of nucleic acid sequencing samples, cell characterizations, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1705. The algorithm can, for example, initiate nucleic acid sequencing, process nucleic acid sequencing data, interpret nucleic acid sequencing results, characterize nucleic acid samples, characterize cells, etc.

### EXAMPLES

Various aspects of the disclosure are further illustrated by the following non-limiting examples.

### Example I: Cellular RNA Analysis Using Emulsions

In an example, reverse transcription with template switching and cDNA amplification (via PCR) is performed in emulsion droplets with operations as shown in FIG. 9A. The reaction mixture that is partitioned for reverse transcription and cDNA amplification (via PCR) includes 1,000 cells or 10,000 cells or 10 ng of RNA, beads bearing barcoded oligonucleotides/0.2% Tx-100/5× Kapa buffer, 2× Kapa HS HiFi Ready Mix, 4 μM switch oligo, and Smartscribe. Where cells are present, the mixture is partitioned such that a majority or all of the droplets comprise a single cell and single bead. The cells are lysed while the barcoded oligonucleotides are released from the bead, and the poly-T segment of the barcoded oligonucleotide hybridizes to the poly-A tail of mRNA that is released from the cell as in operation 950. The poly-T segment is extended in a reverse transcription reaction as in operation 952 and the cDNA transcript is amplified as in operation 954. The thermal cycling conditions are 42° C. for 130 minutes; 98° C. for 2 min; and 35 cycles of the following 98° C. for 15 sec, 60° C. for 20 sec, and 72° C. for 6 min. Following thermal cycling, the emulsion is broken and the transcripts are purified with Dynabeads and 0.6× SPRI as in operation 956.

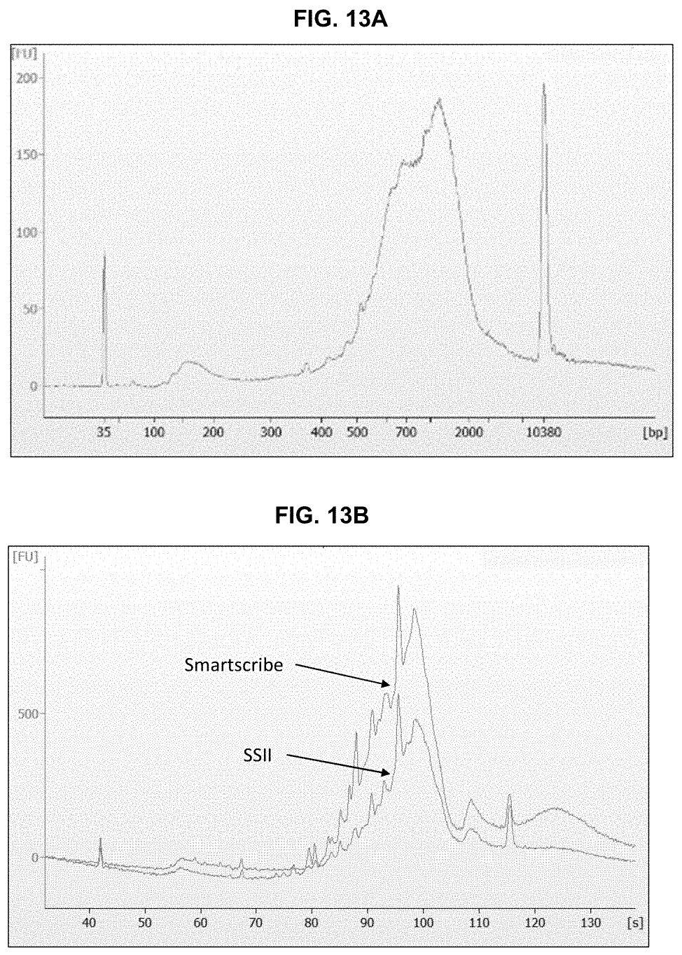

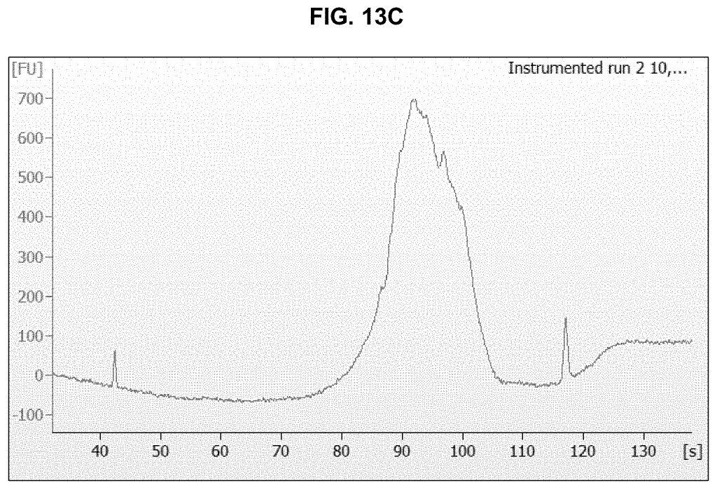

The yield from template switch reverse transcription and PCR in emulsions is shown for 1,000 cells in FIG. 13A and 10,000 cells in FIG. 13C and 10 ng of RNA in FIG. 13B (Smartscribe line). The cDNA transcripts from RT and PCR performed in emulsions for 10 ng RNA is sheared and ligated to functional sequences, cleaned up with 0.8×SPRI, and is further amplified by PCR as in operation 958. The amplification product is cleaned up with 0.8×SPRI. The yield from this processing is shown in FIG. 13B (SSII line).

### Example II: Cellular RNA Analysis Using Emulsions

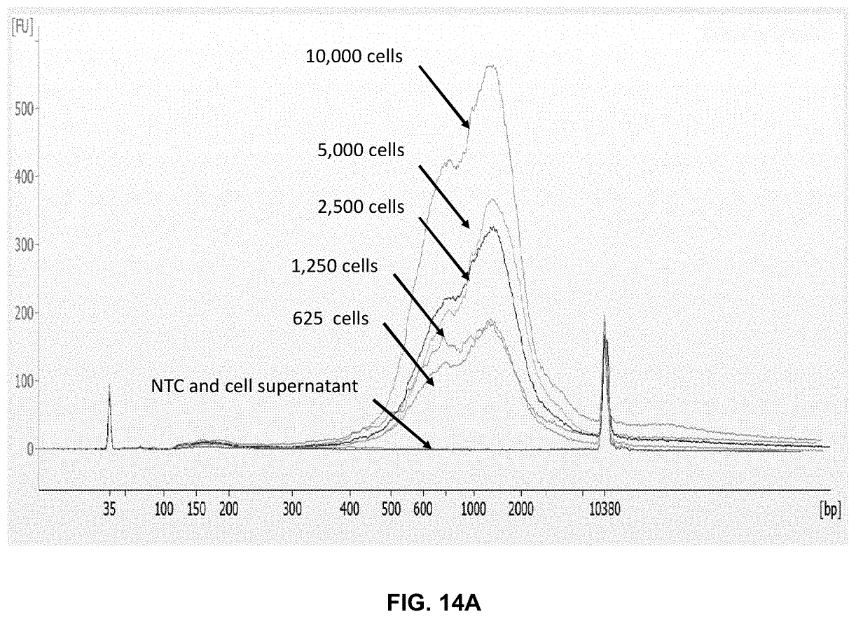

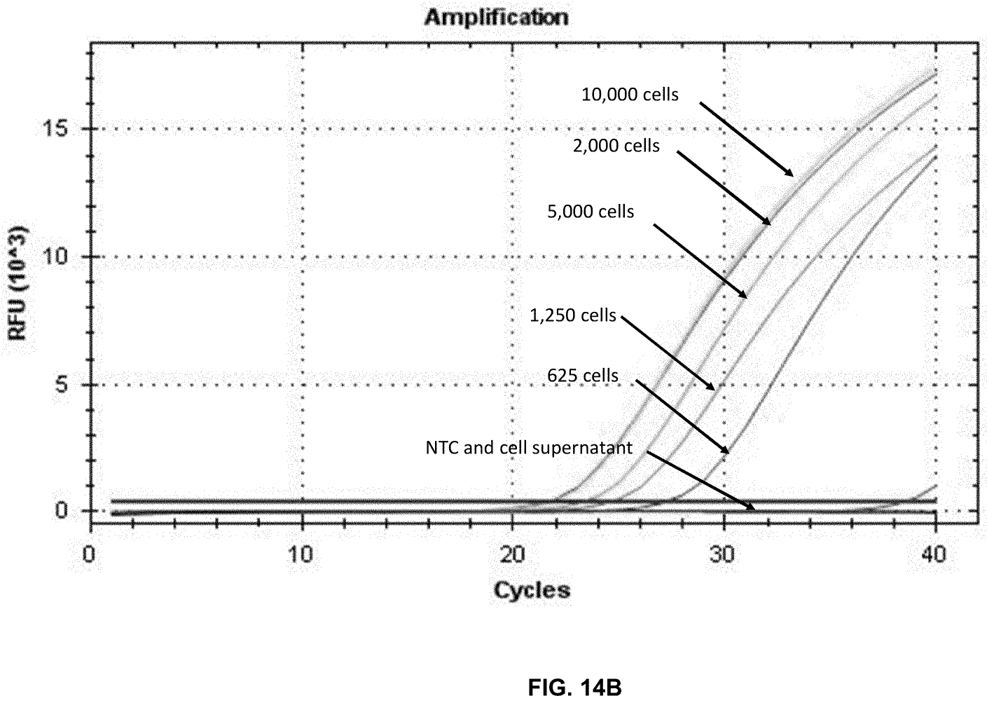

In another example, reverse transcription with template switching and cDNA amplification (via PCR) is performed in emulsion droplets with operations as shown in FIG. 9A. The reaction mixture that is partitioned for reverse transcription and cDNA amplification (via PCR) includes Jurkat cells, beads bearing barcoded oligonucleotides/0.2% TritonX-100/5× Kapa buffer, 2× Kapa HS HiFi Ready Mix, 4 μM switch oligo, and Smartscribe. The mixture is partitioned such that a majority or all of the droplets comprise a single cell and single bead. The cells are lysed while the barcoded oligonucleotides are released from the bead, and the poly-T segment of the barcoded oligonucleotide hybridizes to the poly-A tail of mRNA that is released from the cell as in operation 950. The poly-T segment is extended in a reverse transcription reaction as in operation 952 and the cDNA transcript is amplified as in operation 954. The thermal cycling conditions are 42° C. for 130 minutes; 98° C. for 2 min; and 35 cycles of the following 98° C. for 15 sec, 60° C. for 20 sec, and 72° C. for 6 min. Following thermal cycling, the emulsion is broken and the transcripts are cleaned-up with Dynabeads and 0.6×SPRI as in operation 956. The yield from reactions with various cell numbers (625 cells, 1,250 cells, 2,500 cells, 5,000 cells, and 10,000 cells) is shown in FIG. 14A. These yields are confirmed with GADPH qPCR assay results shown in FIG. 14B.

### Example III: RNA Analysis Using Emulsions

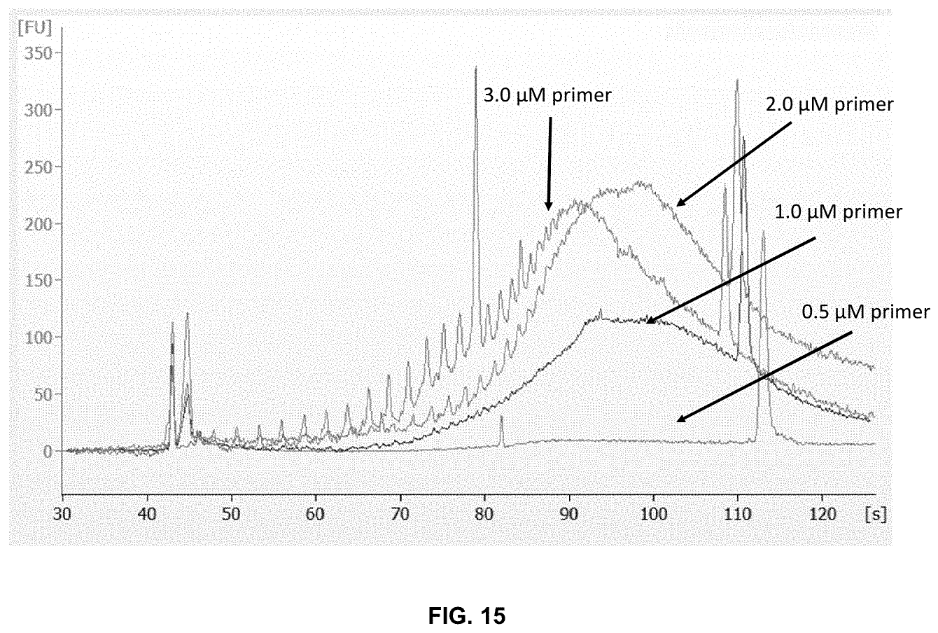

In another example, reverse transcription is performed in emulsion droplets and cDNA amplification is performed in bulk in a manner similar to that as shown in FIG. 9C. The reaction mixture that is partitioned for reverse transcription includes beads bearing barcoded oligonucleotides, 10 ng Jurkat RNA (e.g., Jurkat mRNA), 5× First-Strand buffer, and Smartscribe. The barcoded oligonucleotides are released from the bead, and the poly-T segment of the barcoded oligonucleotide hybridizes to the poly-A tail of the RNA as in operation 961. The poly-T segment is extended in a reverse transcription reaction as in operation 963. The thermal cycling conditions for reverse transcription are one cycle at 42° C. for 2 hours and one cycle at 70° C. for 10 min. Following thermal cycling, the emulsion is broken and RNA and cDNA transcripts are denatured as in operation 962. A second strand is then synthesized by primer extension with a primer having a biotin tag as in operation 964. The reaction conditions for this primer extension include cDNA transcript as the first strand and biotinylated extension primer ranging in concentration from 0.5-3.0 μM. The thermal cycling conditions are one cycle at 98° C. for 3 min and one cycle of 98° C. for 15 sec, 60° C. for 20 sec, and 72° C. for 30 min. Following primer extension, the second strand is pulled down with Dynabeads MyOne Streptavidin C1 and T1, and cleaned-up with Agilent SureSelect XT buffers. The second strand is pre-amplified via PCR as in operation 965 with the following cycling conditions—one cycle at 98° C. for 3 min and one cycle of 98° C. for 15 sec, 60° C. for 20 sec, and 72° C. for 30 min. The yield for various concentrations of biotinylated primer (0.5 μM, 1.0 μM, 2.0 μM, and 3.0 μM) is shown in FIG. 15.

### Example IV: RNA Analysis Using Emulsions

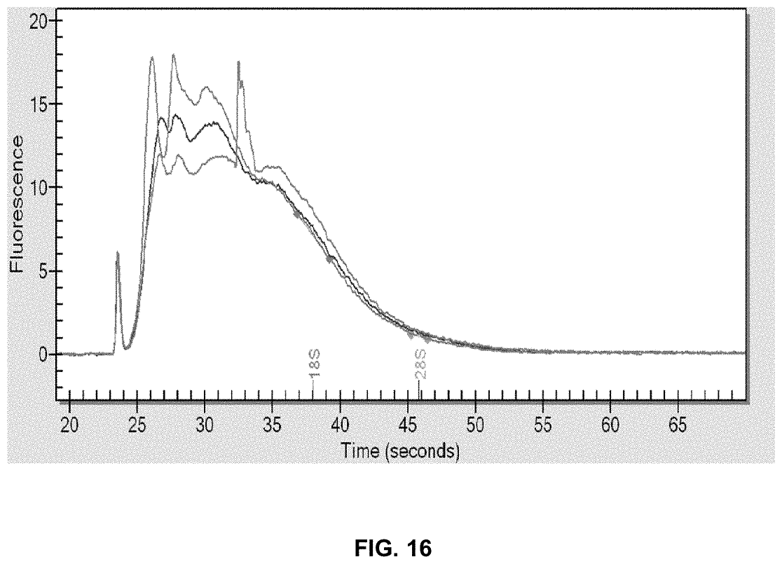

In another example, in vitro transcription by T7 polymerase is used to produce RNA transcripts as shown in FIG. 10. The mixture that is partitioned for reverse transcription includes beads bearing barcoded oligonucleotides which also include a T7 RNA polymerase promoter sequence, 10 ng human RNA (e.g., human mRNA), 5× First-Strand buffer, and Smartscribe. The mixture is partitioned such that a majority or all of the droplets comprise a single bead. The barcoded oligonucleotides are released from the bead, and the poly-T segment of the barcoded oligonucleotide hybridizes to the poly-A tail of the RNA as in operation 1050. The poly-T segment is extended in a reverse transcription reaction as in operation 1052. The thermal cycling conditions are one cycle at 42° C. for 2 hours and one cycle at 70° C. for 10 min. Following thermal cycling, the emulsion is broken and the remaining operations are performed in bulk. A second strand is then synthesized by primer extension as in operation 1054. The reaction conditions for this primer extension include cDNA transcript as template and extension primer. The thermal cycling conditions are one cycle at 98° C. for 3 min and one cycle of 98° C. for 15 sec, 60° C. for 20 sec, and 72° C. for 30 min. Following this primer extension, the second strand is purified with 0.6×SPRI. As in operation 1056, in vitro transcription is then performed to produce RNA transcripts. In vitro transcription is performed overnight, and the transcripts are purified with 0.6×SPRI. The RNA yields from in vitro transcription are shown in FIG. 16.

### Example V: Cell Population Analysis Using Single Nucleotide Polymorphisms (SNPs) from Single Cell Transcriptomes

A single cell platform capable of profiling expression of RNAs from tens of thousands of single cells can enable discovery of heterogeneity from populations of cells, for example, in nervous systems, developmental systems, and immune systems. Such a single cell platform can also be used to explore differences in compositions of cell populations among different individuals and species. One potential application is the study of graft vs host disease in transplantation studies, when cells from a donor are mixed with cells of a recipient. Existing methods of monitoring progress/status of transplantation include digital PCR, bulk RNA sequencing (RNA-seq), and flow cytometry. Digital PCR may be limited by the number of genes that can be examined at a time. Further, digital PCR may not allow the monitoring of populations over time or selection of subsets of populations for analysis. Bulk RNA-seq can average out the signal from all cells, thus potentially obscuring signals from a small subset or subsets of cell. Flow cytometry can separate cells based on cell surface markers, however, not every population may have accessible surface markers.

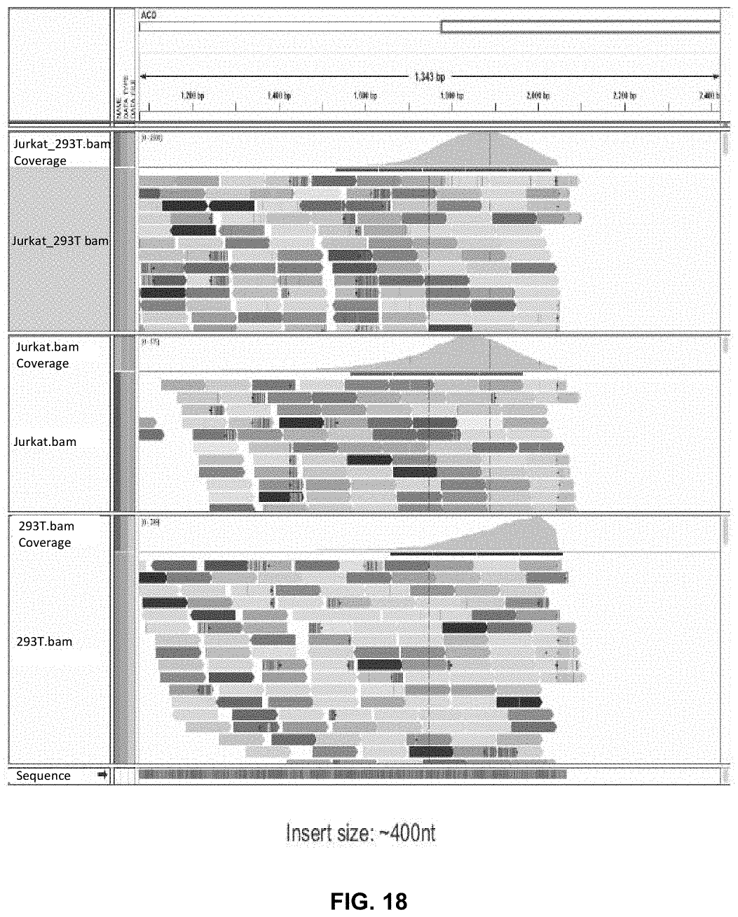

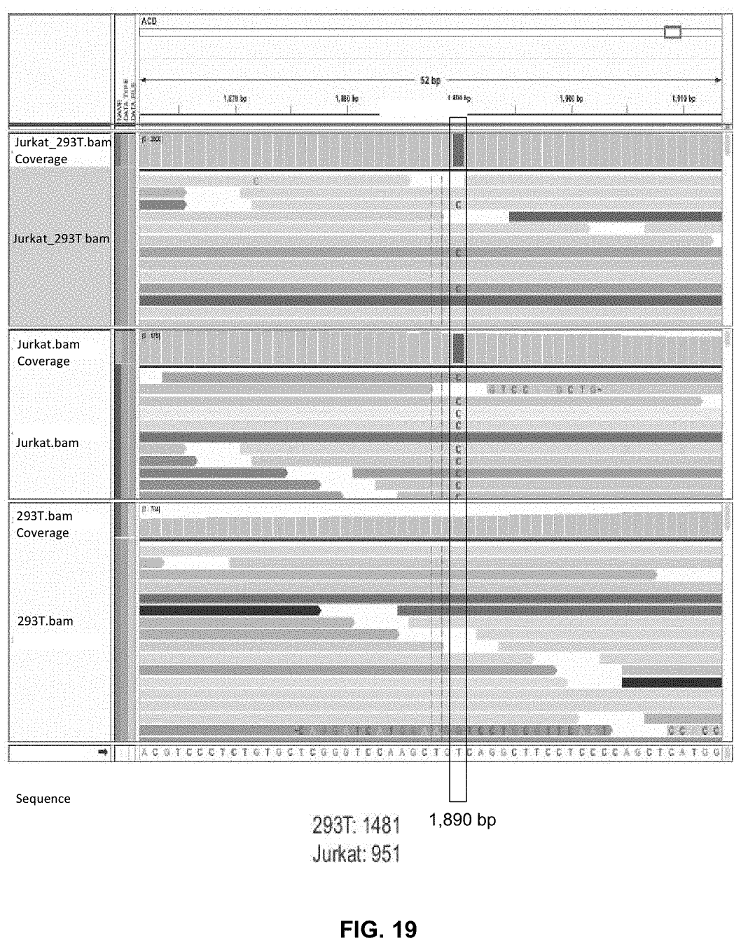

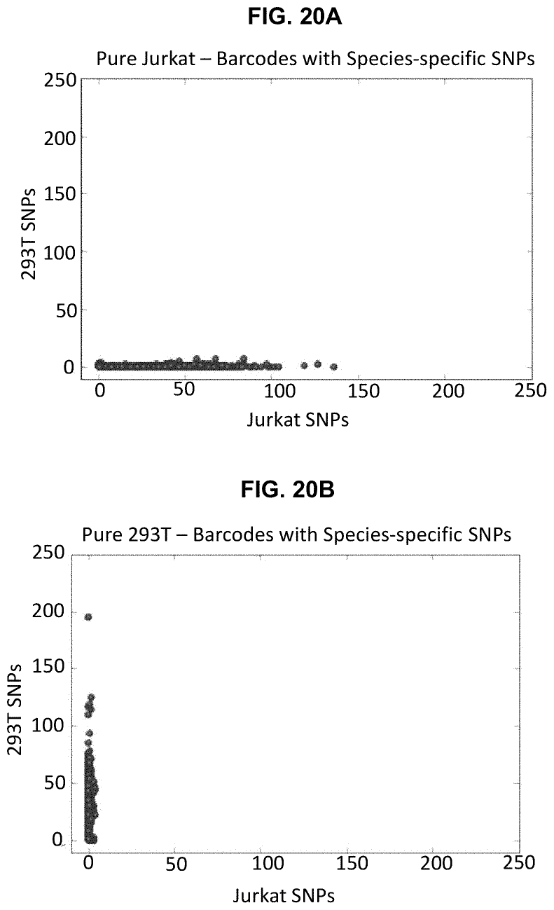

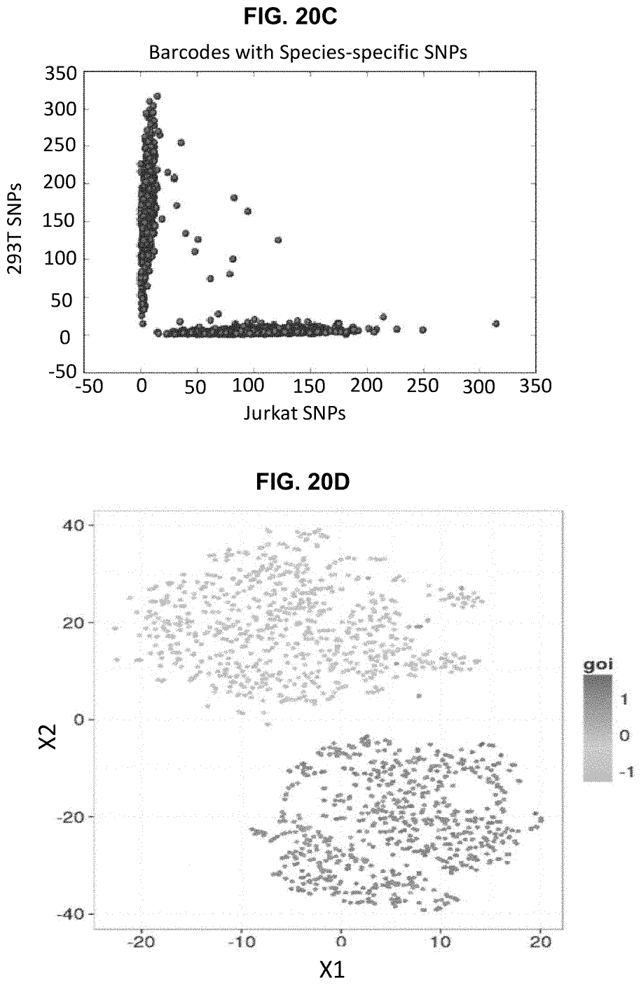

In this example, methods and systems herein were used to generate single cell RNA sequencing data (e.g., transcriptome), and the sequencing data was used to identify single nucleotide polymorphisms (SNPs). The SNPs identified were then used to distinguish cell populations. Briefly, single cell RNA sequencing data (e.g., transcriptome) was generated from samples comprising a mixture of HEK293T and Jurkat cells. SNPs were discovered from read sequences that mapped to the transcriptome. Although most reads clustered in the 3′ untranslated regions (UTRs) of genes, the insert length of ˜300-400 nt was sufficient to allow for variant calling (FIG. 18). Analysis of SNPs in HEK293T and Jurkat cells allowed species-specific SNPs to be identified (FIG. 19). FIGS. 20A and 20B show the distribution of cell-type specific SNPs (HEK293T and Jurkat). FIG. 20C shows the distribution of Jurkat-specific and 293T-specific SNPs in a Jurkat:293T mixed sample, specifically by SNPs in 3′ UTRs. FIG. 20D illustrates that Jurkat and 293T cells can be separated by Jurkat-specific marker gene CD3D.

### Example VI: Digital Transcriptional Profiling of Single Cells

In this example, the methods and systems herein were used for detecting single nucleotide polymorphisms (SNPs) with single cell RNA sequencing data (e.g., transcriptome). The SNPs identified were used to distinguish individuals/species, including but not limited to graft and host cells in transplantation.

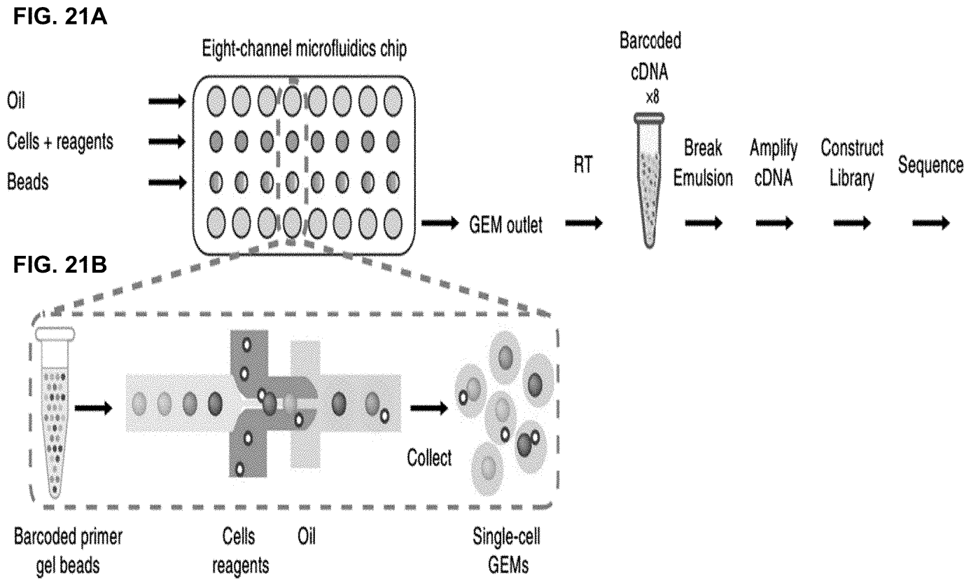

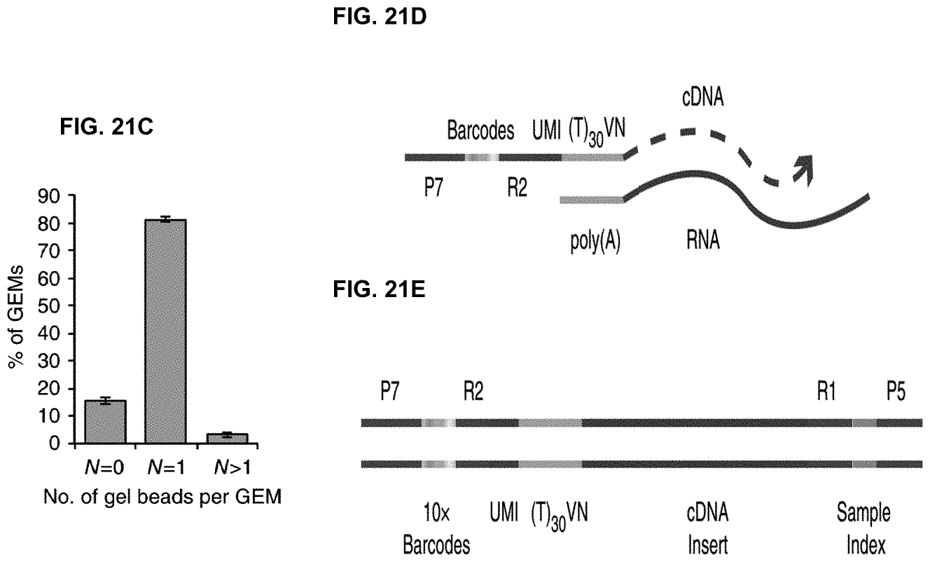

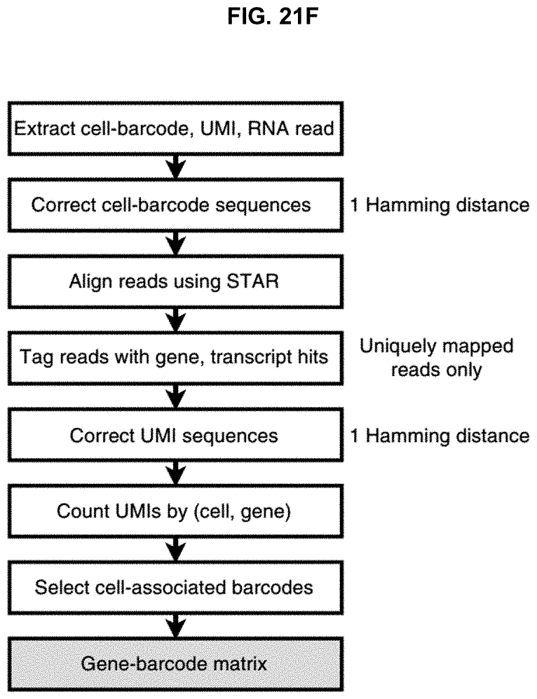

The droplet based microfluidic system in this example partitioned cells of a cell sample into droplets comprising gel beads. Partitions, or droplets, comprising cells and gel beads preferably contain one cell and one gel bead, but in some cases can contain various numbers of cells and various numbers of gel beads (including no cells or no gel beads). Briefly, droplets comprising gel beads (sometimes referred to herein as a GEM), were formed in an 8-channel microfluidic chip that encapsulates single gel beads at ˜80% fill rate (FIGS. 21A-C). As shown in FIGS. 21A and 21B, cells were combined with reagents in one channel of a microfluidic chip and then with gel beads from another channel to form GEMs. Reverse transcription (RT) was performed inside each GEM. Following RT, cDNAs were pooled for amplification and library construction in bulk. Each gel bead was functionalized with barcoded oligonucleotides comprising: i) sequencing adapters and primers, ii) a 14 bp barcode drawn from approximately 750,000 designed sequences to index GEMs, iii) a 10 bp randomer to index molecules (unique molecular identifier, UMI), and iv) a 30 bp oligo-dT (SEQ ID NO: 1) to prime poly-adenylated RNA transcripts (FIG. 21D). Within each microfluidic channel, 100,000 GEMs were formed per ˜6 min run, encapsulating thousands of cells in GEMs. Cells were loaded at a limiting dilution to minimize co-occurrence of multiple cells in the same GEM.

After encapsulation, cells were lysed and poly-adenylated RNAs were reverse transcribed. Each cDNA molecule produced contained a UMI and shared barcode per GEM, and ended with a template switching oligo at the 3′ end (FIG. 21E). Next, the droplets were broken and barcoded cDNA was pooled for PCR amplification. Primers complementary to the switch oligos and sequencing adapters were used. Finally, amplified cDNAs were sheared, and adapter and sample indices were incorporated into finished libraries which were compatible with next-generation short-read sequencing. Read1 contained the cDNA insert while Read2 captured the UMI. Index reads, 15 and 17, contained the sample indices and cell barcodes respectively. The streamlined approach described in this example enables parallel capture of thousands of cells in each of the 8 channels for scRNA-seq analysis.

A. Technical Demonstration with Cell Lines and Synthetic RNAs.

To assess the technical performance of the methods and systems described herein, a mixture of ˜1,200 human (293T) and ˜1,200 mouse (3T3) cells was loaded into the microfluidic system and processed as described above. The resulting library was sequenced on the Illumina NextSeq 500 and yielded ˜100 k reads/cell. Sequencing data were processed as illustrated in FIG. 21F. Briefly, 98-nt of Read1s were aligned against the union of human (hg19) and mouse (mm10) genomes with STAR. Barcodes and UMIs were filtered and corrected. PCR duplicates were marked using the barcode, UMI and gene ID. Only confidently mapped, non-PCR duplicates with valid barcodes and UMIs were used to generate the gene-barcode matrix for further analysis. Approximately 38% and 33% of reads mapped to human and mouse exonic regions, respectively and <6% of reads mapped to intronic regions. The mapping rate is comparable to previously reported scRNA-seq systems.

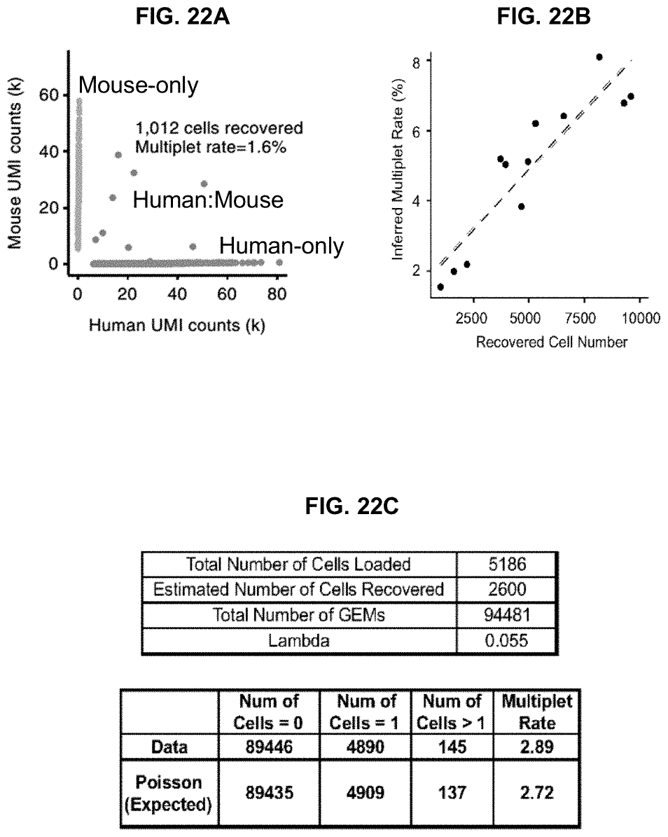

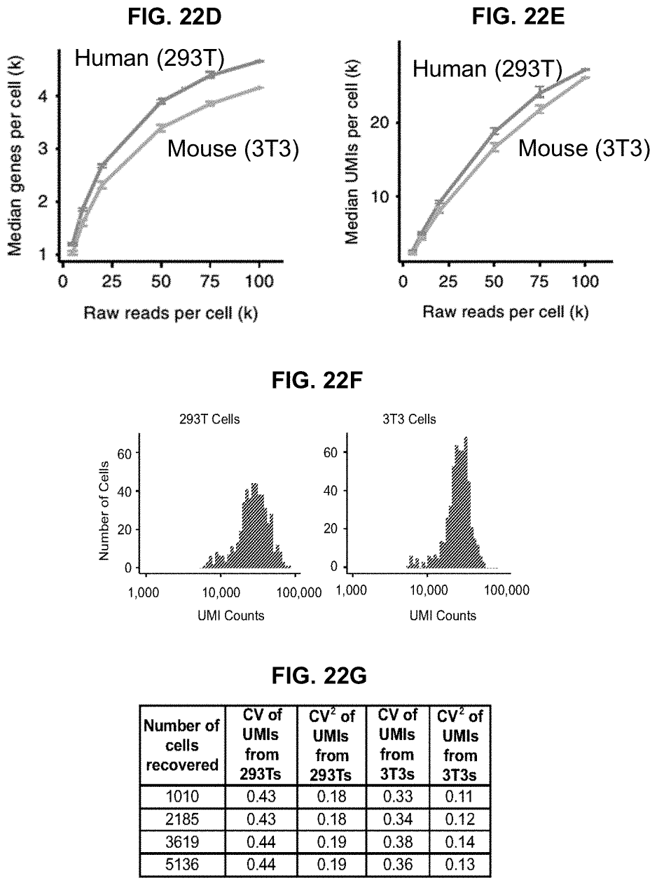

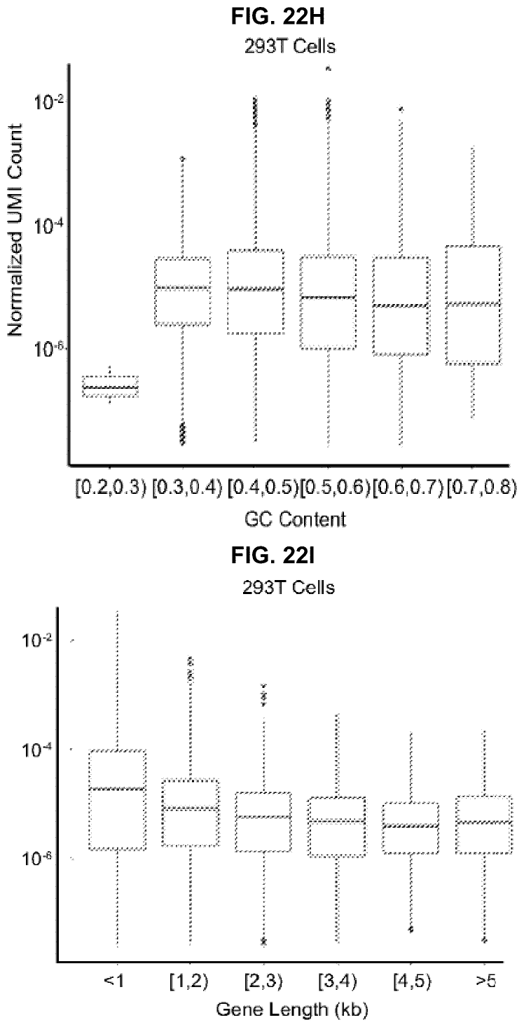

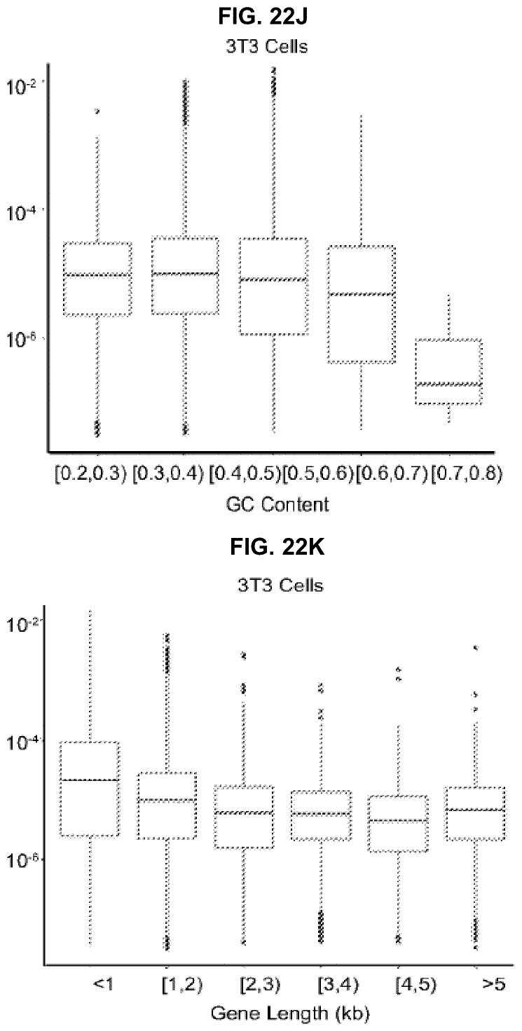

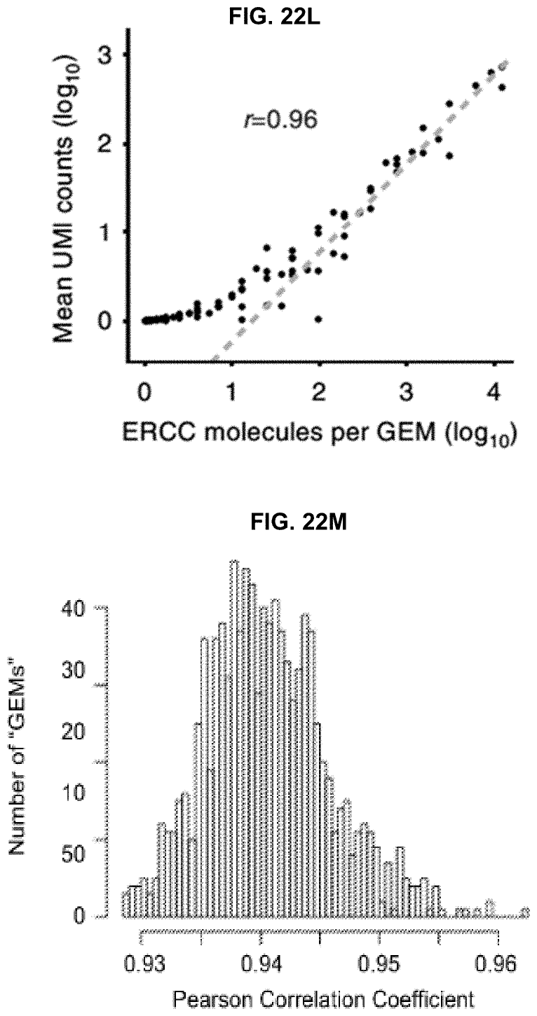

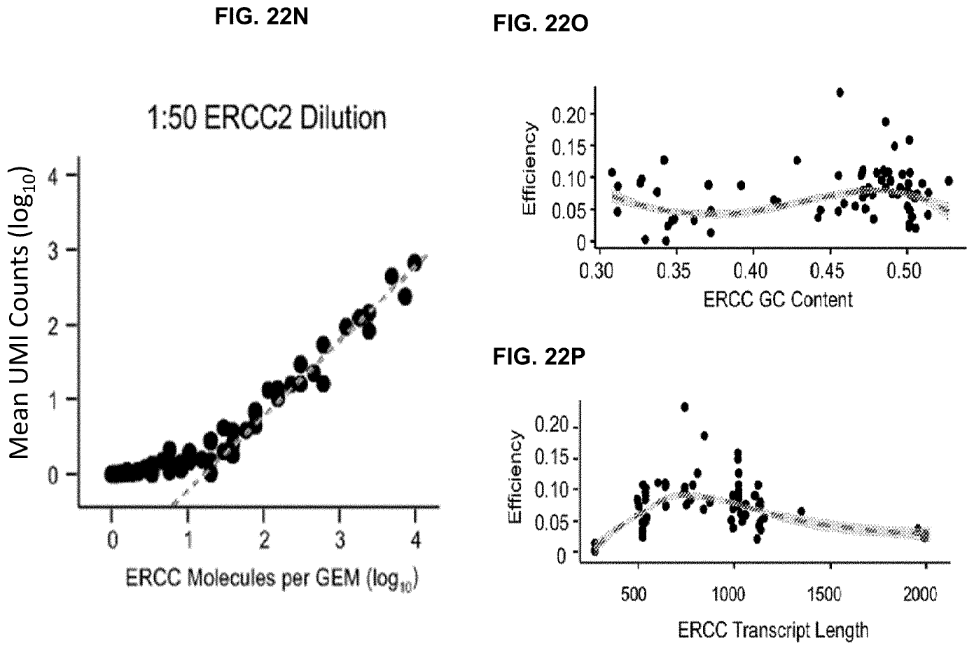

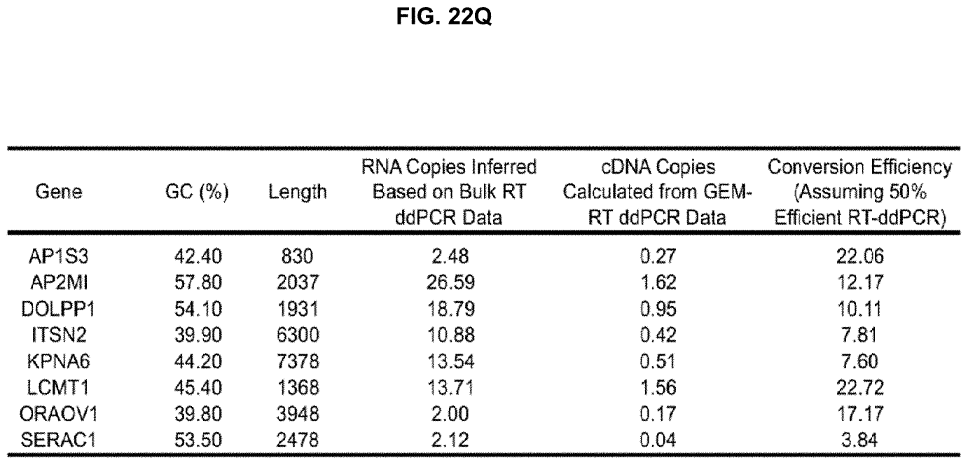

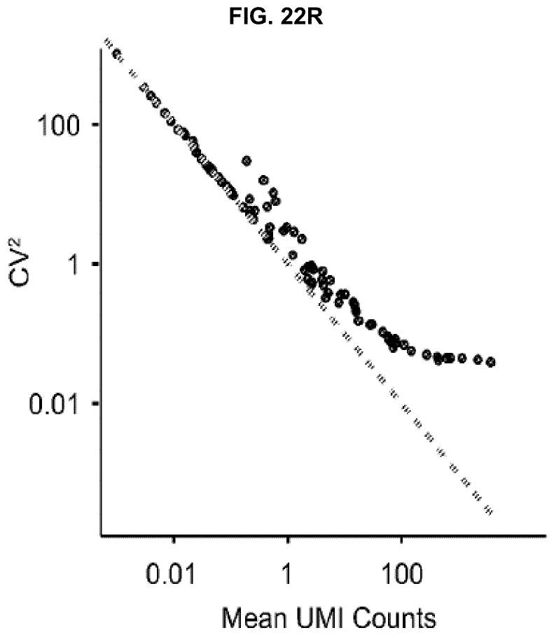

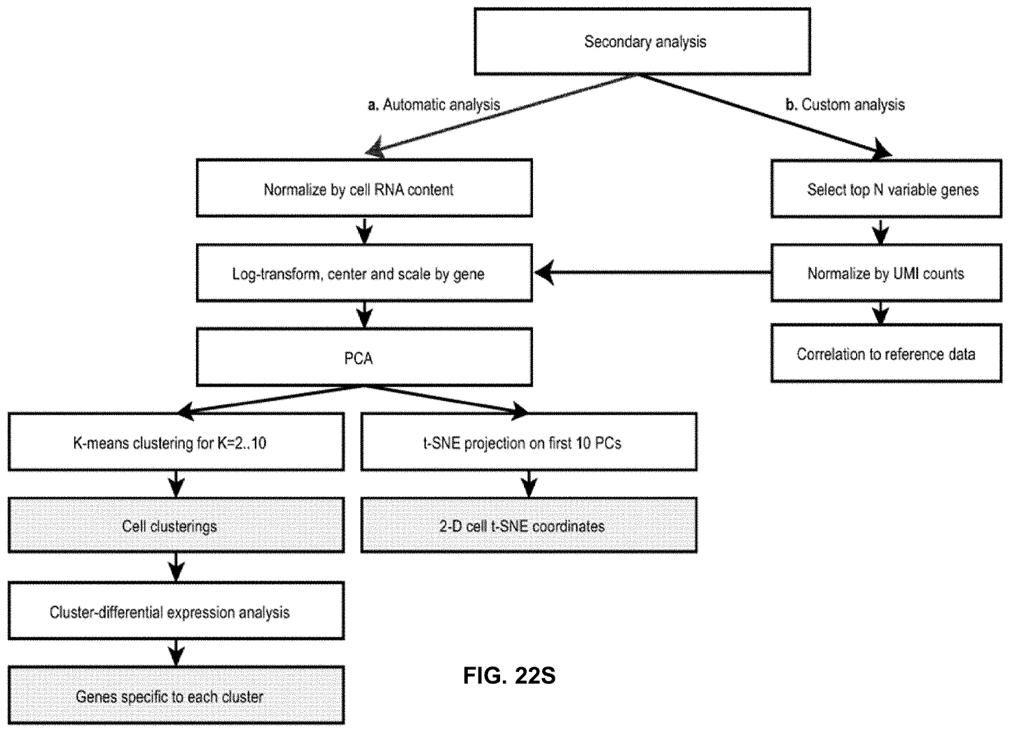

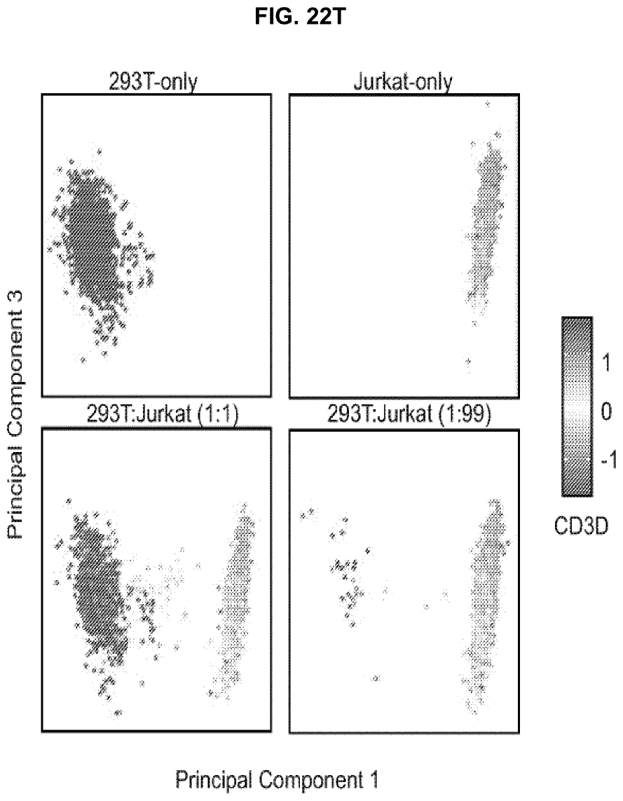

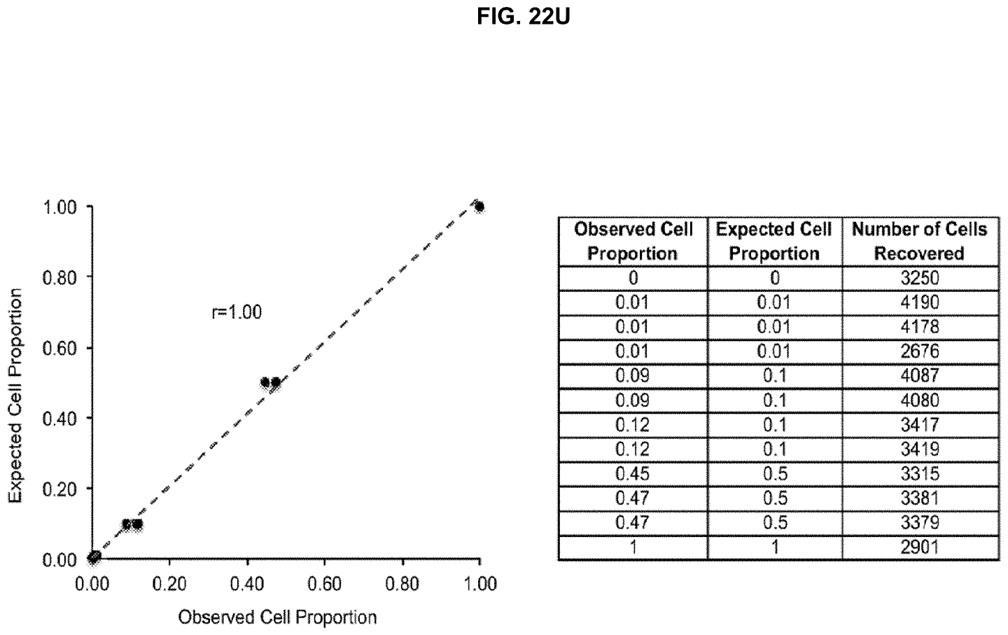

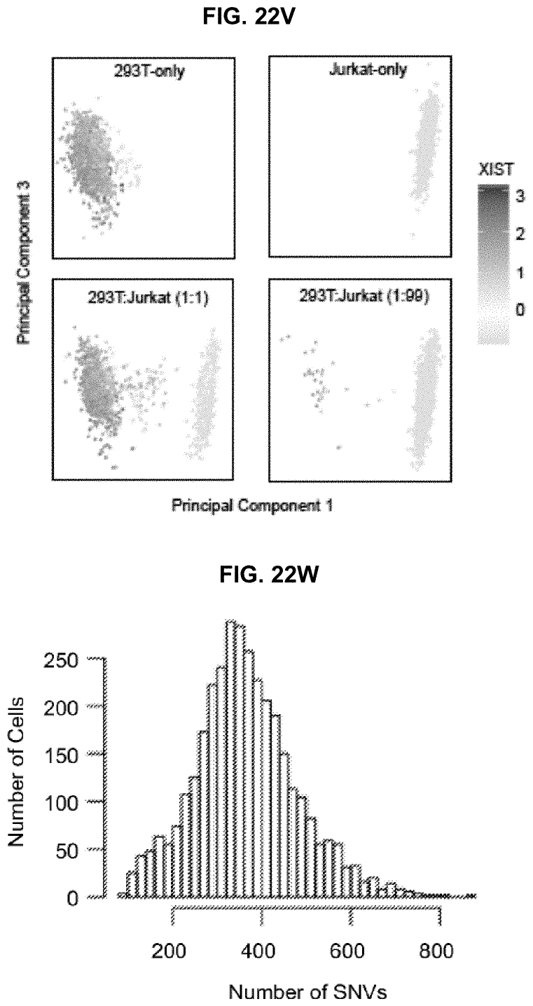

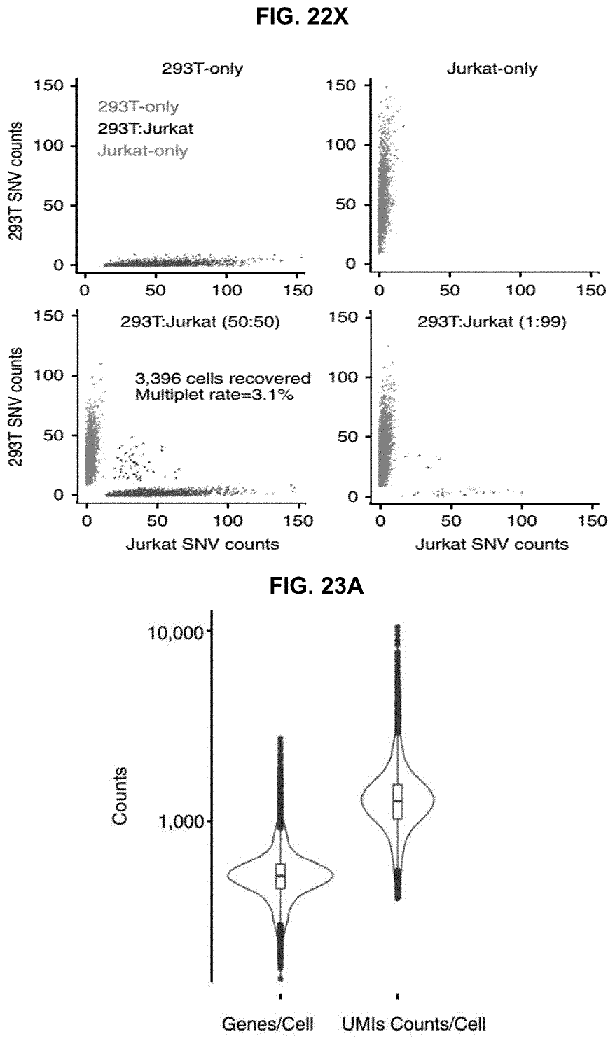

Based on the distribution of total UMI counts for each barcode, it was estimated that 1,012 GEMs contained cells, of which 482 and 538 contained reads that mapped primarily to the human and mouse transcriptome, respectively (and will be referred to in this example as human and mouse GEMs) (FIG. 22A). Greater than 83% of UMI counts were associated with cell barcodes, indicating low background of cell-free RNA. Eight cell-containing GEMs had a substantial fraction of human and mouse UMI counts (the UMI count is >=1% of each species' UMI count distribution), yielding an inferred multiplet rate, or rate of GEMs containing >1 cell, of 1.6% (FIG. 22A). A cell titration experiment across six different cell loads showed a linear relationship between the multiplet rate and the number of recovered cells ranging from 1,200 to 9,500 (FIG. 22B). The multiplet rate and trend are consistent with Poisson loading of cells, and were validated by independent imaging experiments (FIG. 22C). In addition, ˜50% cell capture rate was observed, which is the ratio of the number of cells detected by sequencing and the number of cells loaded. The capture rate was consistent across four types of cells with cell loading ranging from 1,000 to 23,000 (Table 1), an improvement over some scRNA-seq systems. Lastly, the mean fraction of UMI counts from the other species was approximately 0.9% in both human and mouse GEMs, indicating a low level of cross-talk between cell barcodes. Such performance metrics (e.g., low cross-talk between cell barcodes, low multiplet rate, and high cell capture rate), can improve analysis of samples with limited cell input and detection of rare cells.

At 100 k reads/cell, a median of ˜4,500 genes and ˜27,000 transcripts (UMI counts) in each human and mouse cell was detected, indicating comparable sensitivity to other droplet-based platforms (FIGS. 22D and 22E). UMI counts showed a standard deviation of ˜43% of the mean (CV) in human cells, and ˜33% of the mean in mouse cells, where the trend was consistent in four independent human and mouse mixture experiments (FIGS. 22F and 22G). Genes of different GC composition and length show similar UMI count distributions, suggesting low transcript bias (FIGS. 22H-22K).

The conversion rate of cDNA was also measured by loading External RNA Controls Consortium (ERCC) synthetic RNAs into GEMs in place of cells. The mean UMI counts from sequencing was highly correlated (r=0.96) with molecule counts calculated from the loading concentration of ERCC (FIGS. 22L and 22M). Furthermore, an efficiency of ˜6.7-8.1% from both ERCC RNA Spike-in Mix1 and Mix2 in different dilutions was inferred (FIG. 22N), with minimal evidence of GC bias, and limited bias for transcripts longer than 500 nt (FIGS. 22O and 22P). Additionally, the conversion rate of cell transcripts in Jurkat cells was estimated by ddPCR. The amount of cDNA of eight genes obtained from single cells after reverse transcription in GEMs was compared to the expected RNA inferred from bulk profiling. The conversion rates among genes were between 3.8% and 22.7%, which is consistent with ERCC data (FIG. 22Q).

The relative proportion of biological and technical variation was also estimated using the ERCC experiments. Since ERCCs are in solution, they are not expected to introduce biological variation, for example, biological variation related to differences in cell size, RNA content or transcriptional activity. Thus, technical variation is expected to be the primary source of variation. When the ERCCs are dilute (UMI counts are small) sampling noise can dominate; when the UMI counts increase, technical variations can become dominant (FIG. 22R). These variations include, but are not limited to, variation in droplet size, variation in concentration of RT reagents in the droplets, variation in the concentration of sample in the droplets, and variation in RT and/or PCR efficiency of the distinct gel bead barcode sequences. The squared coefficient of variation (CV2) was ˜7% among all the ERCC experiments. In comparison, CV2 in samples of mouse and human cells was ˜11-19% (FIG. 22G), suggesting that technical variance accounts for ˜50% of total variance.

B. Detection of Individual Populations in In-Vitro Mixed Samples.

The ability to accurately detect heterogeneous populations using a droplet based system described herein was tested by mixing two cell lines, 293T and Jurkat cells at different ratios (Table 1).

TABLE 1
Cell capture rate from 4 cell lines and 17 independent samples.

| | Number of | Number of Cells | Cell Capture |
|---|---|---|---|
| Cell Types | Cells Loaded | Recovered | Rate |
| HCC38 | 2,304 | 1,499 | 65% |
| HCC38 | 5,760 | 3,067 | 53% |
| HCC38 | 17,280 | 9,354 | 54% |
| HCC38 | 23,040 | 12,057 | 52% |
| 3T3 | 1,152 | 535 | 46% |
| 3T3 | 2,304 | 1,177 | 51% |
| 3T3 | 4,032 | 1,942 | 48% |
| 3T3 | 5,760 | 2,745 | 48% |
| 293T | 1,152 | 483 | 42% |
| 293T | 2,304 | 1,033 | 45% |
| 293T | 4,032 | 1,769 | 44% |
| 293T | 5,760 | 2,539 | 44% |
| PBMC | 2,304 | 1,001 | 43% |
| PBMC | 5,760 | 2,691 | 47% |
| PBMC | 11,520 | 5,952 | 52% |
| PBMC | 17,280 | 7,467 | 43% |
| PBMC | 23,040 | 10,123 | 44% |

After pooling all the samples, principal component analysis (PCA) was performed on UMI counts from all detected genes (FIG. 22S). In the sample where an equal number of 293T and Jurkat cells was mixed, principal component (PC) 1 separated cells into two clusters of equal size (FIGS. 22T and 22U). Based on expression of cell type specific markers, it was inferred that one cluster corresponded to Jurkat cells (preferentially expressing CD3D), and the other corresponded to 293T cells (preferentially expressing XIST, as 293T is a female cell line, and Jurkat is a male cell line) (FIGS. 22T and 22V). Points located between the two clusters are likely multiplets, as they expressed both CD3D and XIST (FIGS. 22T and 22V). In contrast, PCI did not separate cells into two clusters in the 293T-only and the Jurkat-only samples (FIG. 22T). Furthermore, in the sample with 1% 293T and 99% Jurkat cells, the numbers of cells in each of the two clusters were at the correct ratio (FIGS. 22T and 22U). A similar trend was observed for 12 independent samples where 293T and Jurkat cells were mixed at 5 different proportions, demonstrating the system's ability to perform unbiased detection of rare single cells (FIG. 22U).

In addition to providing a digital transcript count, sequencing data produced in this example provided ˜250 nt sequence for each cDNA that could be used for Single Nucleotide Variant (SNV) detection. On average, there were ˜350 SNVs detected in each 293T or Jurkat cell (FIG. 22W and Table 2).

TABLE 2
Total number of filtered SNVs and
median number of filtered SNV/cell.

| | | Median # of |
|---|---|---|
| | Total # of | Filtered |
| | Filtered | SNVs |
| | SNVs | detected |
| Samples | detected | per cell |
| 293T Cells | 19,595 | 321 |
| Jurkat Cells | 22,171 | 387 |
| 50%:50% Jurkat:293T Cell Mixture | 26,108 | 368 |
| 99%:1% Jurkat:293T Cell Mixture | 27,950 | 416 |
| Frozen PBMCs From Donor B | 14,157 | 55 |
| Frozen PBMCs From Donor C | 16,293 | 49 |
| 50%:50% Donor B:Donor C PBMC Mixture | 14,868 | 47 |
| 90%:10% Donor B:Donor C PBMC Mixture | 12,348 | 49 |
| 99%:1% Donor B:Donor C PBMC Mixture | 14,165 | 55 |
| AML027 Pre-transplant BMMCs | 8,900 | 37 |
| AML027 Post-transplant BMMCs | 12,374 | 80 |
| AML035 Pre-transplant BMMCs | 9,342 | 61 |
| AML035 Post-transplant BMMCs | 4,510 | 37 |

To determine whether or not the SNVs could be used to independently to distinguish cells in the mixture, a set of high quality SNVs that were only observed in 293T or Jurkat cells, but not both, were selected. Cells from the mixed samples were then scored based on the number of 293T or Jurkat-enriched SNVs. In the 1:1 mixed sample, ˜45% of 293T cells primarily (96%) harbored 293T-enriched SNVs, whereas ˜50% of Jurkat cells primarily (94%) harbored Jurkat-enriched SNVs (FIG. 22X). Jurkat and 293T cells inferred from marker-based analysis were 99% consistent with SNV-based assignment. A multiplet rate of ˜3% was observed, accounting for multiplets from Jurkats:293Ts as well as Jurkats:Jurkats and 293Ts:293Ts. The multiplet rate is consistent with that predicted from human and mouse mixing experiment, when ˜3000 cells were recovered (FIG. 22B). These results demonstrate that SNVs detected from scRNA-seq data can be used to classify individual cells.

C. Subpopulation Discovery from a Large Immune Population.

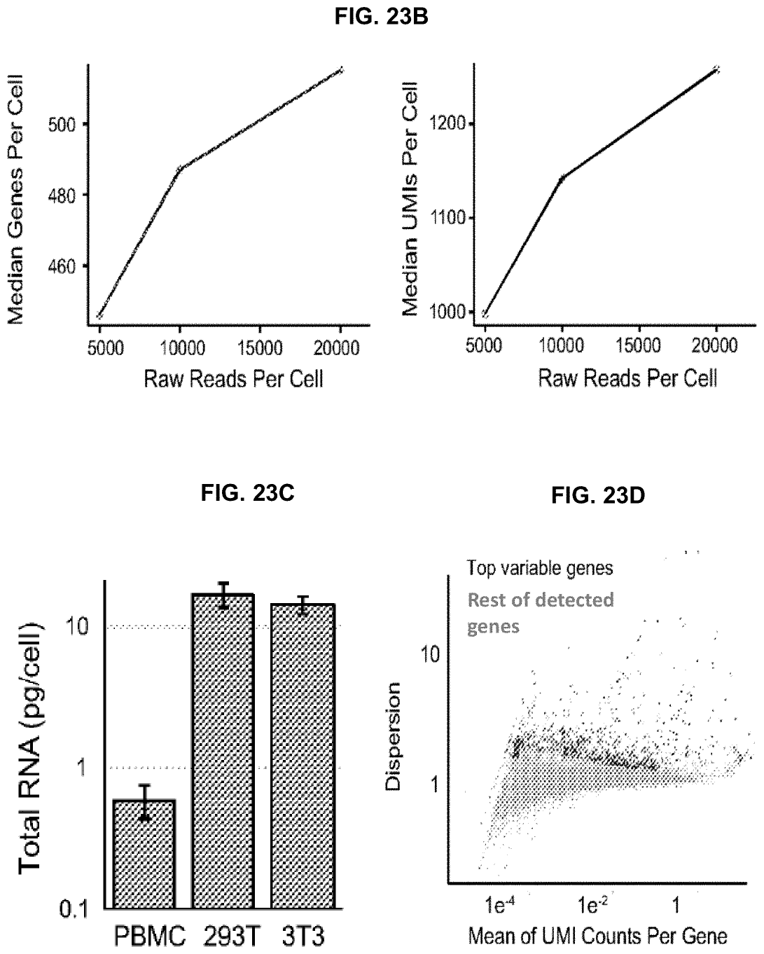

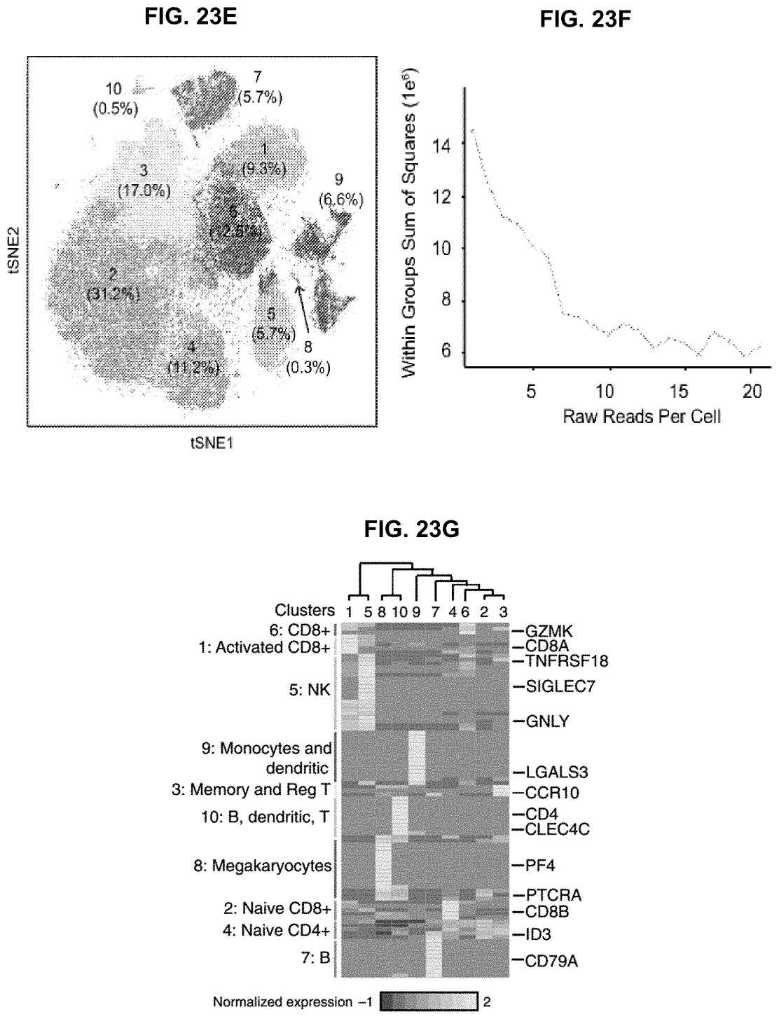

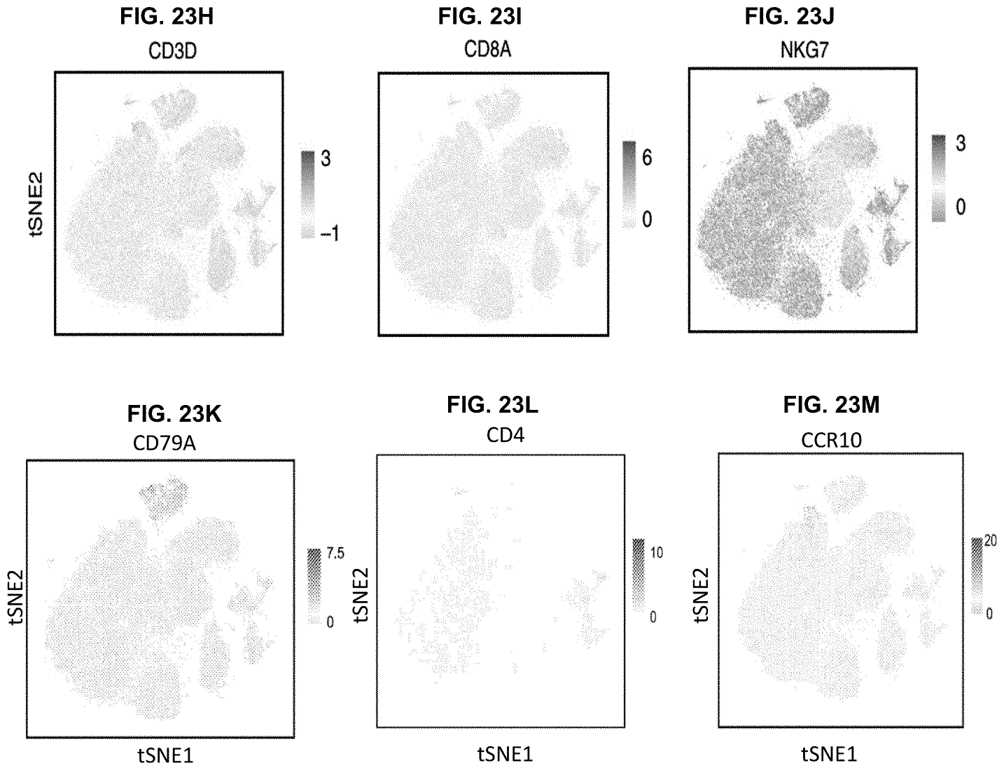

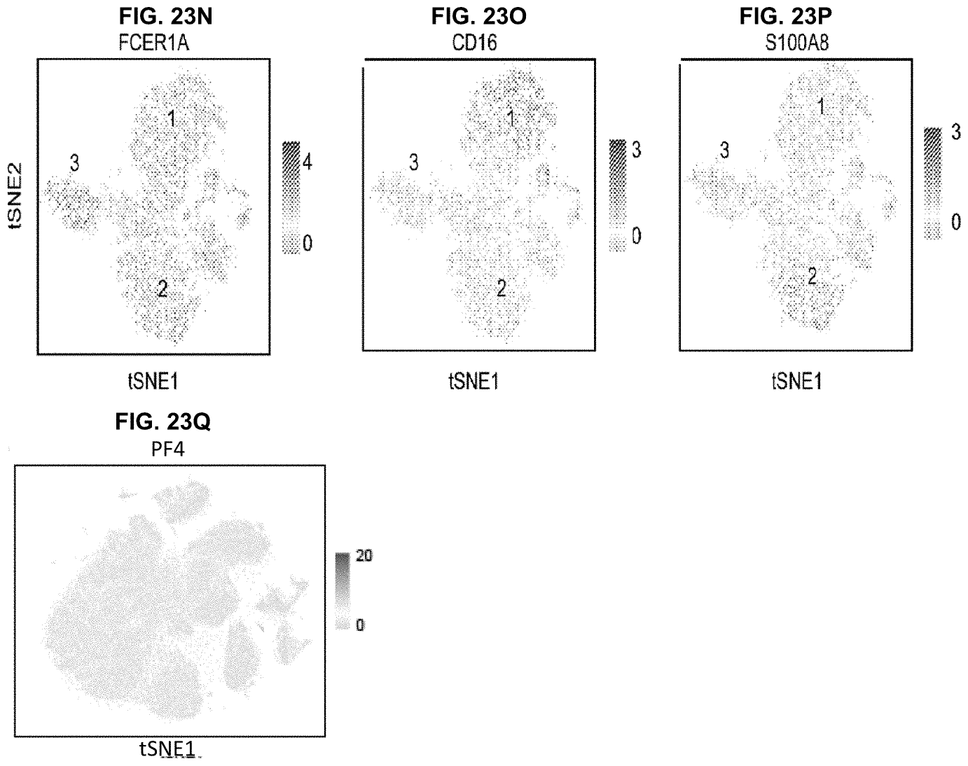

The methods and systems described herein for single cell analysis can also be used for scRNA-seq of primary cells. To study immune populations within peripheral blood mononuclear cells (PBMCs), fresh PBMCs from a healthy donor (Donor A) were obtained. Approximately 8 k-9 k cells were captured from each of 8 channels and pooled to obtain ˜68 k cells. Data from multiple sequencing runs were merged using a data analysis pipeline. At ˜20 k reads/cell, the median number of genes and UMI counts detected per cell were ˜525 and ˜1,300, respectively (FIG. 23A). The UMI count was roughly 10% of that from 293T and 3T3 samples at ˜20 k reads/cell, likely reflecting the differences in cells' RNA content (˜1 pg RNA/cell in PBMCs vs. ˜15 pg RNA/cell in 293T and 3T3 cells) (FIGS. 23B and 23C).

Clustering analysis was performed to examine cellular heterogeneity among PBMCs. PCA was applied on the top 1,000 variable genes ranked by their normalized dispersion, following a similar approach to Macosko et al. (Cell 2015, 161:1202-1214) (FIGS. 22S and 23D). K-means clustering on the first 50 PCs identified 10 distinct cell clusters, which were visualized in two dimensional projection of t-Distributed Stochastic Neighbor Embedding (tSNE) (FIGS. 23E and 23F). To identify cluster-specific genes, the expression difference of each gene between that cluster and average of the rest of clusters was calculated. Examination of the top cluster-specific genes revealed major subtypes of PBMCs at expected ratios: >80% T cells (enrichment of CD3D, part of the T cell receptor complex, in clusters 1-3, and 6), ˜6% NK cells (enrichment of NKG7 in cluster 5), ˜6% B cells (enrichment of CD79A in cluster 7) and ˜7% myeloid cells (enrichment of S100A8 and S100A9 in cluster 9 (FIGS. 23E and 23G-23K)). Finer substructures were detected within the T cell cluster; clusters 1, 4 and 6 are CD8+ cytotoxic T cells, whereas clusters 2 and 3 are CD4+ T cells (FIGS. 23I and 23L). The enrichment of NKG7 on cluster 1 cells implies a cluster of activated cytotoxic T cells (FIG. 23J). Cells in Cluster 3 showed high expression of CCR10 and TNFRSF18, a marker for memory T cells, and a marker for regulatory T cells respectively, likely consisted of a mixture of memory and regulatory T cells (FIGS. 23G and 23M). The presence of ID3, which is important in maintaining a naïve T-cell state, suggests that cluster 2 represents naïve CD4 T cells whereas cluster 4 represents naïve CD8 T cells (FIG. 23G). To identify sub-populations within the myeloid population, k-means clustering was further applied on the first 50 PCs of cluster 9 cells. At least 3 populations were evident: dendritic cells (characterized by presence of FCER1A), CD16+ monocytes, and CD16-/low monocytes (FIGS. 23N-23P). Overall, the results of this example demonstrate that systems and methods disclosed herein for scRNA-seq can detect most major subpopulations expected to be present in a PBMC sample.

Analysis of the results also revealed some minor cell clusters such as cluster 8 (0.3%) and cluster 10 (0.5%) (FIG. 23E). Cluster 8 showed preferential expression of megakaryocyte markers, such as PF4, suggesting that it represents a cluster of megakaryocytes (FIGS. 23E, 23G and 23Q). Cells in cluster 10 express markers of B, T and dendritic cells, suggesting a likely cluster of multiplets (FIGS. 23E and 23G). The size of the cluster suggests the multiplets comprised mostly B:dendritic and B:T:dendritic cells. With ˜9 k cells recovered per channel, it was expected that the multiplet rate would be ˜9% and the majority of multiplets would only contain T cells. More sophisticated methods may be required to detect multiplets from identical or highly similar cell types.

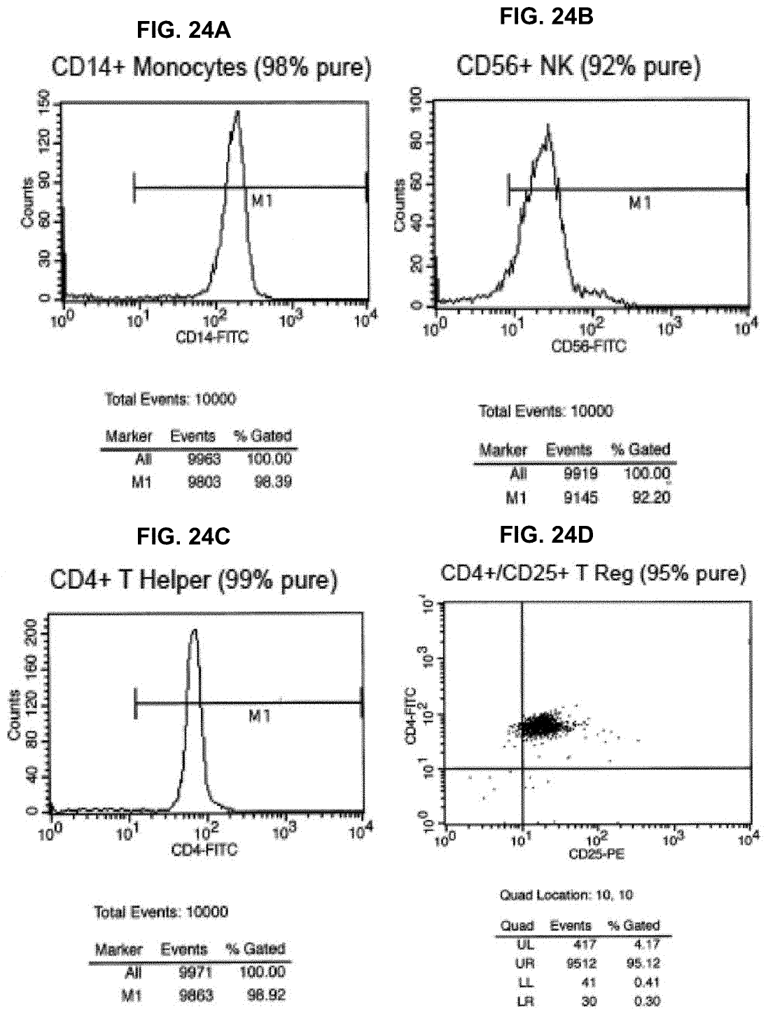

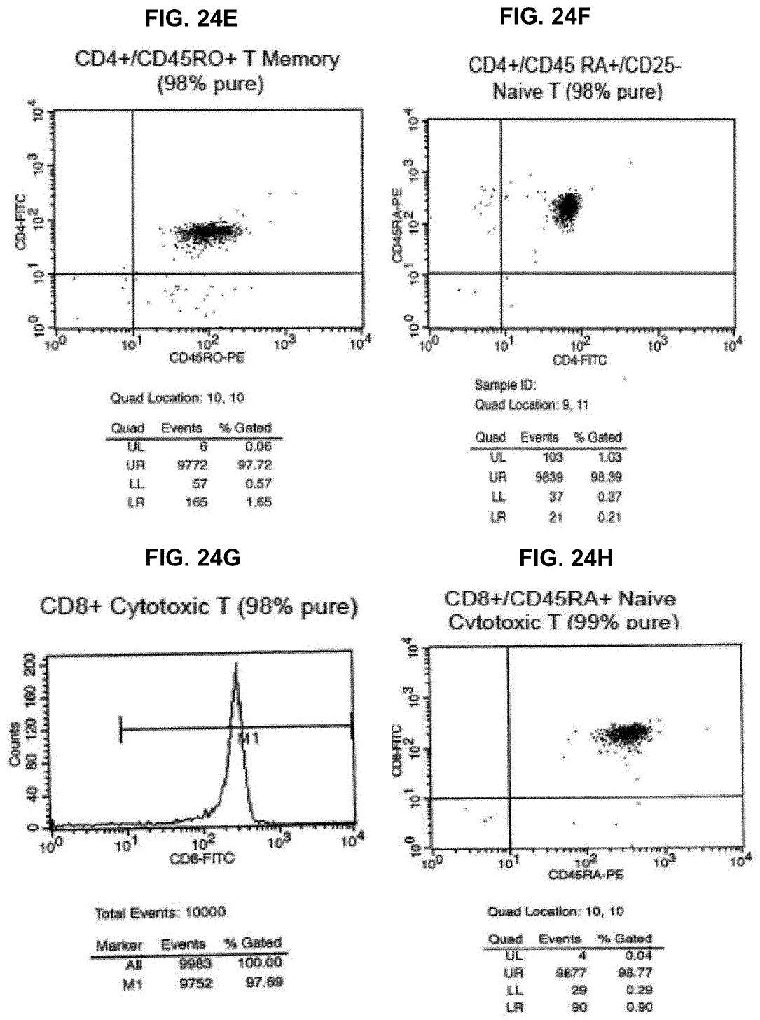

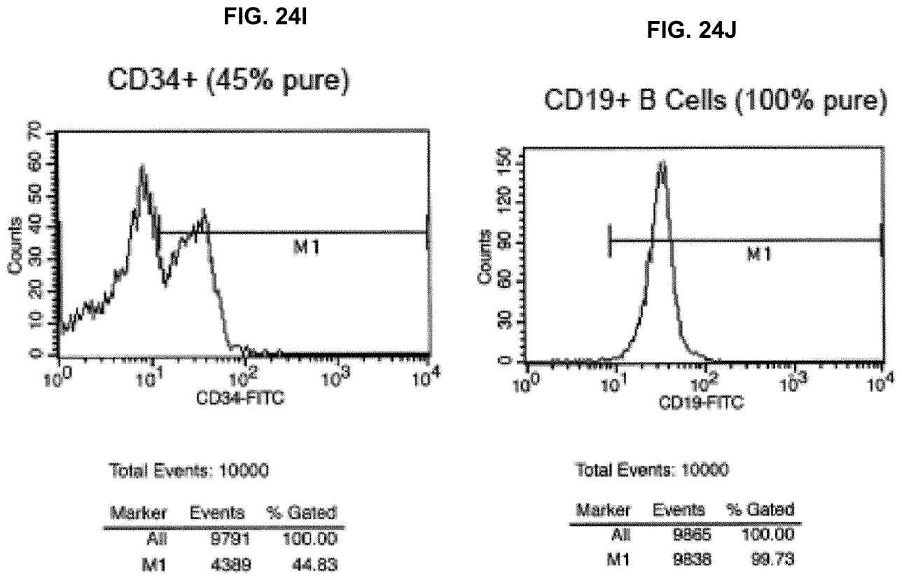

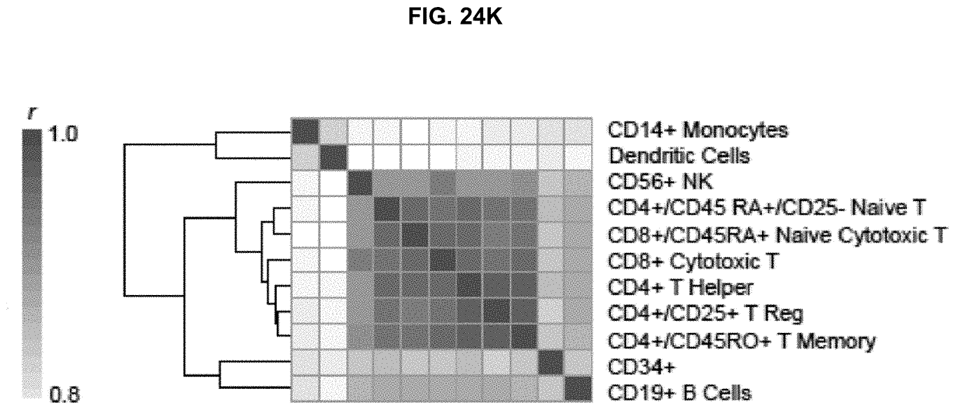

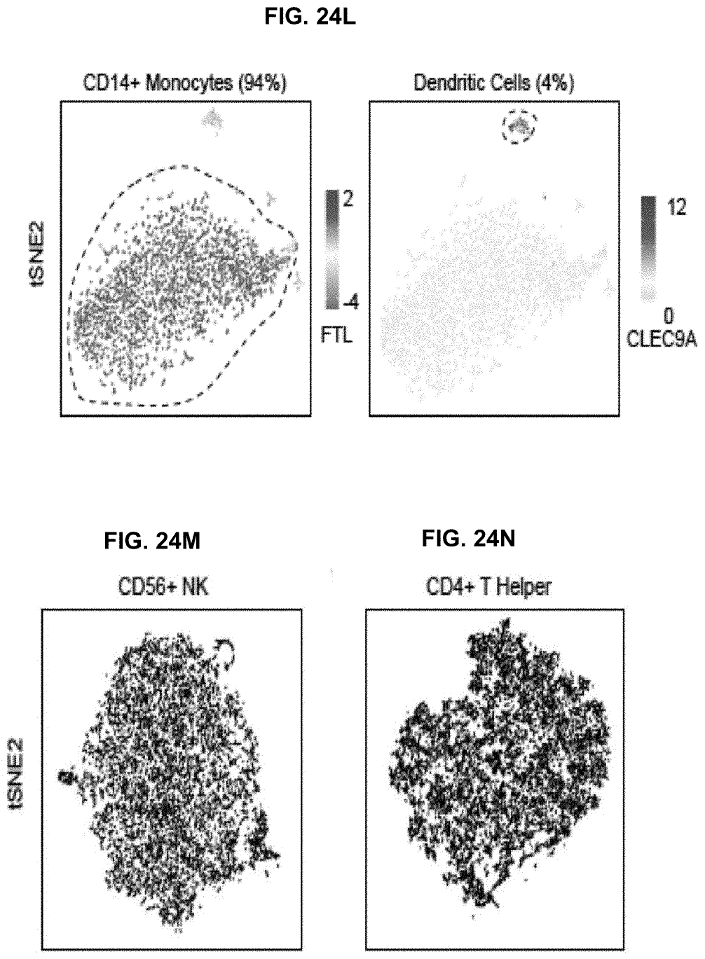

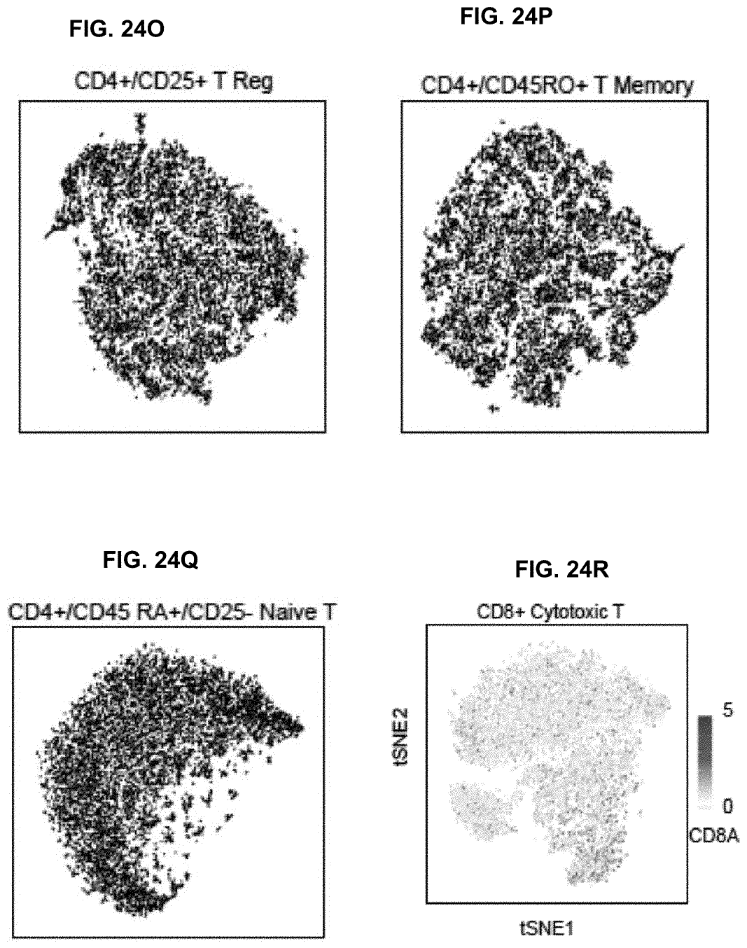

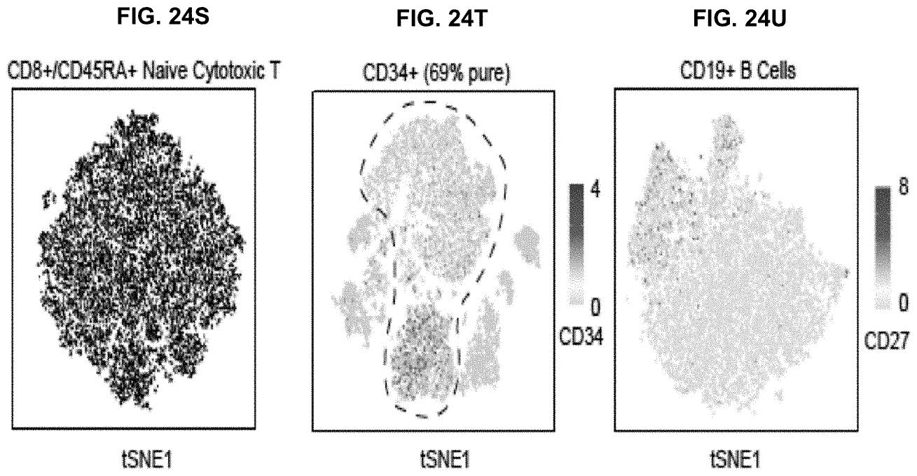

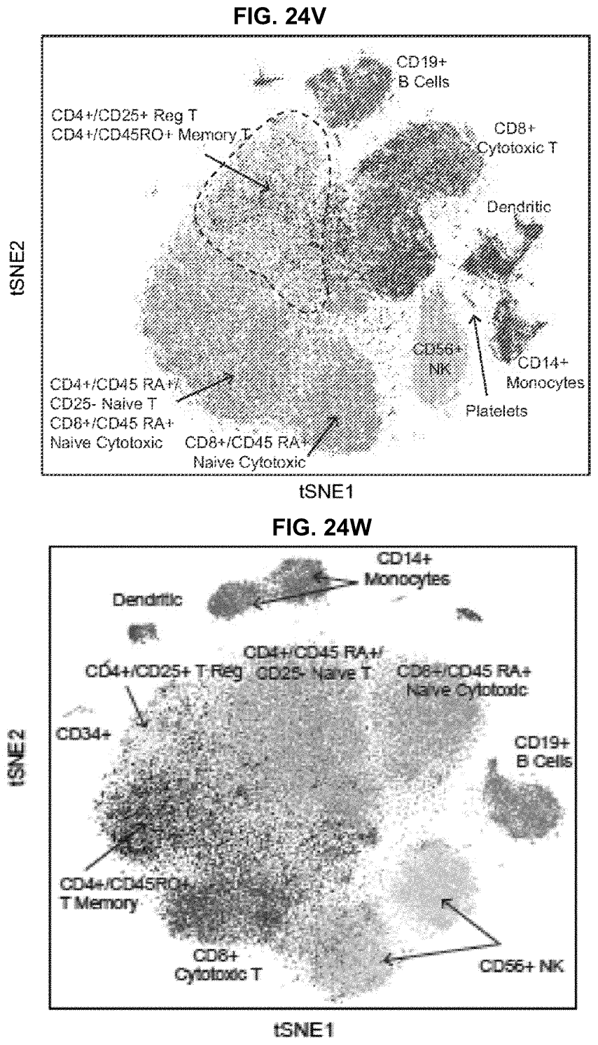

To further characterize the heterogeneity among 68 k PBMCs, reference transcriptome profiles were generated through scRNA-seq of 10 bead-enriched subpopulations of PBMCs from Donor A (FIGS. 24A-24U and Table 3).

TABLE 3
Bead-purification strategy of bead enriched PBMCs from Donor A.

| Cell types | Catalog numbers | Isolation methods |
|---|---|---|
| CD34+ cells | C-PB116-0.2M | Isolation kit from Milteny 130-046-701 |
| CD14+ Monocytes | C-PB114-10M7 | Negative selection using Stemcell 19059 |
| CD19+ B cells | C-PB106-10M7 | Negative selection from Stemcell 19054 |
| CD56+ NK cells | C-PB118-5M6 | Negative selection from Stemcell 19055 |
| CD8+ Cytotoxic T cells | C-PB105-10M | Negative selection from Stemcell 19053 |
| CD8+/CD45RA+ | C-PB125-5M3 | Negative selection from Stemcell 19058 |
| Naīve Cytotoxic T cells | | |
| CD4+/CD45RO+ | C-PB124-5M3 | Negative selection from Stemcell 19157 |
| Memory T cells | | |
| CD4+/CD45RA+/ | C-PB123-5M | Negative selection from Stemcell 19155 |
| CD25− Naīve T cells | | |
| CD4+/CD25+ | C-PB122-2M4 | Isolation kit from Stemcell 19052 to |
| Regulatory T cells | | isolate CD4, then isolate CD25 with |
| | | Miltenyi 130-092-983 |
| CD4+ Helper T | C-PB103-20M | Negative selection using Stemcell 19052 |

Clustering analysis revealed a lack of sub-structure in most samples, consistent with them being homogenous populations, and in agreement with FACS analysis (FIGS. 24A-24U). However, substructures were observed in CD34+ and CD14+ monocyte samples (FIGS. 24L and 24T). In the CD34+ sample, ˜70% cell clusters show expression of CD34 (FIG. 24T). In the CD14+ sample, the minor population showed marker expression for dendritic cells (e.g. CLEC9A), providing another reference transcriptome to classify the 68 k PBMCs (FIG. 24L). This result also demonstrates the power of scRNA-seq in selecting appropriate cells for further analysis.

The 68 k PBMCs were classified based on their best match to the average expression profile of 11 reference transcriptomes (FIG. 24V). Cell classification was largely consistent with previously described marker-based classification except that the boundaries among some of the T cell sub-populations were blurred. Namely, part of the inferred CD4+ naïve T population was classified as CD8+ T cells. The 68 k PBMC data was also clustered with Seurat. While it was able to distinguish inferred CD4+ naïve from inferred CD8+ naïve T cells, it was not able to cleanly separate out inferred activated cytotoxic T cells from inferred NK cells (FIG. 24W). Such populations have overlapping functions, making separation at the transcriptome level particularly difficult, if not unexpected. However, the complementary results suggest that more sophisticated clustering and classification methods can help address these challenges.

D. Single Cell RNA Profiling of Cryopreserved PBMCs.

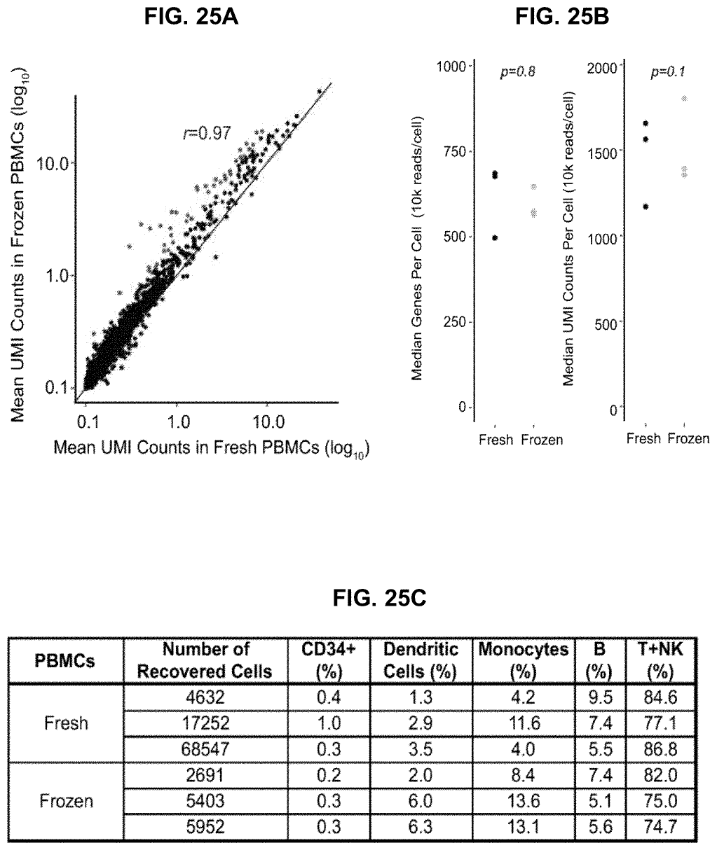

In order to analyze repository specimens for clinical research with the methods and systems disclosed herein, samples comprising cryopreserved cells were tested. The remaining fresh PBMCs from Donor A were frozen. Then, a scRNA-seq library was made from gently thawed cells a week later where ˜3 k cells were recovered. The two datasets (fresh and frozen) showed a high similarity between their average gene expression (r=0.97, FIGS. 25A-25C). Approximately 80 genes showed 2-fold upregulation in the frozen sample, with ˜50% being ribosomal protein genes, and the rest not enriched in any pathways (Table 4).

TABLE 4
List of genes that show ~2-fold upregulation
in scRNA-seq data of frozen PBMCs from Donor A.

| | Mean UMI | Mean UMI | Log2 Fold |
|---|---|---|---|
| | Counts (Frozen | Counts (Fresh | Change (Frozen |
| Gene ID | PBMCs) | PBMCs) | vs. Fresh |
| S100A1 | 1.16 | 0.45 | 1.36 |
| S100A9 | 2.82 | 0.37 | 2.92 |
| S100A8 | 1.81 | 0.28 | 2.67 |
| S100A6 | 3.14 | 1.39 | 1.17 |
| RPS27 | 14.23 | 6.65 | 1.10 |
| FCER1G | 1.10 | 0.48 | 1.21 |
| OST4 | 1.11 | 0.55 | 1.01 |
| RPL31 | 11.45 | 5.12 | 1.16 |
| RPL37A | 6.08 | 1.61 | 1.91 |
| RPL35A | 9.36 | 4.41 | 1.08 |
| RPL37 | 5.72 | 1.65 | 1.79 |
| COX7C | 1.63 | 0.68 | 1.26 |
| CD14 | 0.31 | 0.12 | 1.31 |
| LST1 | 0.93 | 0.46 | 1.01 |
| AIH1 | 1.16 | 0.55 | 1.07 |
| RPS10 | 3.40 | 1.31 | 1.38 |
| RPS12 | 18.94 | 8.43 | 1.17 |
| TOMM7 | 2.25 | 0.81 | 1.48 |
| TMFM176B | 0.32 | 0.16 | 1.04 |
| RPL36A | 2.59 | 0.90 | 1.52 |
| RPS20 | 7.06 | 3.29 | 1.10 |
| RPL30 | 10.40 | 4.28 | 1.28 |
| RPL35 | 8.38 | 3.64 | 1.20 |
| FCN1 | 0.69 | 0.22 | 1.63 |
| RPS24 | 6.26 | 2.42 | 1.37 |
| RPLP2 | 18.52 | 7.02 | 1.39 |
| MS4A6A | 0.25 | 0.12 | 1.03 |
| FAU | 7.90 | 3.65 | 1.11 |
| C12orf57 | 0.81 | 0.38 | 1.09 |
| RPS2G | 4.06 | 1.75 | 1.21 |
| LYZ | 2.61 | 0.52 | 2.33 |
| TPT1 | 12.96 | 5.05 | 1.36 |
| RPS29 | 2.76 | 0.73 | 1.92 |
| RPLP1 | 16.44 | 8.12 | 1.02 |
| TCEB2 | 0.80 | 0.40 | 1.02 |
| RPS15A | 13.23 | 5.94 | 1.16 |
| RPL23 | 3.00 | 1.23 | 1.29 |
| RPL27 | 7.04 | 2.51 | 1.49 |
| RPL38 | 3.57 | 0.96 | 1.90 |
| ZFAS1 | 1.06 | 0.51 | 1.07 |
| ATP5E | 1.94 | 0.86 | 1.17 |
| RPS21 | 3.60 | 0.87 | 2.05 |
| RPL36 | 6.69 | 2.82 | 1.25 |
| RPS28 | 6.10 | 2.04 | 1.58 |
| UBL5 | 0.73 | 0.36 | 1.01 |
| UBA52 | 7.67 | 3.18 | 1.27 |
| COX6B1 | 1.09 | 0.54 | 1.01 |
| HCS1 | 1.67 | 0.76 | 1.14 |
| TYROBP | 1.84 | 0.68 | 1.44 |
| RPS16 | 11.15 | 4.87 | 1.20 |
| RPS11 | 5.46 | 2.17 | 1.33 |
| RPL28 | 14.78 | 5.61 | 1.40 |
| LGALS1 | 1.27 | 0.58 | 1.14 |
| RP11-763B22.6 | 3.98 | 1.83 | 1.12 |
| RP11-403I13.5 | 3.39 | 1.46 | 1.21 |
| FCGR1C | 1.87 | 0.90 | 1.06 |

In addition, the number of genes and UMI counts detected from fresh and frozen PBMCs was very similar (p=0.8 and 0.1, respectively), suggesting that the conversion efficiency of the system is not compromised when profiling frozen cells (FIG. 25B). Furthermore, subpopulations were detected from frozen PBMCs at a similar proportion to that of fresh PBMCs, demonstrating the applicability of the methods and systems disclosed herein on cryopreserved samples (FIG. 25C).

E. Genotype-Based Method to Delineate Individual Populations from a Mixed Sample.

Next, the methods and systems disclosed herein were applied to study host and donor cell chimerism in an allogeneic hematopoietic stem cell transplant (HSCT) setting. To monitor treatment response and disease recurrence, the amount of host and donor chimerism has been measured by a panel of SNVs, and population changes examined by FACS. However, pre- and post-HSCT samples have shifting proportions of host and donor cells, each with dynamic changes in their cellular composition, making it challenging to separate and compare these sub-populations. Using the systems and methods disclosed herein, both immune cell subtypes and genotypes can be characterized by integrating scRNA-seq with de novo SNV calling.

While previous studies have used existing SNVs from DNA sequencing or large scale copy number changes (CNV) in the transcriptome data to distinguish cells by genotype, it is challenging to apply these methods to transplant samples where donor and host genotype is not known a priori, and when donor and host are closely matched in genotype. In this example, a method to infer the relative presence of host and donor genotypes in a mixed population based on SNVs directly from the transcriptome data was developed. The method identifies SNVs and infers a genotype at each SNV. It then classifies cells based on their genotypes across all SNVs.

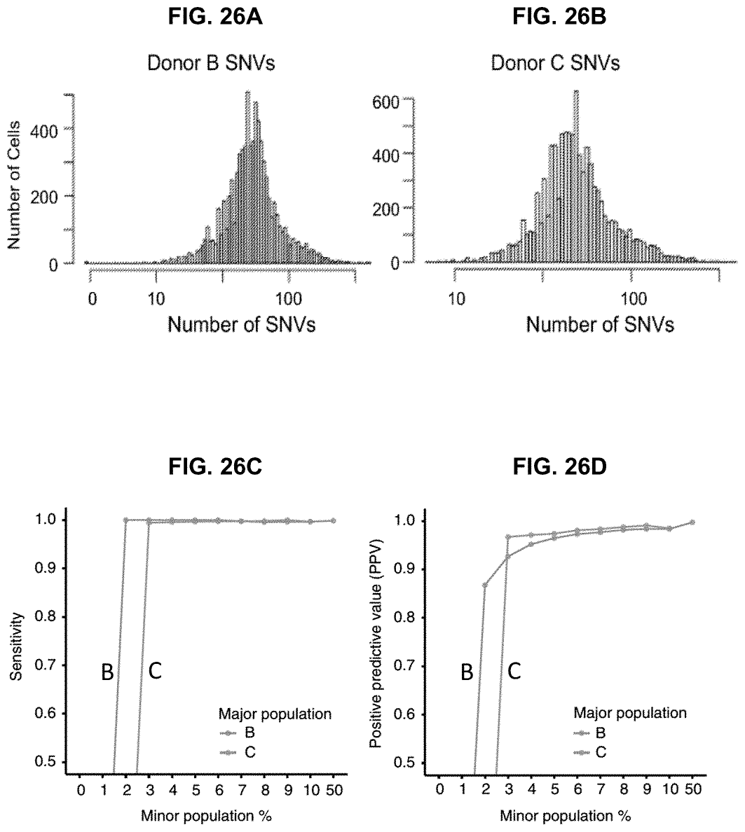

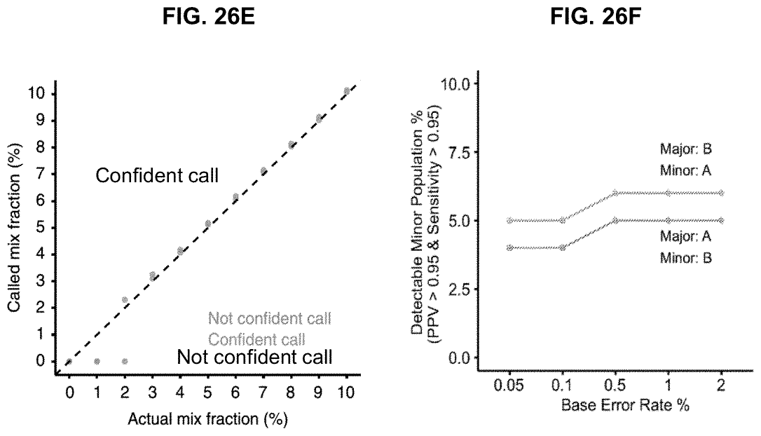

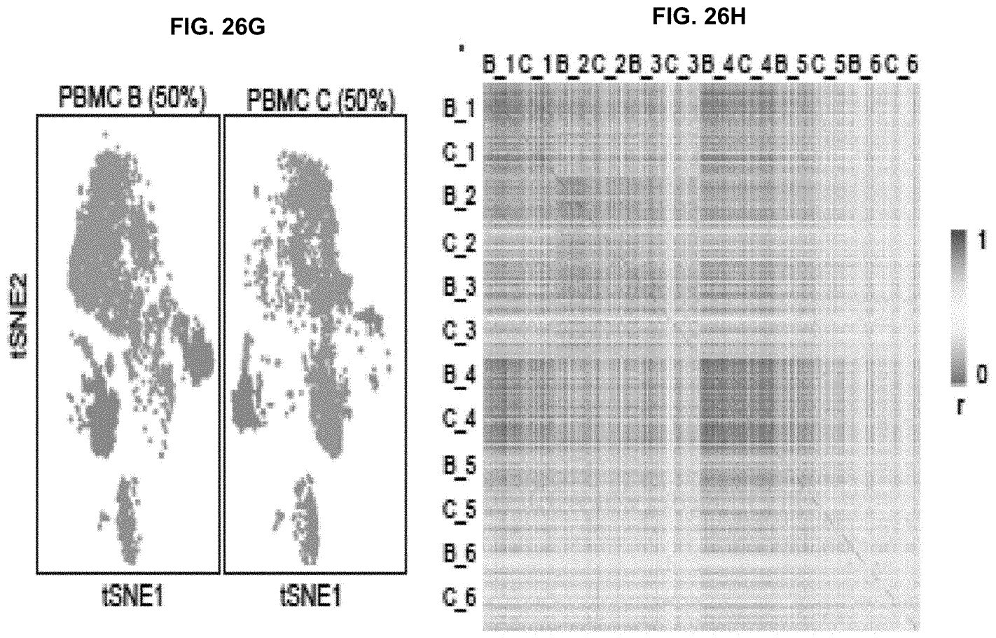

To evaluate the technical performance of the method, scRNA-seq libraries from PBMCs of 2 healthy donors B and C were generated, with ˜8 k cells captured for each sample. First, in silico mixing of PBMCs B and C at 12 mixing ratios ranging from 0 to 50% was performed. Only confidently mapped reads from samples B and C were used, and a total of 6000 cells were selected. There were ˜15 k reads/cell, with ˜50 filtered SNVs per cell (FIGS. 26A and 26B and Table 2).

Cells were then classified based on variants detected from the mixed transcriptome. Sensitivity and positive predictive value (PPV) were calculated by comparing predicted call of each cell against its true labeling. Using the systems and methods disclosed herein, minor genotypes as low as 3% were identified at >95% sensitivity and PPV (FIGS. 26C and 26D). A minor population could not be detected when the mixed ratio was below 3% (FIG. 26E). The accuracy can be affected by the number of observed SNVs per cell, which is dependent on cell types, diversity between subjects, and variant calling sensitivity. Nevertheless, the accuracy may not be very sensitive to base error rate or variant calling errors, as the method uses all instead of a small subset of SNVs (FIG. 26F).

The performance of the method of this example was further validated in experiments where PBMCs from Donors B and C were mixed at three ratios, 50:50, 90:10 and 99:1, prior to scRNA-seq. In the 1:1 mixture sample, cells from donors B and C were almost indistinguishable by RNA expression (FIGS. 26G and 26H). However, they can be separated by their genotype at the correct proportion (Table 5).

TABLE 5
Genotype comparison of predicted genotype
groups to purified populations.

| | | | | % Geno- | % Geno- |
|---|---|---|---|---|---|
| | | | | type | type |
| | Observed | Expected | | overlap | overlap |
| | % of | % of | Geno- | with | with |
| | minor | minor | type | Donor B | Donor C |
| | population | population | group | PBMCs | PBMCs |
| B only | 0 | 0 | 1 | 100 | 77 |
| C only | 0 | 0 | 1 | 77 | 100 |
| B:C = 50:50 | 43 | 50 | 1 | 63 | 94 |
| | | | 2 | 96 | 58 |
| B:C = 90:10 | 12 | 10 | 1 | 47 | 97 |
| | | | 2 | 82 | 74 |
| B:C = 99:1 | Not | 1 | 1 | 97 | 77 |
| | detected | | | | |

In addition, the genotype overlap between genotype group 1 and Donor C was 94%, whereas the overlap between genotype group 1 and Donor B was only 63%, both within the range of positive and negative controls, suggesting that group 1 comes from Donor C (Table 5). Similarly, genotype group 2 was inferred to be from Donor B (Table 5). The proportions of the minor genotype were accurately predicted at the 90:10 mixing ratio. Consistent with the in silico mixing results, the minor population could not be detected when B and C were mixed at 99:1 ratio (Table 5).

F. Single Cell Analysis of Transplant Bone Marrow Samples.

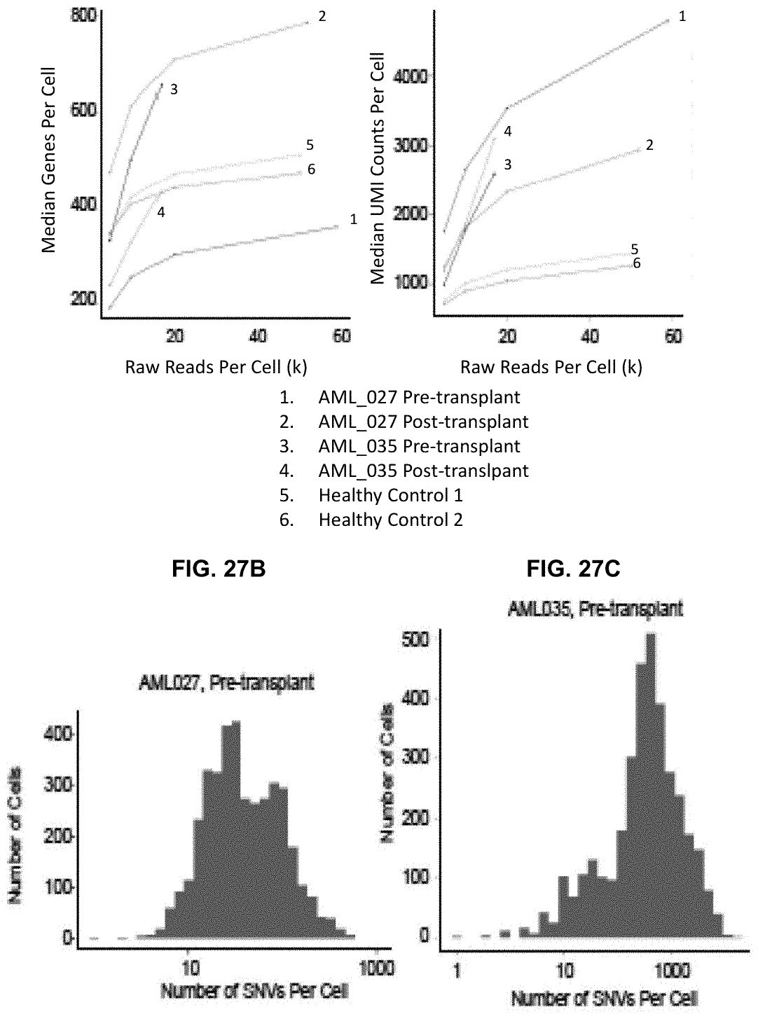

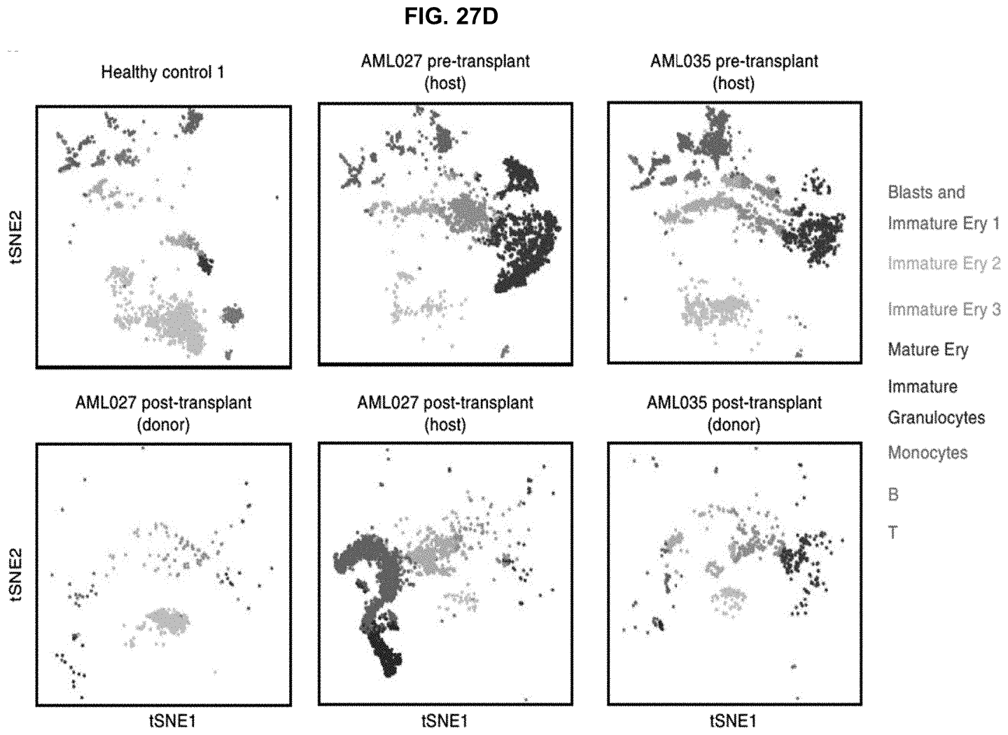

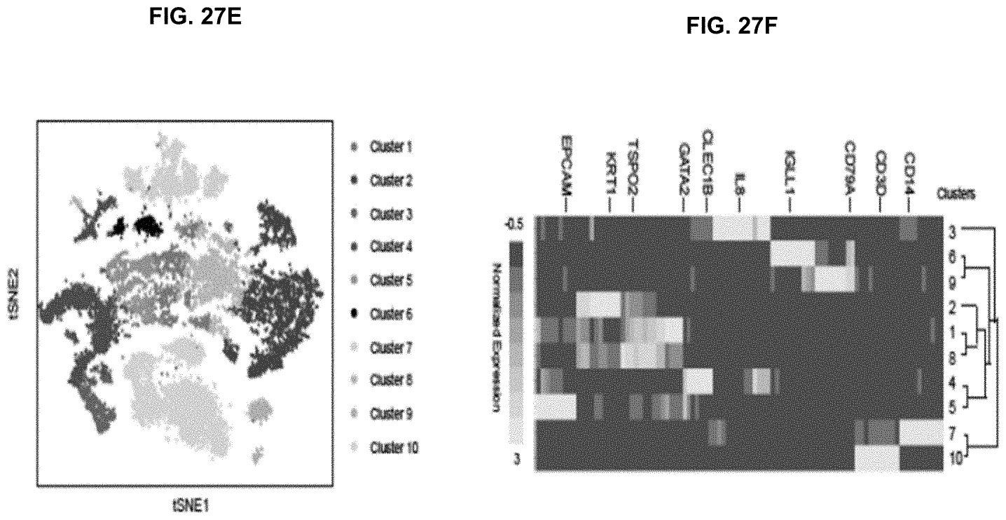

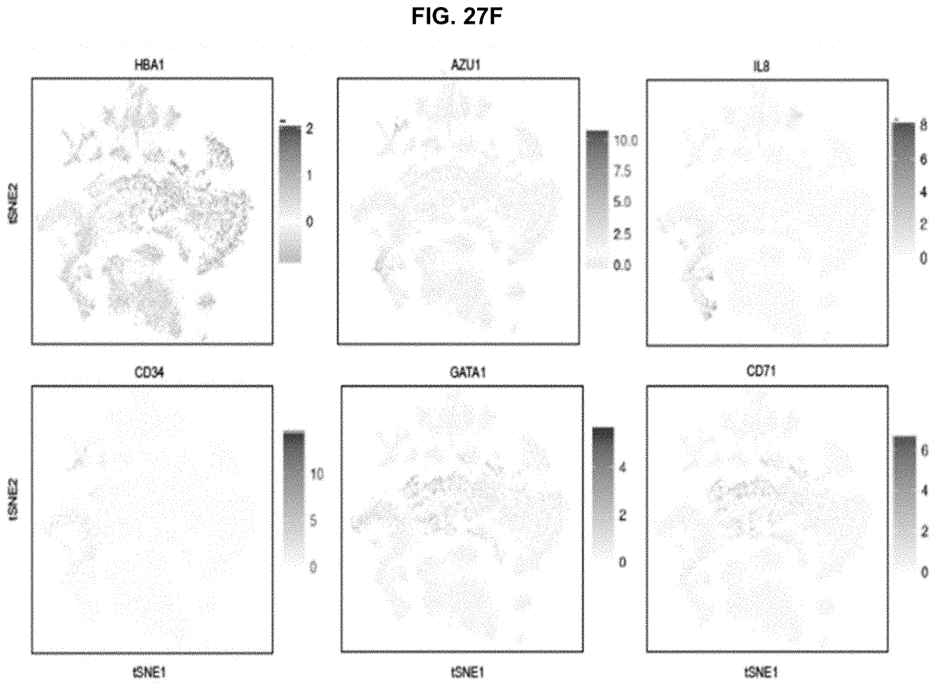

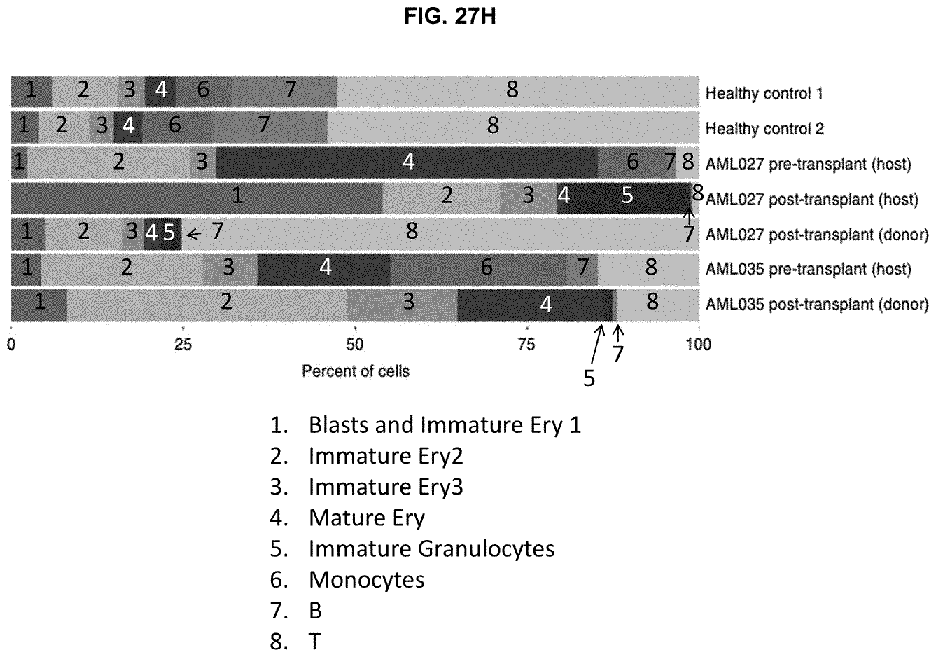

Single cell RNA-seq libraries were generated from cryopreserved bone marrow mononuclear cell (BMMC) samples of two patients before and after undergoing HSCT for acute myeloid leukemia (AML) (AML027 and AML035). Since HSCT samples are fragile, cells were carefully washed in PBS with FBS before loading them into chips. Relative to BMMCs from 2 healthy controls, 3-5 times as many median number of UMI counts per cell in AML samples at ˜15 k reads/cell were found, suggesting their vastly abnormal transcriptional programs (FIG. 27A). Approximately 35 and 60 SNVs/cell were detected from AML027 and AML035 pre-transplant samples respectively (FIGS. 27B and 27C). SNV analysis detected the presence of two genotypes in the post-transplant sample of AML027, one at 13.8%, and one at 86.2% (Table 6). As expected, there was no evidence of multiple genotype groups in the pre-transplant host sample. The major and minor inferred genotypes present were compared in the post-transplant sample to the genotype found in the host cells. The major inferred genotype in the post-transplant sample was 97% similar to that inferred from the host sample, while the minor inferred genotype was only 52% similar to that of the host sample (Table 6).

TABLE 6
Predicted genotype groups and their genotype
overlap with pre-transplant samples.

| | | | % of | |
|---|---|---|---|---|
| | | | Genotype | |
| | Geno- | % of | overlap with | |
| | type | Genotype | pre-transplant | Likely |
| Sample | group | group | sample (host) | identity |
| AML027 | 1 | 13.8 | 52 | Donor |
| post-transplant | 2 | 86.2 | 97 | Host |
| AML035 | 1 | 100 | 78 | Donor |
| post-transplant | | | | |

AML, acute myelod leukaemia.

The observed range of genotype overlap between the same individuals is ˜98%, indicating errors in the genotypes inferred from individual SNVs. However, 97% is within the observed range, and this results suggests that the post-transplant sample consists mainly (86.2%) of host cells. This observation is consistent with the clinical chimerism assay, which demonstrated only 12% donor in the post-transplant sample. In contrast, SNV analysis on the post-HSCT sample from AML035 did not detect the presence of 2 genotype groups. The sample only shared 78% similarity with AML035 host cells, suggesting that the post-HSCT sample was all donor-derived (Table 6). This finding was validated by the independent clinical chimerism assay.

SNV and scRNA-seq analyses enable subpopulation comparison between individuals within and across multiple samples. These analyses were applied on BMMC scRNA-seq data from healthy controls and AML patients, and a few subpopulation differences in AML patients after HSCT were observed. First, while T cells dominate the healthy BMMCs and donor cells of AML027 post-transplant sample as expected, erythroids constituted the largest population among AML samples (FIG. 27D). Different sets of progenitor and differentiation markers (e.g. CD34, GATA1, CD71 and HBA1) were detected among the erythroids, indicating populations at various stages of erythroid development (FIGS. 27E-27G). AML027 showed the highest level of erythroid cells (>80%, consist of mostly mature erythroids) before transplant, consistent with the erythroleukemia diagnosis of AML027 (FIG. 27H). In contrast, after transplant, AML027 showed the highest level of blast cells and immature erythroids (CD34+, GATA1+), consistent with the relapse diagnosis and return of the malignant host AML (FIG. 27H). These observations would have been difficult to make with FACS analysis, with limited number of markers for early erythroid lineages. Second, ˜20% cells in AML027 post-transplant sample show markers of immature granulocytes (AZU1, IL8, FIG. 27E-27H), which are absent in AML035, and generally low among AML patients. These cells lack marker expression for mature cells, suggesting the presence of residual precursor cells that may be part of the leukemic clone. Third, monocytes are abundant in both AML patients before transplant (10% and 25% in AML027 and AML035 respectively), but are not detectable after transplant (FIG. 27H). Monocytes have been previously identified in post-transplant samples, and the unexpected monocytopenia needs to be followed up with additional studies. Taken together, the analysis provided insights into the cellular composition and presence of residual disease in the bone marrows of HSCT recipients that was not available from routine clinical assays.

This example demonstrates use of the methods and systems disclosed herein for digital profiling of thousands to tens of thousands of cells per sample, specifically in profiling large immune systems, where substructures within 68 k PBMCs were studied. The ability to generate faithful scRNA-seq profiles from cryopreserved samples with high cell capture efficiency enables the application of scRNA-seq to clinical samples. scRNA-seq samples were successfully generated from fragile BMMCs of transplant samples, and the proportion of donor and host genotypes were correctly estimated. In addition, clustering analysis provided a richer understanding of the complex interplay between host and donor cells and of multiple lineages in the post-transplant setting. It provided insights into early erythroid lineage, and offered a much richer understanding of patients' disease progression that would have been limited with routine FACS analysis and clinical chimerism tests.

G. Methods

High Speed Imaging of Gel Beads and Cells in GEMs

A microscope (Nikon Ti-E, 10× objective) and a high speed video camera (frame rate=4000/s) was used to image every GEM as they were generated in the microfluidic chip. A custom image analysis software was used to detect the number of gel beads and cells in every GEM. The detection was based on the contrast between both the edge of a bead, a cell and the edge of a GEM against the adjacent liquid. To estimate the distribution of cells in GEMs, manual counting was used for ˜28 k frames of one video. The results indicate an approximate adherence to a Poisson distribution. However, the percentage of multiple cell encapsulations was 16% higher than the expected value, possibly due to sub-sampling error or to cell-cell interactions (some two-cell clumps were observed during the manual count).

Cell Lines and Transplant Patient Samples

Jurkat (ATCC TIB-152), 293T (ATCC CRL-11268) and 3T3 (ATCC CRL-1658) cells were acquired from ATCC and cultured according to ATCC guidelines. Fresh PBMCs, frozen PBMCs and BMMCs were purchased from ALLCELLS.

The Institutional Review Board at the Fred Hutchinson Cancer Research Center approved the study on transplant samples. The procedures followed were in accordance with the Helsinki Declaration of 1975 and the Common Rule. Samples were obtained after patients had provided written informed consent on molecular analyses. Patients with AML undergoing allogeneic hematopoietic stem cell transplant were identified at the Fred Hutchinson Cancer Research Center. The diagnosis of AML was established according to the revised criteria of the World Health Organization.

Bone marrow aspirates were obtained for standard clinical testing 20-30 days before transplant and serially post-transplant according to the treatment protocol. Bone marrow aspirate aliquots were processed within 2 hours of the draw. The BMMCs were isolated using centrifugation through a Ficoll gradient (Histopaque-1077, Sigma Life Science, St Louis, Mo.). The BMMCs were collected from the serum-Ficoll interface with a disposable Pasteur pipet and transferred to the 50 ml conical tube with 2% patient serum in 1×PBS. The BMMCs were counted using a hemacytometer and viability was assessed using Trypan Blue. The BMMCs were resuspended in 90% FBS, 10% DMSO freezing media and frozen using a Thermo Scientific Nalgene Mr. Frosty (Thermo Scientific) in a −80° C. freezer for 24 hours before transferred to liquid nitrogen for long-term storage.

Estimation of RNA Content Per Cell

The amount of RNA per cell type was determined by quantifying (Qubit, Invitrogen) RNA extracted (Maxwell RSC simplyRNA Cells Kit) from several different known number of cells.

Cell Preparation

Fresh cells were harvested, washed with 1×PBS and resuspended at 1×106 cells/ml in 1×PBS and 0.04% BSA. Fresh PBMCs were frozen at 10× by resuspending PBMCs in DMEM+20% FBS+10% DMSO, freezing to −80° C. in a CoolCell® FTS30 (BioCision), then placed in liquid nitrogen for storage.

Frozen cell vials from ALLCELLS and transplant studies were rapidly thawed in a 37° C. water bath for approximately 2 minutes. Vials were removed when a tiny ice crystal was left. Thawed PBMCs were washed twice in medium then resuspended in 1×PBS and 0.04% BSA at room temperature. Cells were centrifuged at 300 rcf for 5 min each time. Thawed BMMCs were washed and resuspended in 1×PBS and 20% FBS. The final concentration of thawed cells was 1×10⁶ cells/ml.

Sequencing Library Construction Using the GemCode Platform

Cellular suspensions were loaded on a GemCode Single Cell Instrument (10× Genomics, Pleasanton, Calif.) to generate single cell GEMs. Single cell RNA-Seq libraries were prepared using GemCode Single Cell 3′ Gel Bead (P/N 120217) and Library Kit (P/N 120218, 10× Genomics). GEM-RT was performed in a C1000 Touch™ Thermal cycler with 96-Deep Well Reaction Module (Bio-Rad P/N 1851197): 55° C. for 2 hours, 85° C. for 5 minutes; held at 4° C. After RT, GEMs were broken and the single strand cDNA was cleaned up with DynaBeads® MyOne™ Silane Beads (Thermo Fisher Scientific P/N 37002D) and SPRIselect Reagent Kit (0.6×SPRI, Beckman Coulter P/N B23318). cDNA was amplified using the C1000 Touch™ Thermal cycler with 96-Deep Well Reaction Module: 98° C. for 3 min; cycled 14×: 98° C. for 15 s, 67° C. for 20 s, and 72° C. for 1 min; 72° C. for 1 min; held at 4° C. Amplified cDNA product was cleaned up with the SPRIselect Reagent Kit (0.6×SPRI). The cDNA was subsequently sheared to ˜200 bp using a Covaris M220 system (Covaris P/N 500295). Indexed sequencing libraries were constructed using the reagents in the GemCode Single Cell 3′ Library Kit, following these steps: 1) end repair and A-tailing; 2) adapter ligation; 3) post-ligation cleanup with SPRIselect; 4) sample index PCR and cleanup. The barcode sequencing libraries were quantified by quantitative PCR (qPCR) (KAPA Biosystems Library Quantification Kit for Illumina platforms P/N KK4824). Sequencing libraries were loaded at 2.1 pM on an Illumina NextSeq500 with 2×75 paired-end kits using the following read length: 98 bp Read1, 14 bp 17 Index, 8 bp 15 Index and 10 bp Read2. Some earlier libraries were made with 5 nt UMI, and 5 bp Read2 was obtained instead.

ERCC Assay

ERCC synthetic spike-in RNAs (Thermo Fisher P/N 4456740) were diluted (1:10 or 1:50) and loaded into a GemCode Single Cell Instrument, replacing cells normally used to generate GEMs. Spike-in Mix1 and Mix2 were both tested. A slightly modified protocol was used as only a small fraction of GEMs were collected for RT and cDNA amplification. After the completion of GEM-RT, 1.25 pL of the emulsion was removed and added to a bi-phasic mixture of Recovery Agent (125 pL) (P/N 220016) and 25 mM Additive 1 (30 pL) (P/N 220074, 10× Genomics). The recovery agent was then removed and the remaining aqueous solution was cleaned up with the SPRIselect Reagent Kit (0.8×SPRI). cDNA was amplified using the C1000 Touch™ Thermal cycler with 96-Deep Well Reaction Module: 98° C. for 3 min; cycled 14×: 98° C. for 15 s, 67° C. for 20 s, and 72° C. for 1 min; 72° C. for 1 min; held at 4° C. Amplified cDNA product was cleaned up with the SPRIselect Reagent Kit (0.8×) cDNA was subsequently sheared to ˜200 bp using a Covaris M220 system to construct sample-indexed libraries with 10× Genomics adapters. Expected ERCC molecule counts were calculated based on the amount of ERCC molecules used and sample dilution factors. The counts were compared to detected molecule counts (UMI counts) to calculate conversion efficiency.

ddPCR Assay

Jurkat cells were used in ddPCR assays to estimate conversion efficiency as follows. 1) The amount of RNA per Jurkat cell was determined by quantifying (Qubit, Invitrogen) RNA extracted (Maxwell RNA Purification Kits) from several different known number of Jurkat cells. 2) Bulk RT-ddPCR (Bio-Rad One-Step RT-ddPCR Advanced Kit for Probes 1864021) was performed on the extracted RNA to determine the copy number per cell of 8 selected genes. 3) Approximately 5000 Jurkat cells were processed using the GemCode Single Cell 3′ platform, and single stranded cDNA was collected after RT in GEMs following the protocols listed in “Sequencing library construction using the GemCode platform”. cDNA copies of the 8 genes were determined using ddPCR (Bio-Rad ddPCR Supermix for Probes (no dUTP) P/N 1863024). The actual Jurkat cell count was found by sequencing a subset of the GEM-RT reactions on a MiSeq. The conversion efficiency is the ratio between cDNA copies per cell (step 3) and RNA copies per cell from bulk RT-ddPCR (step 2), assuming a 50% efficiency in RT-ddPCR.

The probe sequences for the ddPCR assay are as follows.

SERAC1_f:
(SEQ ID NO: 2)
CACGAGCCGCCAGC;
SERAC1_r:
(SEQ ID NO: 3)
TCTGCAACAGATGACGCAATAAG;
SERAC1_p:
(SEQ ID NOS 4 and 5, respectively)
/56-FAM/CGCCTGCCG/ZEN/GCAGAATGTC/3IABkFQ/.
AP1S3_f:
(SEQ ID NO: 6)
GAAGCAGCCATGGTCTAAGC;
AP1S3_r:
(SEQ ID NO: 7)
CCTTGTCGACTGAAGAGCAATATG;
AP1S3_p:
(SEQ ID NOS 8 and 9, respectively)
/56-FAM/CGGCCCAGC/ZEN/CACGATGATACAT/3IABkFQ/.
ORAOV1_f:
(SEQ ID NO: 10)
CCGGAAGTGGGTCTCGT;
ORAOV1_r:
(SEQ ID NO: 11)
TTCTTCATAGCCTTCCCGATACC;
ORAOV1_p:
(SEQ ID NOS 12 and 13, respectively)
/56-FAM/TCGTGATGG/ZEN/CGGATGAGAGGTTTCA/3IABkFQ/.
DOLPP1_f:
(SEQ ID NO: 14)
ATGGCAGCGGACGGA;
DOLPP1_r:
(SEQ ID NO: 15)
GGCTCAGGTAGGCAAGGA;
DOLPP1_p:
(SEQ ID NOS 16 and 17, respectively)
/56-FAM/CCACGTCGA/ZEN/ATATCCTGCAGGTGATCT/3IABkFQ/.
KPNA6_f:
(SEQ ID NO: 18)
TGAAAGCTGCCGCTGAAG;
KPNA6_r:
(SEQ ID NO: 19)
CCCTGGGCTCGCCAT;
KPNA6_p:
(SEQ ID NOS 20 and 21, respectively)
/56-FAM/CGGACCCGC/ZEN/GATGGAGACC/3IABkFQ/.
ITSN2_f:
(SEQ ID NO: 22)
GTGACAGGCTACGCAACAG;
ITSN2_r:
(SEQ ID NO: 23)
TCCTGAGTTTTCCTTGCTAGCT;
ITSN2_p:
(SEQ ID NOS 24 and 25, respectively)
/56-FAM/AGGGCGCCA/ZEN/GATGGCTGA/3IABkFQ/.
LCMT1_f:
(SEQ ID NO: 26)
GTCGACCCCGCTTCCA;
LCMT1_r:
(SEQ ID NO: 27)
GGTCATGCCAGTAGCCAATG;
LCMT1_p:
(SEQ ID NOS 28 and 29, respectively)
/56-FAM/ATGCTTCCC/ZEN/TGTGCAAGAGGTTTGC/3IABkFQ/.
AP2M1_f:
(SEQ ID NO: 30)
GCAGCGGGCAGACG;
AP2M1_r:
(SEQ ID NO: 31)
ATGGCGGCAGATCAGTCT;
AP2M1_p:
(SEQ ID NO: 32 and 33, respectively)
/56-FAM/CATCGCTCT/ZEN/GAGAACAGACCTGGTG/3IABkFQ/.

Cell Capture Efficiency Calculation

The efficiency was calculated by taking the ratio of the number of cells detected by sequencing vs. the number of cells loaded into the chip. The latter was determined from (volume added*input concentration of cells), and takes into account losses in the chip. These losses include: 1) cells left behind in sample well, 2) cells in GEMs left behind in the outlet well, 3) cells in GEMs with Nbead=0 and Nbead>1. The losses do not include cells left behind in pipette tips during mixing and transfer steps before pipetting into the sample well. The theoretical efficiency (based on the Cell Loading Correction Factor of 1.92) is 52%. There was approximately 15-20% error in cell counts, which could account for at least some of the variability in the calculated efficiencies.

Chimerism Assay

PowerPlex 16 System (Promega) was used in conjunction with an Applied Biosystems (Life Technologies) 3130xI Genetic Analyzer. Donor BMMCs were used as the reference baseline.

Alignment, Barcode Assignment and UMI Counting

The Cell Ranger Single Cell Software Suite was used to perform sample demultiplexing, barcode processing, and single cell 3′ gene counting (http://software.10xgenomics.com/single-cell/overview/welcome). First, sample demultiplexing was performed based on the 8 bp sample index read to generate FASTQs for the Read1 and Read2 paired-end reads as well as the 14 bp GemCode barcode. 10 bp UMI tags were extracted from Read2. Then, Read1, which contains the cDNA insert, was aligned to an appropriate reference genome using STAR. For mouse cells, mm10 was used. For human cells, hg19 was used. For samples with mouse and human cell mixtures, the union of hg19 and mm10 were used. For ERCC samples, ERCC reference (https://tools.thermofisher.com/content/sfs/manuals/cms_095047.txt) was used.

Next, GemCode barcodes and UMIs were filtered. All of the known listed of barcodes that are 1-Hamming-distance away from an observed barcode were considered. Then the posterior probability that the observed barcode was produced by a sequencing error was computed, given the base qualities of the observed barcode and the prior probability of observing the candidate barcode (taken from the overall barcode count distribution). If the posterior probability for any candidate barcode was at least 0.975, then the barcode was corrected to the candidate barcode with the highest posterior probability. If all candidate sequences are equally probable, then the one appearing first by lexical order was picked.

UMIs with sequencing quality score>10 were considered valid if they were not homopolymers. A UMI that is 1-Hamming-distance away from another UMI (with more reads) for the same cell barcode and gene was corrected to the UMI with more reads. This approach is nearly identical to that in Jaitin et al., and is similar to that in Klein et al. (although Klein et al. also used UMIs to resolve multi-mapped reads, which was not implemented here).

Lastly, PCR duplicates were marked if two sets of read pairs shared a barcode sequence, a UMI tag, and a gene ID (Ensembl GTFs GRCh37.82, ftp://ftp.ensembl.org/pub/grch37/release-84/gtf/homo_sapiens/Homo_sapiens.GRCh37.82.gtf.gz, and GRCm38.84, ftp://ftp.ensembl.org/pub/release-84/gtf/mus_musculus/Mus_musculus.GRCm38.84.gtf.gz, were used). Confidently mapped (MAPQ=255), non-PCR duplicates with valid barcodes and UMIs were used to generate gene-barcode matrix.

Cell barcodes were determined based on distribution of UMI counts. All top barcodes within the same order of magnitude (greater than 10% of the top nth barcode where n is 1% of the expected recovered cell count) were considered cell barcodes. Number of reads that provide meaningful information is calculated as the product of 4 metrics: 1) valid barcodes; 2) valid UMI; 3) associated with a cell barcode; and 4) confidently mapped to exons.

In the mouse and human mixing experiments, multiplet rate was defined as twice the rate of cell barcodes with significant UMI counts from both mouse and human, where top 1% of UMI counts was considered significant. The extent of barcode crosstalk was assessed by the fraction of mouse reads in human barcodes, or vice versa.

Samples processed from multiple channels can be combined by concatenating gene-cell-barcode matrices. This functionality is provided in the Cell Ranger R Kit. Sequencing data from multiple sequencing runs of a library can be combined by counting non-duplicated reads. This functionality is provided in the Cell Ranger pipeline. In addition, sequencing data can be subsampled to obtain a given number of UMI counts per cell. This functionality is also provided in the Cell Ranger R Kit, and can be useful when combining data from multiple samples for comparison.

PCA Analysis of Mixing of Jurkat and 293T Cells

Gene-cell-barcode matrix from each of the 4 samples was concatenated. Only genes with at least 1 UMI count detected in at least 1 cell were used. UMI normalization was performed by first dividing UMI counts by the total UMI counts in each cell, followed by multiplication with the median of the total UMI counts across cells. Then the natural log of the UMI counts was taken. Finally, each gene was normalized such that the mean signal for each gene was 0, and standard deviation was 1. PCA was run on the normalized gene-barcode matrix. The normalized UMI counts of each gene were used to show expression of a marker in a tSNE plot.

SNV Analysis of Jurkat and 293T Cells

SNVs were called by running Freebayes 1.0.2 on the genome BAM produced by Cell Ranger. High quality SNVs (SNV calling Qual>=100 with at least 10 UMI counts from at least 2 cells; indels ignored) that were only observed in Jurkat or 293T cells (but not both) were selected. Cells were labeled as Jurkat or 293T based on Jurkat- and 293T-specific SNV counts, where the fraction of counts from the other species is <0.2. Cells with fraction of SNV from either species between 0.2 and 0.8 were considered multiplets. The inferred multiplet rate is 2*observed multiplet rate (to account for Jurkat:Jurkat and 293T:293T multiplets).

PCA and t-SNE Analysis of PBMCs

Genes with at least 1 UMI count detected in at least 1 cell were used. Top 1,000 most variable genes were identified based on their mean and dispersion (variance/mean), which is similar to the approach used by Macoscko et al. Genes were placed into 20 bins based on their mean expression. Normalized dispersion was calculated as the absolute difference between dispersion and median dispersion of the expression mean, normalized by median absolute deviation within each bin.

PCA was run on the normalized gene-barcode matrix of the top 1,000 most variable genes to reduce the number of feature (gene) dimensions. UMI normalization was performed by first dividing UMI counts by the total UMI counts in each cell, followed by multiplication with the median of the total UMI counts across cells. Then the natural log of the UMI counts was taken. Finally, each gene was normalized such that the mean signal for each gene was 0, and standard deviation was 1. PCA was run on the normalized gene-barcode matrix. After running PCA, Barnes-hut approximation to t-distributed Stochastic Neighbor Embedding (t-SNE) was performed on the first 50 PCs to visualize cells in a 2-D space. K-means clustering was run to group cells for the clustering analysis. k=10 was selected based on the sum of squared error scree plot.

Identification of Cluster-Specific Genes and Marker-Based Classification

To identify genes that are enriched in a specific cluster, mean expression of each gene was calculated across all cells in the cluster. Then each gene from the cluster was compared to the median expression of the same gene from cells in all other clusters. Genes were ranked based on their expression difference, and top 10 enriched genes from each cluster were selected. For hierarchical clustering, pair-wise correlation between each cluster was calculated, and centered expression of each gene was used for visualization by heatmap.

Classification of PBMCs was inferred from the annotation of cluster-specific genes. In the case of cluster 10, marker expression of multiple cell types (e.g. B, dendritic, and T) was detected. Since the relative cluster size of B, dendritic and T was 5.7%, 6.6% and 81% respectively, it was expected that cluster 10 (which is only 0.5%) contained multiplets consisting mostly from B:dendritic (0.36%) and B:dendritic:T (0.3%).

Selection of Purified Sub-Populations of PBMCs

Each population of purified PBMCs was downsampled to ˜16 k reads per cell. PCA, tSNE and k-means clustering were performed for each downsampled matrix, following the same steps outlined in PCA and t-SNE analysis of PBMCs. Only one cluster was detected in most samples, consistent with the FACS analyses. For samples with more than one cluster, only clusters that displayed the expected marker gene expression were selected for downstream analysis. For CD14+ Monocytes, 2 clusters were observed and identified as CD14+ Monocytes and Dendritic cells based on expression of marker genes FTL and CLEC9A, respectively.

Cell Classification Analysis Using Purified PBMCs

Each population of purified PBMCs was downsampled to ˜16 k confidently mapped reads per cell. Then, an average (mean) gene expression profile across all cells was calculated. Next, gene expression from every cell of the complex population was compared to the gene expression profiles of purified populations of PBMCs by spearman correlation. The cell was assigned the ID of the purified population if it had the highest correlation with that population. Note that the difference between the highest and 2nd highest correlation was small for some cells (for example, the difference between cytotoxic T and NK cells), suggesting that the cell assignment was not as confident for these cells. A few of the purified PBMC populations overlapped with each other. For example, CD4+ T Helper 2 cells include all CD4+ cells. This means that cells from this sample will overlap with cells from samples that contain CD4+ cells, including CD4+/CD25+ T Reg, CD4+/CD45RO+ T Memory, CD4+/CD45RA+/CD25− naïve T. Thus, when a cell was assigned the ID of CD4+ T Helper 2 cell based on the correlation score, the next highest correlation was checked to see if it was one of the CD4+ samples. If it was, the cell's ID was updated to the cell type with the next highest correlation. The same procedure was performed for CD8+ cytotoxic T and CD8+/CD45RA+ naïve cytotoxic T (which is a subset of CD8+ cytotoxic T).

The R code used to analyze 68 k PBMCs and purified PBMCs can be found here:

http://software.10xgenomics.com/single-cell/downloads/latest.

Cell Clustering and Classification with Seurat

The gene-cell-barcode matrix of 68 k PBMCs was log-transformed as an input to Seurat. The top 469 most variable genes selected by Seurat were used to compute the PCs. The first 22 PCs were significant (p<0.01) based on the built-in jackstraw analysis, and used for tSNE visualization. Cell classification was taken from Cell classification analysis using purified PBMCs.

Cell Classification Comparison Between Purified and Frozen PBMCs

Since the sub-populations within T and NK cells are similar, thus challenging to form distinct clusters, all the cells labeled as T or NK cells were pooled together.

SNV-Based Genotype Assignment

SNVs were called by running Freebayes 1.0.2 on the genome BAM produced by Cell Ranger. SNVs with support from at least 2 cell barcodes, with a minimal SNV Qual score >=30, minimal SNV base Qual>=1 were included. Reference (R) and alternate (A) allele counts were computed at each SNV, producing a matrix of cell-reference UMI counts and cell-alternate-allele UMI counts. These matrices were modeled as a mixture of two genomes where the likelihood of any of the three genotypes (R/R, R/A, or A/A) at a site was taken to be binomially distributed with a fixed error rate of 0.1%. For each sample, two models were inferred in parallel, one where only one genome is present (K=1) and another where two genomes are present (K=2). Inference of the model parameters (cell-to-genome assignments and the K sets of genotypes) was performed by using a Gibbs sampler to approximate their posterior distributions. In order to ameliorate the label-switching problem in Monte Carlo inference of mixture models, relabeling of the sampled cell-to-genome assignments was performed as per Stephens et al.

In in silico cell mixing experiments, when the K=2 model failed to adequately separate the two genomes, it reported a distribution of posterior probabilities near 0.5 for the cell-genome calls, indicating a lack of confidence in those calls. A requirement that 90% of the cells have a posterior probability greater than 75% in order to select the K=2 model over the K=1 model was applied. Selecting K=1 indicates that the mixture fraction is below the level of detection of the method, which in in silico mixing experiments was determined to be 4% of 6,000 cells.

Genotype Comparison to the Pure Sample

To ascertain the assignment of genotypes to individuals, shared SNVs between the genotype group and the pure sample were considered. Then the average genotype of all the cells was compared to that of the pure sample. In order to obtain some baseline for the % genotype overlap among different individuals, pairwise comparison of genotypes called from the same individuals (11 pairwise comparisons) or from different individuals (15 pairwise comparisons) was performed. The percent genotype overlap between the same individuals averages ˜98%±0.3%, whereas the percent genotype overlap between the different individuals averages ˜73%±2%.

PCA and t-SNE Analysis of BM MCs

Data from 6 samples were used: 2 healthy controls, AML027 pre- and post-transplant, and AML035 pre- and post-transplant. Each sample was downsampled to ˜10 k confidently mapped reads per cell. Then the gene-cell barcode matrix from each sample was concatenated. PCA, tSNE and k-means clustering were performed on the pooled matrix, following the same steps outlined in PCA and t-SNE analysis of PBMCs. For k-means clustering, k=10 was used based on the bend in the sum of squared error scree plot.

Cluster-specific genes were identified following the steps outlined in Identification of cluster-specific genes and marker-based classification. Classification was assigned based on cluster-specific genes, and based on expression of some well-known markers of immune cell types. “Blasts and Immature Ery 1” refers to cluster 4, which expresses CD34, a marker of hematopoietic progenitors, and Gata2, a marker for early erythroids. “Immature Ery 2” refers to clusters 5 and 8, which show expression of Gata1, a transcription factor essential for erythropoiesis, but not CD71, which are often found in more committed erythroid cells. “Immature Ery 3” refers to cluster 1, which show expression of CD71. “Mature Ery” refers to cluster 2. HBA1, a marker of mature erythroid cells, is preferentially detected in cluster 2. Cluster 3 was assigned as “Immature Granulocytes” because of the expression of early granulocyte markers such as AZU1 and IL8, and the lack of expression of CD16. Cluster 7 was assigned as “Monocytes” because of the expression of CD14 and FCN1, for example. “B” refers clusters 6 and 9 because of markers such as CD19 and CD79A. “T” refers to cluster 10, because of markers such as CD3D and CD8A.

While some embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1-99. (canceled)

100. A method of distinguishing a minor cell population from a major cell population in a heterogeneous cell sample, comprising:
(a) partitioning a plurality of cells of a heterogeneous cell sample into a plurality of droplets, wherein upon partitioning, a given droplet of said plurality of droplets comprises a given cell of said plurality of cells and a given bead of a plurality of beads comprising a plurality of oligonucleotide barcodes, wherein said given cell comprises a set of RNA transcripts, wherein a plurality of oligonucleotide barcodes of said given bead comprise (i) a barcode sequence identical to all other of said plurality of oligonucleotide barcodes of said given bead, and (ii) a unique molecular identifier (UMI) sequence not identical to a UMI of other of said plurality of oligonucleotide barcodes of said given bead;
(b) in said given droplet, applying a stimulus to said given droplet to degrade said given bead, thereby releasing said oligonucleotide barcodes from said given bead into said given droplet;
(c) in said given droplet, subjecting said set of RNA transcripts to nucleic acid amplification under conditions sufficient to generate a set of polynucleotides, wherein a given polynucleotide of said set of polynucleotides comprises (i) a segment having a sequence of an RNA transcript of said set of RNA transcripts or a complement thereof and (ii) a segment having a sequence of a oligonucleotide barcode of said plurality of oligonucleotide barcodes or a complement thereof;
(d) generating a library of polynucleotides from said set of polynucleotides;
(e) subjecting said library of polynucleotides to sequencing to yield sequencing reads, wherein barcode sequences of said plurality of oligonucleotide barcodes associate sequencing reads with individual cells of said plurality of cells of said heterogeneous cell sample; and
(f) processing said sequencing reads associated with individual cells of said plurality of cells of said heterogeneous cell sample to generate (i) a first set of genetic aberrations corresponding to said minor cell population and (ii) a second set of genetic aberrations corresponding to said major cell population, which first and second set of genetic aberrations differentiate a cell of said minor cell population from a cell of said major cell population, wherein said first set of genetic aberrations and said second set of genetic aberrations comprise single nucleotide variants (SNVs).

101. The method of claim 100, wherein said SNVs comprise an SNV located in an untranslated region (UTR) of the RNA transcript.

102. The method of claim 101, wherein said UTR of the RNA transcript is a 3′ UTR of the transcript.

103. The method of claim 100, wherein about 50% of said plurality of cells are associated with said sequencing reads.

104. The method of claim 100, further comprising, subsequent to (a), releasing said first set of polynucleotides from said given cell into said given droplet.

105. The method of claim 100, wherein said given bead of said given droplet is a gel bead.

106. The method of claim 100, wherein said given bead of said given droplet comprises at least 1,000,000 oligonucleotide barcodes.

107. The method of claim 100, wherein each of said first and second set of genetic aberrations comprises at least 30 SNVs.

108. The method of claim 100, wherein said first set of genetic aberrations and said second set of genetic aberrations do not intersect (do not share members).

109. The method of claim 100, wherein said major cell population comprises at least two cell types.

110. The method of claim 100, wherein said minor cell population represents less than 50% of said heterogeneous cell sample.

111. The method of claim 110, wherein said minor cell population represents greater than or equal to about 1% of said heterogeneous cell sample.

112. The method of claim 100, further comprising determining a percentage of said heterogeneous cell sample represented by said major cell population.

113. The method of claim 112, wherein said major cell population represents greater than about 50% of said heterogeneous cell sample.

114. The method of claim 100, further comprising determining a percentage of said heterogeneous cell sample represented by said minor cell population.

115. The method of claim 114, wherein said percentage of said heterogeneous cell sample represented by said minor cell population is determined at a sensitivity of at least about 95%.

116. The method of claim 100, wherein nucleic acid amplification reagents are co-partitioned in said given droplet.

117. The method of claim 116, wherein said nucleic acid amplification reagents comprise a template switching oligonucleotide.

118. The method of claim 100, wherein said heterogeneous cell sample comprises cells obtained from a biological sample.

119. A method of profiling untranslated regions of a transcriptome, comprising:
(a) partitioning a plurality of cells into a plurality of droplets, wherein upon partitioning, a given droplet of said plurality of droplets comprises a given cell of said plurality of cells and a given bead of a plurality of beads comprising a plurality of oligonucleotide barcodes, wherein said given cell comprises a set of RNA transcripts, wherein a plurality of oligonucleotide barcodes of said given bead comprise (i) a barcode sequence identical to all other of said plurality of oligonucleotide barcodes of said given bead, and (ii) a unique molecular identifier (UMI) sequence not identical to a UMI of other of said plurality of oligonucleotide barcodes of said given bead;
(b) in said given droplet, applying a stimulus to said given droplet to degrade said given bead, thereby releasing said oligonucleotide barcodes from said given bead into said given droplet;
(c) in said given droplet, subjecting said set of RNA transcripts to reverse transcription under conditions sufficient to generate a set of polynucleotides, wherein a given polynucleotide of said set of polynucleotides comprises (i) a segment having a sequence complementary to an untranslated region (UTR) of an RNA transcript of said set of RNA transcripts and (ii) a segment having a sequence of a oligonucleotide barcode of said plurality of oligonucleotide barcodes or a complement thereof;
(d) generating a library of polynucleotides from said second set of polynucleotides; and
(e) subjecting said library of polynucleotides to sequencing to yield sequencing reads, wherein barcode sequences of said plurality of oligonucleotide barcodes associate sequencing reads with individual cells of said plurality of cells, wherein a sequencing read of said sequencing reads is used to determine a sequence of said UTR of said RNA transcript of said set of RNA transcripts.
